(19) United States
(12) Patent Application Publication
(10) Pub. No.: US 2025/0051742 A1
(43) Pub. Date: Feb. 13, 2025

(54) ENGINEERED CAS12B EFFECTOR PROTEINS AND METHODS OF USE THEREOF

(71) Applicants: Beijing Institute for Stem Cell and Regenerative Medicine, Beijing (CN); Institute of Zoology, Chinese Academy of Sciences, Beijing (CN)

(72) Inventors: Wei LI, Beijing (CN); Qi ZHOU, Beijing (CN); Yangcan CHEN, Beijing (CN)

(21) Appl. No.: 18/717,976

(22) PCT Filed: Dec. 9, 2022
(86) PCT No.: PCT/CN2022/137920

§ 371 Date: Jun. 7, 2024

(30) Foreign Application Priority Data
Dec. 9, 2021 (WO) PCT/CN2021/136761

(51) Int. Cl. C12N 9/22; A61K 48/00; C12N 15/11; C12Q 1/6823

(52) U.S. Cl. CPC C12N 9/22; A61K 48/005; C12N 15/111; C12Q 1/6823; C12N 2310/20

(57) ABSTRACT

Provided are engineered Cas12b nucleases or derivatives thereof comprising one or more types of mutations with improved activity (e.g, gene editing activity) or abolished nuclease activity. Also provided are engineered Cas12b effector proteins, engineered gRNAs (e.g, sgRNAs or tracrRNAs), engineered CRISPR-Cas12b systems, and methods of use thereof.

### CROSS REFERENCE TO RELATED APPLICATIONS

This application claims priority benefit of International Patent Application No. PCT/CN2021/136761 filed Dec. 9, 2021, the content of which is incorporated herein by reference in its entirety.

### REFERENCE TO AN ELECTRONIC SEQUENCE LISTING

The content of the electronic sequence listing (253112000541SEQLIST.xml; Size: 111,583 bytes; and Date of Creation: Nov. 22, 2022) is herein incorporated by reference in its entirety.

### FIELD

The present application relates generally to the field of biotechnology. More specifically, the present application relates to engineered Cas12b effector proteins and engineered gRNA scaffolds with improved activity (e.g., gene editing activity) or abolished nuclease activity, and methods of use thereof.

### BACKGROUND

Genome editing is an important and useful technology in genomic research and various applications. Various systems may be used for genome editing, including the clustered regularly interspersed short palindromic repeats (CRISPR)-Cas system, the transcription activator-like effector nuclease (TALEN) system, and the zinc finger nuclease (ZFN) system.

The CRISPR-Cas system is an efficient and cost-effective genome-editing technology that is widely applicable in a range of eukaryotic organisms from yeast and plants to zebrafish and human (reviewed by Van der Oost 2013, Science 339: 768-770, and Charpentier and Doudna, 2013, Nature 495: 50-51). The CRISPR-Cas system provides adaptive immunity in archaea and bacteria by employing a combination of Cas effector proteins and CRISPR RNAs (crRNAs). To date, two classes (class 1 and 2) including six types (type I-VI) of CRISPR-Cas systems have been characterized according to prominent functional and evolutionary modularity of the systems. Among class 2 CRISPR-Cas systems, type II Cas9 systems and type V-A/B/E/J Cas12a/Cas12b/Cas12e/Cas12f/Cas12j systems have been harnessed for genome editing, and hold tremendous promise for biomedical research.

### BRIEF SUMMARY

Current CRISPR-Cas systems have various limitations, including limited gene-editing efficiency. The present application provides improved methods and systems for effective genome editing across a variety of genomic loci. Particularly, provided herein are engineered Cas12b nucleases having improved enzymatic activity, engineered Cas12b effector proteins, engineered gRNAs (e.g., sgRNAs or tracrRNAs) comprising engineered scaffolds, and methods of using the engineered Cas12b effector proteins and/or engineered gRNAs, such as in gene editing. In one aspect, the present application provides engineered Cas12b nuclease, comprising one, two, or three types of mutations with respect to a reference Cas12b nuclease, wherein the mutations comprise: (1) substitution of one or more amino acid residues in the reference Cas12b nuclease that interact with a protospacer adjacent motif (PAM) with a positively charged amino acid residue (e.g., R, H, K); and/or (2) substitution of one or more amino acid residues in the reference Cas12b nuclease that are involved in opening DNA double strands (dsDNA) with an amino acid residue having an aromatic ring (e.g., F, Y, W); and/or (3) substitution of one or more amino acid residues in the RuvC domain of the reference Cas12b nuclease that interact with a single-stranded DNA (ssDNA) substrate with a positively charged amino acid residue (e.g., R, H, K) or a hydrophobic amino acid residue (e.g., F, Y, W, M). In some embodiments, the reference Cas12b nuclease is a wild-type Cas12b nuclease. In some embodiments, the reference Cas12b nuclease is a Cas12b nuclease from Alicyclobacillus acidiphilus (AaCas12b). In some embodiments, the reference Cas12b nuclease comprises the amino acid sequence of SEQ ID NO: 1.

In some embodiments according to any one of the engineered Cas12b nucleases described above, the engineered Cas12b nuclease comprises substitution of one or more amino acid residues in the reference Cas12b nuclease that interact with PAM with a positively charged amino acid residue. In some embodiments, the one or more amino acid residues that interact with PAM are within 10 (e.g., 9, 8, 7, 6, 5, 4, 3, 2, 1, or less) angstroms from PAM in a three-dimensional structure. In some embodiments, the one or more amino acid residues that interact with PAM are in one or more of the following positions: 116, 123, 130, 132, 144, 145, 153, 173, 222, 395, 400, and 475. In some embodiments, the one or more amino acid residues that interact with PAM comprise one or more of the following amino acid residues: D116, K123, D130, D132, N144, K145, E153, D173, Q222, D395, N400, and E475. In some embodiments, the one or more amino acid residues that interact with PAM comprise one or more of the following amino acid residues: D116 and E475. In some embodiments, the positively charged amino acid residue is R or K. In some embodiments, the engineered Cas12b nuclease comprises one or more of the following substitutions: D116R and E475R. In some embodiments, the amino acid residue numbering is according to SEQ ID NO: 1. In some embodiments, the engineered Cas12b nuclease comprises the amino acid sequence of SEQ ID NO: 2 or 3.

In some embodiments according to any one of the engineered Cas12b nucleases described above, the engineered Cas12b nuclease comprises substitution of one or more amino acid residues in the reference Cas12b nuclease that are involved in opening the DNA double strands with an amino acid residue having an aromatic ring. In some embodiments, the one or more amino acid residues that are involved in opening the DNA double strands interact with the last base pair in PAM relative to the 3′end of a target strand. In some embodiments, the one or more amino acid residues that are involved in opening the DNA double strands are in one or more of the following positions: 118 and 119. In some embodiments, the amino acid residue having an aromatic ring is Y, F, or W. In some embodiments, the substitution of one or more amino acid residues in the reference Cas12b nuclease that are involved in opening the DNA double strands with the amino acid residue having an aromatic ring is Q119Y, Q119F, or Q119W. In some embodiments, the amino acid residue numbering is according to SEQ ID NO: 1. In some embodiments, the engineered Cas12b nuclease comprises the amino acid sequence of any of SEQ ID NOs: 4-6.

In some embodiments according to any one of the engineered Cas12b nucleases described above, the engineered Cas12b nuclease comprises substitution of one or more amino acid residues in the reference Cas12b nuclease that are in the RuvC domain and interact with a single-stranded DNA substrate with a positively charged amino acid residue or a hydrophobic amino acid residue. In some embodiments, the one or more amino acid residues that are in the RuvC domain and interact with the single-stranded DNA substrate are within 10 (e.g., 9, 8, 7, 6, 5, 4, 3, 2, 1, or less) angstroms from the single-stranded DNA substrate in a three-dimensional structure. In some embodiments, the one or more amino acid residues that are in the RuvC domain and interact with the single-stranded DNA substrate are in one or more of the following positions: 300, 301, 304, 329, 636, 639, 647, 682, 757, 758, 761, 764, 768, 852, 854, 856, 857, 858, 860, 862, 863, 865, 866, 867, 869, 938, 956, 957, 958, 994, 1093, and 1097. In some embodiments, the one or more amino acid residues that are in the RuvC domain and interact with the single-stranded DNA substrate comprise one or more of the following amino acid residues: D300, K301, E304, N329, E636, Q639, T647, Q682, I757, E758, E761, E764, K768, E852, Q854, N856, N857, D858, P860, S862, E863, N865, Q866, L867, Q869, E938, E956, G957, E958, I994, Q1093, and W1097. In some embodiments, the engineered Cas12b nuclease comprises substitution of one or more of the following amino acid residues with a positively charged amino acid residue: E636, Q639, T647, Q682, I757, E758, E761, K768, Q854, N857, D858, N865, Q866, I994, Q1093, and W1097. In some embodiments, the positively charged amino acid residue is R or K. In some embodiments, the engineered Cas12b nuclease comprises one or more of the following substitutions: E636R, Q639R, T647R, Q682R, I757R, E758R, E761R, Q854R, N857K, D858R, I994R, Q1093R, and W1097R. In some embodiments, the engineered Cas12b nuclease comprises substitution of one or more of the following amino acid residues with a hydrophobic amino acid residue: E758, E761, E863, N865, Q866, Q869, Q956, and Q1093. In some embodiments, the hydrophobic amino acid residue is W, Y, F, or M, such as W, Y or M. In some embodiments, the engineered Cas12b nuclease comprises one or more of the following substitutions: N865W, N865Y, Q866M, Q869M, Q1093W, and Q1093Y. In some embodiments, the amino acid residue numbering is according to SEQ ID NO: 1. In some embodiments, the engineered Cas12b nuclease comprises the amino acid sequence of any of SEQ ID NOs: 7-19.

In some embodiments according to any one of the engineered Cas12b nucleases described above, the engineered Cas12b nuclease comprises any one of the following substitutions or combinations thereof: (1) D116R; (2) E475R; (3) Q119F and E475R; (4) Q119F, E475R, and E758R; (5) Q119Y; (6) Q119F; (7) Q119W; (8) I757R; (9) E758R; (10) E761R; (11) K768R; (12) I757R and E758R; (13) I757R and E761R; (14) 1757R and K768R; (15) E758R and E761R; (16) E758R and K768R; (17) E761R and K768R; (18) I757R, E758R, and E761R; (19) I757R, E758R, and K768R; (20) I757R, E761R and K768R; (21) E758R, E761R, and K768R; (22) I757R, E758R, E761R, and K768R; (23) Q866M; (24) Q869M; (25) Q866M and Q869M; (26) E636R; (27) Q854R; (28) N857K; (29) N865W; (30) N865Y; (31) Q1093W; (32) Q1093Y; and (33) D858R; and wherein the amino acid residue numbering is according to SEQ ID NO: 1. In some embodiments, the engineered Cas12b nuclease comprises any one of the following substitutions or combinations thereof: (1) Q866M+Q869M; (2) Q119F+E475R; and (3) Q119F+E475R+E758R; and wherein the amino acid residue numbering is according to SEQ ID NO: 1. In some embodiments, the engineered Cas12b nuclease comprises an amino acid sequence of any one of SEQ ID NOs: 20-22.

In some embodiments according to any one of the engineered Cas12b nucleases described above, the engineered Cas12b nuclease comprises an amino acid sequence having at least about 85% (e.g., at least about any of 88%, 90%, 95%, 96%, 97%, 98%, 99%, or more) sequence identity to any one of SEQ ID NOs: 1-22. In some embodiments, the engineered Cas12b nuclease comprises (or consists of, or consists essentially of) the amino acid sequence of any one of SEQ ID NOs: 2-22.

In some embodiments according to any one of the engineered Cas12b nucleases described above, the engineered Cas12b nuclease further comprises one or more mutations in the reference Cas12b nuclease that increase flexibility of a flexible region comprising amino acid residues 855-859. In some embodiments, the one or more mutations that increase flexibility comprises N856G. In some embodiments, the amino acid position numbers are in reference to SEQ ID NO: 1.

One aspect of the present application provides an engineered Cas12b nuclease comprising any one or more of the following mutations: (1) D116R; (2) E475R; (3) Q119F+E475R; (4) Q119F+E475R+E758R; (5) Q119Y; (6) Q119F; (7) Q119W; (8) I757R; (9) E758R; (10) E761R; (11) K768R; (12) I757R+E758R; (13) I757R+E761R; (14) I757R+K768R; (15) E758R+E761R; (16) E758R+K768R; (17) E761R+K768R; (18) I757R+E758R+E761R; (19) I757R+E758R+K768R; (20) I757R+E761R+K768R; (21) E758R+E761R+K768R; (22) I757R+E758R+E761R+K768R; (23) Q866M; (24) Q869M; (25) Q866M+Q869M; (26) E636R; (27) Q854R; (28) N857K; (29) N865W; (30) N865Y; (31) Q1093W; (32) Q1093Y; and (33) D858R; and wherein the amino acid position number is in reference to SEQ ID NO: 1. In some embodiments, the engineered Cas12b nuclease comprises any one of the following substitutions or combinations thereof: (1) Q866M+Q869M; (2) Q119F+E475R; and (3) Q119F+E475R+E758R; and wherein the amino acid residue numbering is according to SEQ ID NO: 1. In some embodiments, the engineered Cas12b nuclease comprises substitutions of Q119F+E475R+E758R; and wherein the amino acid residue numbering is according to SEQ ID NO: 1.

One aspect of the present application provides an engineered Cas12b nuclease having at least about 85% (e.g., at least about any of 88%, 90%, 95%, 96%, 97%, 98%, 99%, or more) sequence identity to any one of SEQ ID NOs: 2-22, or comprising (or consisting of, or consisting essentially of) the amino acid sequence of any one of SEQ ID NOs: 2-22.

One aspect of the present application provides an engineered Cas12b effector protein comprising the engineered Cas12b nuclease according to any one of the engineered Cas12b nucleases described above or a variant or functional derivative thereof. In some embodiments, the engineered Cas12b nuclease or a functional derivative thereof is enzymatically active. In some embodiments, the engineered Cas12b effector protein is capable of inducing a double-strand break in a DNA molecule. In some embodiments, the engineered Cas12b effector protein is capable of inducing a single-strand break in a DNA molecule. In some embodiments, the engineered Cas12b effector protein comprises an enzymatically inactive mutant of the engineered Cas12b nuclease. In some embodiments, the enzyme-inactivating mutant of the engineered Cas12b nuclease comprises substitution of one or more amino acid residues selected from the group consisting of D570A, E848A, R785A, E848A, R911A, and D977A, and wherein the amino acid residue numbering is according to SEQ ID NO: 1. In some embodiments, the enzymatically inactive mutant of the engineered Cas12b nuclease comprises (or consists of, or consists essentially of) the amino acid sequence of any of SEQ ID NOs: 79-81, or a variant thereof having at least about 85% (e.g., at least about any of 88%, 90%, 95%, 96%, 97%, 98%, 99%, or more) sequence identity to any one of SEQ ID NOs: 79-81.

In some embodiments according to any one of the engineered Cas12b effector proteins described above, the engineered Cas12b effector protein further comprises a functional domain fused to the engineered Cas12b nuclease or functional derivative thereof. In some embodiments, the functional domain is selected from the group consisting of a translation initiator domain, a transcription repressor domain, a transactivation domain, an epigenetic modification domain, a nucleobase-editing domain, a reverse transcriptase domain, a reporter domain, and a nuclease domain. In some embodiments, the transcription repressor domain is a Kruppel-associated box (KRAB) domain, such as comprising the amino acid sequence of SEQ ID NO: 72.

In some embodiments according to any one of the engineered Cas12b effector proteins described above, the engineered Cas12b effector protein comprises a first polypeptide comprising an N-terminal portion of the engineered Cas nuclease or functional derivative thereof, and a second polypeptide comprising a C-terminal portion of the engineered Cas nuclease or functional derivative thereof, wherein the first polypeptide and the second polypeptide are capable of associating with each other in the presence of a guide RNA comprising a guide sequence to form a CRISPR complex that specifically binds to a target nucleic acid comprising a target sequence complementary to the guide sequence. In some embodiments, the engineered Cas12b effector protein comprises a first polypeptide and a second polypeptide, wherein the first polypeptide comprises the N-terminal amino acid residues 1 to X of the engineered Cas12b nuclease or functional derivative thereof, wherein the second polypeptide comprises the X+1 residue to the C-terminus of the engineered Cas12b nuclease or functional derivative thereof, wherein the first polypeptide and the second polypeptide are capable of associating with each other in the presence of a guide RNA containing a guide sequence to form a CRISPR complex that specifically binds to a target nucleic acid, wherein the target nucleic acid comprises a target sequence complementary to the guide sequence. In some embodiments, the first polypeptide and the second polypeptide each comprises a dimerization domain. In some embodiments, the first dimerization domain and the second dimerization domain associate with each other in the presence of an inducer. In some embodiments, the first polypeptide and the second polypeptide do not comprise dimerization domains.

Another aspect of the present application provides a single guide RNA (sgRNA) comprising the sequence of any one of SEQ ID NOs: 25-53.

Another aspect of the present application provides an engineered CRISPR-Cas12b system comprising: (a) the engineered Cas12b nuclease according to any one of the engineered Cas12b nucleases described above or the engineered Cas12b effector protein according to any one of the engineered Cas12b effector proteins described above, or a nucleic acid encoding thereof, and (b) a guide RNA comprising a guide sequence complementary to a target sequence of a target nucleic acid, or a nucleic acid encoding the guide RNA, wherein the engineered Cas12b nuclease or the engineered Cas12b effector protein and the guide RNA are capable of forming a CRISPR complex that specifically binds to the target nucleic acid comprising the target sequence and inducing a modification of the target nucleic acid. In some embodiments, the guide RNA comprises a crRNA and a tracrRNA. In some embodiments, the engineered CRISPR-Cas12b system comprises a precursor guide RNA array encoding a plurality of crRNAs. In some embodiments, the guide RNA is a single guide RNA (sgRNA). In some embodiments, the sgRNA comprises the sequence of any one of SEQ ID NOs: 23-53. In some embodiments, the engineered CRISPR-Cas12b system comprises comprising one or more vectors encoding the engineered Cas12b nuclease or the engineered Cas12b effector protein. In some embodiments, the one or more vector is an adeno-associated viral (AAV) vector. In some embodiments, the AAV vector further encodes the guide RNA.

Another aspect of the present application provides an engineered CRISPR-Cas12b system comprising: (a) an engineered Cas12b nuclease according to any one of the engineered Cas12b nucleases described above or an engineered Cas12b effector protein according to any one of the engineered Cas12b effector proteins described above, a Cas12b nuclease or an effector protein thereof comprising the amino acid sequence of any of SEQ ID NOs: 1-22 and 79-81, or a nucleic acid encoding thereof, and (b) a gRNA comprising a guide sequence complementary to a target sequence of a target nucleic acid, or a nucleic acid encoding the gRNA, wherein the gRNA comprises an engineered scaffold comprising the sequence of any of SEQ ID NOs: 25-53; wherein the Cas12b nuclease (e.g., engineered) or effector protein thereof and the gRNA are capable of forming a CRISPR complex that specifically binds to the target nucleic acid and inducing a modification of the target nucleic acid. In some embodiments, the gRNA comprises a crRNA and a tracrRNA, and wherein the tracrRNA comprises the engineered scaffold or portion thereof. In some embodiments, the engineered CRISPR-Cas12b system comprises a precursor gRNA array encoding a plurality of crRNAs. In some embodiments, the gRNA is an sgRNA. In some embodiments, the engineered CRISPR-Cas12b system comprises one or more vectors encoding the engineered Cas12b nuclease or effector protein thereof, or the Cas12b nuclease or effector protein thereof. In some embodiments, the one or more vectors are AAV vectors. In some embodiments, the one or more vectors further encode the gRNA.

One aspect of the present application provides a method of detecting target nucleic acid in a sample, including: (a) contacting the sample with the engineered CRISPR-Cas12b system according to any one of the engineered CRISPR-Cas12b systems described above and a labeled detector nucleic acid, wherein the gRNA comprises a guide sequence complementary to a target sequence of the target nucleic acid, and wherein the labeled detector nucleic acid is single-stranded and does not hybridize with the guide sequence of the guide RNA; and (b) measuring a detectable signal produced by cleavage of the labeled detector nucleic acid by the engineered Cas12b nuclease or effector protein thereof, thereby detecting the target nucleic acid.

One aspect of the present application provides a method of modifying a target nucleic acid comprising a target sequence, comprising contacting the target nucleic acid with the engineered CRISPR-Cas12b system according to any one of the engineered CRISPR-Cas12b systems described above. In some embodiments, the method is carried out in vitro. In some embodiments, the target nucleic acid is present in a cell. In some embodiments, the cell is a bacterial cell, a yeast cell, a plant cell, or an animal cell (e.g., a mammalian cell). In some embodiments, the method is carried out ex vivo. In some embodiments, the method is carried out in vivo. In some embodiments, the target nucleic acid is cleaved. In some embodiments, the target sequence in the target nucleic acid is altered by the engineered CRISPR-Cas12b system. In some embodiments, the expression of the target nucleic acid is altered by the engineered CRISPR-Cas12b system. In some embodiments, the target nucleic acid is a genomic DNA. In some embodiments, the target sequence is associated with a disease or condition. In some embodiments, the engineered CRISPR-Cas12b system comprises a precursor guide RNA array encoding a plurality of crRNA, wherein each crRNA comprises a different guide sequence.

Another aspect of the present application provides a method of treating a disease or a condition associated with a target nucleic acid in cells of an individual, comprising modifying the target nucleic acid in the cells of the individual using the engineered CRISPR-Cas12b system according to any one of the engineered CRISPR-Cas12b systems described above, thereby treating the disease or the condition. In some embodiments, the disease or condition is selected from the group consisting of cancer, cardiovascular diseases, hereditary diseases, autoimmune diseases, metabolic diseases, neurodegenerative diseases, ocular diseases, bacterial infections and viral infections.

Also provided are engineered cells comprising a modified target nucleic acid, wherein the target nucleic acid has been modified using the method according to any one of the methods of modifying a target nucleic acid described above. Also provided are engineered non-human animals comprising one or more engineered cells thereof.

Also provided are compositions, kits and articles of manufacture for use in any one the methods described above.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions, or components of a composition are specifically embraced by the present disclosure and are disclosed herein just as if each and every combination was individually and explicitly disclosed.

### BRIEF DESCRIPTION OF DRAWINGS

FIG. 1 shows the gene editing efficiencies (% indels) of exemplary AaCas12b variants, in which amino acid residues in wild-type AaCas12b that interact with PAM are substituted with R. The AaCas12b variants with D116R or E475R substitution showed improved editing efficiency as compared to the wild-type (WT) AaCas12b.

FIG. 2 shows the gene editing efficiencies of exemplary AaCas12b variants, in which amino acid residues in wild-type AaCas12b that are involved in opening DNA double strands are substituted with aromatic amino acid residues. The AaCas12b variants with Q119Y, Q119F, or Q119W substitution showed improved gene editing efficiency as compared to the WT AaCas12b.

FIG. 3 shows the gene editing efficiencies of exemplary AaCas12b variants, in which amino acid residues in wild-type AaCas12b that are in the RuvC domain and interact with single-stranded DNA substrate are substituted with R.

FIGS. 4A-4B show the gene editing efficiencies of exemplary AaCas12b variants, in which amino acid residues in wild-type AaCas12b that are in the RuvC domain and interact with a single-stranded DNA are substituted with lysine (K) or arginine (R) residues. FIG. 4A shows the editing efficiency at the genomic site CCR5-3 while FIG. 4B shows the editing efficiency at the genomic site RNF2-5. The AaCas12b variants with E636R, I757R, E758R, E761R, Q854R, D858R, E758K, I994R, N857K, or D858K substitution showed most improved gene editing efficiency as compared to the WT AaCas12b.

FIG. 5 shows the gene editing efficiencies of exemplary AaCas12b variants, in which amino acid residues in wild-type AaCas12b that are in the RuvC domain and interact with a single-stranded DNA substrate are substituted with hydrophobic amino acid residues W, Y, F, or M. The AaCas12b variants with N865W, N865Y, Q866M, Q869M, Q1093W, or Q1093Y substitution showed most improved gene editing efficiency as compared to the WT AaCas12b.

FIG. 6 shows the gene editing efficiencies of exemplary AaCas12b variants with combined mutations as compared to WT AaCas12b.

FIG. 7 shows the AaCas12b variant Q119F+E475R+E758R had significantly improved gene editing efficiency as compared to the WT AaCas12b and corresponding single mutants.

FIG. 8 shows alignments of amino acid sequences of Cas12b proteins, including Alicyclobacillus acidiphilus Cas12b (AaCas12b) (SEQ ID NO: 1), Alicyclobacillus kakegawensis Cas12b (AkCas12b) (SEQ ID NO: 54), Alicyclobacillus macrosporangiidus Cas12b (AmCas12b) (SEQ ID NO: 55), Bacillus sp. V3-13 Cas12b (Bs3Cas12b) (SEQ ID NO: 56), Bacillus Cas12b (BsCas12b) (SEQ ID NO: 57), Laceyella sediminis Cas12b (LsCas12b) (SEQ ID NO: 58), Bacillus hisashii Cas12b (BhCas12b) (SEQ ID NO: 59), and Spirochaetes bacterium Cas12b (SbCas12b) (SEQ ID NO: 60). Substitutions described herein based on AaCas12b can be made in any one of the Cas12b orthologues described herein at corresponding amino acid positions.

FIG. 9 shows that the sgRNAs with engineered scaffold greatly improved gene editing efficiency of the AaCas12b variant Q119F+E475R+E758R. sgRNA with AaCas12b Aa-sg scaffold or AacCas12b sgRNA scaffold (V0) served as control.

FIG. 10A is a schematic drawing of an exemplary construct encoding AaCas12b variant Q119F+E475R+E758R+D570A under the control of a CMV promoter, together with an sgRNA under the control of a U6 promoter. FIG. 10B shows T7EI assay results as a measure of the nuclease activity of AaCas12b (Q119F+E475R+E758R) and AaCas12b (Q119F+E475R+E758R+D570A). sgRNA1 and sgRNA2 specifically recognize target sites in HBG1/2. Control sgRNA not targeting any sequence of HBG1/2 served as negative control.

FIG. 11A is a schematic drawing of an exemplary construct encoding AaCas12b variant Q119F+E475R+E758R+D570A+E848A or Q119F+E475R+E758R+D570A+D977A under the control of a CMV promoter, together with an sgRNA under the control of a U6 promoter. FIG. 11B shows T7EI assay results as a measure of the nuclease activity of AaCas12b (Q119F+E475R+E758R), AaCas12b (Q119F+E475R+E758R+D570A+E848A), and AaCas12b (Q119F+E475R+E758R+D570A+D977A) mediated by sgRNA1 and sgRNA2 specifically recognizing target sites in HBG1/2. Control sgRNA not targeting any sequence of HBG1/2 served as negative control.

FIG. 12A is schematic drawing of an exemplary construct encoding AaCas12b (Q119F+E475R+E758R+D570A+D977A) fused with KRAB under the control of a CMV promoter, together with an sgRNA under the control of a U6 promoter. FIG. 12B shows the relative mRNA levels of mouse Nav1.7 in mouse N2a cells transfected with AaCas12b (Q119F+E475R+E758R+D570A+D977A)-KRAB fusion proteins targeting different sites of the SCN9A gene meditated by different sgRNAs. No sgRNA transfection served as control.

### DETAILED DESCRIPTION

The present application provides engineered Cas12b nucleases with increased enzymatic activities, such as gene editing activity, by introducing one, two, or three types of mutations with respect to a reference Cas12b nuclease. Also provided are engineered Cas12b nucleases or effector proteins thereof with reduced or abolished nuclease activity (e.g., dCas12b). Also provided are engineered guide RNAs (gRNAs) with engineered scaffold sequences, which when used together with Cas12b nucleases (wildtype or engineered), can increase Cas12b enzymatic activities (e.g., gene editing activity). Engineered Cas12b effector proteins, methods of using the engineered Cas12b nucleases or the engineered Cas12b effector proteins, and/or the engineered gRNAs are also provided.

### I. Definitions

Unless defined otherwise, all technical and scientific terms used herein have the same meaning as is commonly understood by one of ordinary skill in the art to which this disclosure belongs.

As used herein, the term “Cas12b protein” is used in its broadest sense and includes parental or reference Cas12b proteins (e.g., AaCas12b protein comprising SEQ ID NO: 1), derivatives or variants thereof (e.g., engineered Cas12b, dCas12b, or engineered Cas12b effector protein), and functional fragments such as oligonucleotide-binding fragments thereof.

As used herein, an “effector protein” refers to a protein having an activity, such as site-specific binding activity, single-strand DNA cleavage or editing activity, double-strand DNA cleavage or editing activity, single-strand RNA cleavage or editing activity, or transcriptional regulation activity.

As used herein, “guide RNA” and “gRNA” are used herein interchangeably to refer to RNA that is capable of forming a complex with a Cas12b nuclease or effector protein and a target nucleic acid (e.g., duplex DNA). A guide RNA may comprise a single RNA molecule or two or more RNA molecules associated with each other via hybridization of complementary regions in the two or more RNA molecules. When used in connection with a dual RNA-guided Cas nuclease, such as Cas12b, a guide RNA comprises a crRNA and a tracrRNA, or a single guide RNA (sgRNA). The “crRNA” or “CRISPR RNA” comprises a guide sequence that has sufficient complementarity to a target sequence of a target nucleic acid (e.g., duplex DNA), which guides sequence-specific binding of the CRISPR complex to the target nucleic acid. The “tracrRNA” or “trans-activating CRISPR RNA” is partially complementary to and base pairs with the crRNA, and may play a role in the maturation of the crRNA. A “single guide RNA” or “sgRNA” is an engineered guide RNA having both crRNA and tracrRNA fused to each other in a single molecule.

As used herein, the term “CRISPR array” refers to a nucleic acid (e.g., DNA) fragment comprising CRISPR repeats and spacers, which begins from the first nucleotide of the first CRISPR repeat and ends at the last nucleotide of the last (terminal) CRISPR repeat. Typically, each spacer in the CRISPR array is located between two repeats. As used herein, the term “CRISPR repeat” or “CRISPR direct repeat” or “direct repeat” refers to a plurality of short direct repeat sequences that exhibit very little or no sequence variation in a CRISPR array. Appropriately, V-I direct repeats may form a stem-loop structure.

As used herein, “donor template nucleic acid” or “donor template” is used interchangeably to refer to a nucleic acid molecule that can be used by one or more cell proteins to alter the structure of a target nucleic acid after the CRISPR enzyme described herein alters the target nucleic acid. In some examples, the donor template nucleic acid is a double-stranded nucleic acid. In some examples, the donor template nucleic acid is a single-stranded nucleic acid. In some examples, the donor template nucleic acid is linear. In some examples, the donor template nucleic acid is circular (e.g., plasmid). In some examples, the donor template nucleic acid is an exogenous nucleic acid molecule. In some examples, the donor template nucleic acid is an endogenous nucleic acid molecule (e.g., chromosome).

The terms “nucleic acid,” “polynucleotide,” and “nucleotide sequence” are used interchangeably to refer to a polymeric form of nucleotides of any length, including deoxyribonucleotides, ribonucleotides, combinations thereof, and analogs thereof. “Oligonucleotide” and “oligo” are used interchangeably to refer to a short polynucleotide, having no more than about 50 nucleotides.

As used herein, “complementarity” refers to the ability of a nucleic acid to form hydrogen bond(s) with another nucleic acid by traditional Watson-Crick base-pairing. A percent complementarity indicates the percentage of residues in a nucleic acid molecule which can form hydrogen bonds (i.e., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence form hydrogen bonds with the same number of contiguous residues in a second nucleic acid sequence. “Substantially complementary” as used herein refers to a degree of complementarity that is at least about any one of 70%, 75%, 80%, 85%, 90%, 95%, 97%, 98%, 99%, or 100% over a region of about 40, 50, 60, 70, 80, 100, 150, 200, 250 or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay,” Elsevier, N,Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. A sequence capable of hybridizing with a given sequence is referred to as the “complement” of the given sequence.

“Percentage (%) sequence identity” with respect to a nucleic acid sequence is defined as the percentage of nucleotides in a candidate sequence that are identical with the nucleotides in the specific nucleic acid sequence, after aligning the sequences by allowing gaps, if necessary, to achieve the maximum percent sequence identity. “Percentage (%) sequence identity” with respect to a peptide, polypeptide or protein sequence is the percentage of amino acid residues in a candidate sequence that are identical substitutions to amino acid residues in the specific peptide or amino acid sequence, after aligning the sequences by allowing gaps, if necessary, to achieve the maximum percent sequence homology. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The terms “polypeptide”, and “peptide” are used interchangeably herein to refer to polymers of amino acids of any length. The polymer may he linear or branched, it may comprise modified amino acids, and it may be interrupted by non-amino acids. A protein may have one or more polypeptides. The terms also encompass an amino acid polymer that has been modified; for example, disulfide bond formation, glycosylation, lipidation, acetylation, phosphorylation, or any other manipulation, such as conjugation with a labeling component.

As used herein, a “variant” is interpreted to mean a polynucleotide or polypeptide that differs from a reference polynucleotide or polypeptide, respectively, but retains essential properties. A typical variant of a polynucleotide differs in nucleic acid sequence from another, reference polynucleotide. Changes in the nucleic acid sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein, the term “wild-type” has the meaning commonly understood by those skilled in the art to mean a typical form of an organism, a strain, a gene, or a feature that distinguishes it from a mutant or variant when it exists in nature. It can be isolated from sources in nature and not intentionally modified.

As used herein, the terms “non-naturally occurring” or “engineered” are used interchangeably and refer to artificial participation. When these terms are used to describe a nucleic acid molecule or polypeptide, it is meant that the nucleic acid molecule or polypeptide is at least substantially freed from at least one other component of its association in nature or as found in nature.

As used herein, the term “orthologue”, or “ortholog” has the meaning as commonly understood by one of ordinary skill in the art. As a further guide, an “orthologue” of a protein as referred to herein refers to a protein belonging to a different species that performs the same or similar function as a protein that is an orthologue thereof.

As used herein, the term “identity” is used to mean the matching of sequences between two polypeptides or between two nucleic acids. When a position in the two sequences being compared is occupied by the same base or amino acid monomer subunit (for example, a position in each of the two DNA molecules is occupied by adenine, or two Each position in each of the polypeptides is occupied by lysine, and then each molecule is identical at that position. The “percent identity” between the two sequences is a function of the number of matching positions shared by the two sequences divided by the number of positions to be compared×100. For example, if 6 of the 10 positions of the two sequences match, then the two sequences have 60% identity. For example, the DNA sequences CTGACT and CAGGTT share 50% identity (3 out of a total of 6 positions match). Typically, the comparison is made when the two sequences are aligned to produce maximum identity. Such alignment can be achieved by, for example, the method of Needleman et al. (1970) J. Mol. Biol. 48: 443-453, which can be conveniently performed by a computer program such as the Align program (DNAstar, Inc.). It is also possible to use the algorithm of E. Meyers and W. Miller (Comput. Appl Biosci., 4: 11-17 (1988)) integrated into the ALIGN program (version 2.0), using the PAM 120 weight residue table. The gap length penalty of 12 and the gap penalty of 4 were used to determine the percent identity between the two amino acid sequences. In addition, the Needleman and Wunsch (J MoI Biol. 48: 444-453 (1970)) algorithms in the GAP program integrated into the GCG software package (available at www.gcg.com) can be used, using the Blossum 62 matrix or The PAM250 matrix and the gap weight of 16, 14, 12, 10, 8, 6 or 4 and the length weight of 1, 2, 3, 4, 5 or 6 to determine the percent identity between two amino acid sequences.

A “cell” as used herein, is understood to refer not only to the particular individual cell, but to the progeny or potential progeny of the cell. Because certain modifications may occur in succeeding generations due to either mutation or environmental influences, such progeny may not, in fact, be identical to the parent cell, but are still included within the scope of the term as used herein.

The term “transduction” and “transfection” as used herein include all methods known in the art using an infectious agent (such as a virus) or other means to introduce DNA into cells for expression of a protein or molecule of interest. Besides a virus or virus like agent, there are chemical-based transfection methods, such as those using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, delivery of plasmids, or transposons; particle-based methods, such as using a gene gun, magnectofection or magnet assisted transfection, particle bombardment; and hybrid methods, such as nucleofection.

The term “transfected” or “transformed” or “transduced” as used herein refers to a process by which exogenous nucleic acid is transferred or introduced into the host cell. A “transfected” or “transformed” or “transduced” cell is one, which has been transfected, transformed or transduced with exogenous nucleic acid.

The term “in vivo” refers to inside the body of the organism from which the cell is obtained. “Ex vivo” or “in vitro” means outside the body of the organism from which the cell is obtained.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

The term “effective amount” used herein refers to an amount of a compound or composition sufficient to treat a specified disorder, condition or disease such as ameliorate, palliate, lessen, and/or delay one or more of its symptoms. As is understood in the art, an “effective amount” may be in one or more doses, i.e., a single dose or multiple doses may be required to achieve the desired treatment endpoint.

A “subject,” an “individual,” or a “patient” are used herein interchangeably for purposes of treatment, and refers to any animal, such as a mammal (including humans, domestic and farm animals, and zoo, sports, or pet animals, such as dogs, horses, cats, hamsters, guinea pigs, rabbits, monkeys, sheep, cows, etc.), a bird, a reptile, a fish, etc. In some embodiments, the individual is a human individual.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X.”

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

### II. Cas12b Nucleases and Effector Proteins

The present application provides engineered Cas12b nucleases and effector proteins that have improved activity, such as target binding, double-strand cleavage activity, nickase activity, and/or gene-editing activity. Also provided are engineered Cas12b nucleases with reduced or abolished nuclease activity (dCas12b). In some embodiments, there is provided an engineered Cas12b effector protein (e.g., Cas12b nuclease, Cas12b nickase, Cas12b fusion effector protein, or split Cas12b effector protein) comprising any one of the engineered Cas12b nucleases described herein or a functional derivative thereof.

### Engineered Cas12b Nuclease

The present application in one aspect provides engineered Cas12b effector proteins that have improved activity (e.g., target binding, double-strand cleavage activity, nickase activity, and/or gene-editing activity).

In some embodiments, there is provided an engineered Cas12b nuclease, comprising one, two, or three types of mutations with respect to a reference Cas12b nuclease, wherein the mutations comprise: (1) substitution of one or more amino acid residues in the reference Cas12b nuclease that interact with a protospacer adjacent motif (PAM) with a positively charged amino acid residue (e.g., R, H, K); and/or (2) substitution of one or more amino acid residues in the reference Cas12b nuclease that are involved in opening DNA double strands (dsDNA) with an amino acid residue having an aromatic ring (e.g., F, Y, W); and/or (3) substitution of one or more amino acid residues in the RuvC domain of the reference Cas12b nuclease that interact with a single-stranded DNA substrate with a positively charged amino acid residue (e.g., R, H, K) or a hydrophobic amino acid residue (e.g., F, Y, W, M). In some embodiments, the reference Cas12b nuclease is a naturally occurring wild-type Cas12b nuclease. In some embodiments, the reference Cas12b nuclease is a natural variant Cas12b nuclease. In some embodiments, the reference Cas12b nuclease is a Cas12b nuclease from Alicyclobacillus acidiphilus (AaCas12b). In some embodiments, the reference Cas12b nuclease comprises the amino acid sequence of SEQ ID NO: 1. In some embodiments, the engineered Cas12b nuclease has increased activity (e.g., increasing at least about any of 10%, 20%, 30%, 40%, 50%, 60%, 70%, 80%, 90%, 1-fold, 1.2-fold, 1.5-fold, 2-fold, 5-fold, 10-fold, 20-fold, 50-fold, 100-fold, or higher) (e.g., target binding, double-strand cleavage activity, nickase activity, and/or gene-editing activity) compared to the reference Cas12b nuclease.

The engineered Cas12b nuclease may comprise one or more of the mutations described below in sections A-C below. In some embodiments, the one or more of the mutations in the present application may be combined with any one of the known Cas12b mutations, such as the mutations described in section D below, to produce engineered Cas12b nucleases of improved activity.

In some embodiments, there is provided an engineered Cas12b nuclease comprising one or more mutations with respect to a reference Cas12b nuclease, wherein the one or more mutations comprise substitution of one or more amino acid residues in the reference Cas12b nuclease that interact with a PAM with a positively charged amino acid residue (e.g., R or K). In some embodiments, there is provided an engineered Cas12b nuclease comprising one or more mutations with respect to a reference Cas12b nuclease, wherein the one or more mutations comprises substitution of one or more amino acid residues in the reference Cas12b nuclease that are involved in opening DNA double strands with an amino acid residue having an aromatic ring (e.g., W, Y or F). In some embodiments, there is provided an engineered Cas12b nuclease comprising one or more mutations with respect to a reference Cas12b nuclease, wherein the one or more mutations comprise substitution of one of more amino acid residues in the RuvC domain of the reference Cas12b nuclease that interact with a single-stranded DNA substrate with a positively charged amino acid residue (e.g., R or K). In some embodiments, there is provided an engineered Cas12b nuclease comprising one or more mutations with respect to a reference Cas12b nuclease, wherein one or more mutations comprise substitution of one of more amino acid residues in the RuvC domain of the reference Cas12b nuclease that interact with a single-stranded DNA substrate with a hydrophobic amino acid residue (e.g., W, Y, F or M). In some embodiments, the reference Cas12b nuclease comprises the amino acid sequence of SEQ ID NO: 1.

In some embodiments, there is provided an engineered Cas12b nuclease comprising one or more mutations with respect to a reference Cas12b nuclease, wherein the one or more mutations comprise: 1) substitution of one or more amino acid residues in the reference Cas12b nuclease that interact with PAM with a positively charged amino acid residue (e.g., R, H, K), and 2) substitution of one or more amino acid residues in the reference Cas12b nuclease that are involved in opening DNA double strands with an amino acid residue having an aromatic ring (e.g., F, Y, W). In some embodiments, there is provided an engineered Cas12b nuclease comprising one or more mutations with respect to a reference Cas12b nuclease, wherein the one or more mutations comprise: 1) substitution of one or more amino acid residues in the reference Cas12b nuclease that interact with PAM with a positively charged amino acid residue (e.g., R, H, K), and 2) substitution of one of more amino acid residues in the RuvC domain of the reference Cas12b nuclease that interact with a single-stranded DNA substrate with a positively charged amino acid residue (e.g., R, H, K) or a hydrophobic amino acid residue (e.g., F, Y, W, M). In some embodiments, there is provided an engineered Cas12b nuclease comprising one or more mutations with respect to a reference Cas12b nuclease, wherein the one or more mutations comprise: 1) substitution of one or more amino acid residues in the reference Cas12b nuclease that are involved in opening DNA double strands with an amino acid residue having an aromatic ring (e.g., F, Y, W), and 2) substitution of one of more amino acid residues in the RuvC domain of the reference Cas12b nuclease that interact with a single-stranded DNA substrate with a positively charged amino acid residue (e.g., R, H, K) or a hydrophobic amino acid residue (e.g., F, Y, W, M). In some embodiments, the reference Cas12b nuclease comprises the amino acid sequence of SEQ ID NO: 1.

In some embodiments, there is provided an engineered Cas12b nuclease comprising one or more mutations with respect to a reference Cas12b nuclease, wherein the one or more mutations comprise: 1) substitution of one or more amino acid residues in the reference Cas12b nuclease that interact with PAM with a positively charged amino acid residue (e.g., R, H, K), 2) substitution of one or more amino acid residues in the reference Cas12b nuclease that are involved in opening DNA double strands with an amino acid residue having an aromatic ring (e.g., F, Y, W), and 3) substitution of one of more amino acid residues in the RuvC domain of the reference Cas12b nuclease that interact with a single-stranded DNA substrate with a positively charged amino acid residue (e.g., R, H, K). In some embodiments, the reference Cas12b nuclease comprises the amino acid sequence of SEQ ID NO: 1.

In some embodiments, there is provided an engineered Cas12b nuclease comprising one or more mutations with respect to a reference Cas12b nuclease, wherein the one or more mutations comprise: 1) substitution of one or more amino acid residues in the reference Cas12b nuclease that interact with PAM with a positively charged amino acid residue (e.g., R, H, K), 2) substitution of one or more amino acid residues in the reference Cas12b nuclease that are involved in opening DNA double strands with an amino acid residue having an aromatic ring (e.g., F, Y, W), and 3) substitution of one of more amino acid residues in the RuvC domain of the reference Cas12b nuclease that interact with a single-stranded DNA substrate with a hydrophobic amino acid residue (e.g., F, Y, W, M). In some embodiments, the reference Cas12b nuclease comprises the amino acid sequence of SEQ ID NO: 1.

The mutations described herein may be designed based on the structure of the reference Cas12b nucleases. Crystal structures of Alicyclobacillus acidoterrestris Cas12b bound to sgRNA as a binary complex and to target DNAs as ternary complexes have been described in Yang H., et al. Cell 167:1814-1828(2016) and Liu L. et al. Mol. Cell 65:310-322(2017). Briefly, the crystal structures show 2 discontinuous REC (recognition, residues 15-386, 658-783) and NUC (nuclease, residues 1-14, 387-658 and 784-1129) lobes composed of several domains each. The crRNA (or single guide RNA, sgRNA) binds in a central channel between the two lobes. PAM recognition is sequence specific and occurs mostly via interaction with the REC1 (helical-1) and WED-II (OBD-II) domains. The sgRNA-target DNA heteroduplex binds primarily to the REC lobe in a sequence-independent manner.

It is understood that other Cas12b orthologues, such as BhCas12b (SEQ ID NO: 59), Bs3Cas12b (SEQ ID NO: 56), LsCas12b (SEQ ID NO: 58), SbCas12b (SEQ ID NO: 60), AkCas12b (SEQ ID NO: 54), AmCas12b (SEQ ID NO: 55), BsCas12b (SEQ ID NO: 57), and DiCas12b etc., have similar domain structures as AaCas12b (SEQ ID NO: 1) and other exemplary reference Cas12b proteins described herein, and the engineered Cas12b proteins may be designed based on any one of the orthologues using split positions that correspond to the exemplary engineered AaCas12b proteins described herein. Corresponding positions refer to the positions in two polypeptides that are aligned with each other when the amino acid sequences of the two polypeptides are aligned with each other. See, FIG. 8 of the present application. Also FIG. S2 of Teng F. et al., Cell Discovery (2019) 5:23 provides an alignment of AaCas12b, AkCas12b, AmCas12b, Bs3Cas12b, BsCas12b, LsCas12b, BhCas12b and SbCas12b, which is incorporated herein by reference in its entirety.

A. Substitution of One or More Amino Acid Residues in the Reference Cas12b that Interact with PAM with a Positively Charged Amino Acid Residue.

In some embodiments, the engineered Cas12b nuclease comprises a substitution of one or more amino acid residues in the reference Cas12b nuclease that interact with a PAM with a positively charged amino acid residue (e.g., R, H, K). In some embodiments, the engineered Cas12b nuclease comprises one, two, three, four, five, or six amino acid substitutions.

In some embodiments, the one or more amino acid residues in the reference Cas12b nuclease that interact with PAM are amino acids within 15 (e.g., within any of 14, 13, 12, 11, 10, 9, 8, 7, 6, 5, 4, 3, 2, 1, or less) angstroms from PAM in a three-dimensional structure. In some embodiments, the one or more amino acid residues in the reference Cas12b nuclease that interact with PAM are amino acids within 10 angstroms from PAM in a three-dimensional structure. In some embodiments, the one or more amino acid residues in the reference Cas12b nuclease that interact with PAM are amino acids within 9 angstroms from PAM in a three-dimensional structure. In some embodiments, the one or more amino acid residues that interact with PAM are in one or more of the following positions: 116, 123, 130, 132, 144, 145, 153, 173, 222, 395, 400, and 475. In some embodiments, the one or more amino acid residues that interact with PAM comprise one or more of the following amino acid residues: D116, K123, D130, D132, N144, K145, E153, D173, Q222, D395, N400, and E475. In some embodiments, the one or more amino acid residues that interact with PAM comprise one or more of the following amino acid residues: D116 and E475. In some embodiments, the amino acid residue numbering is according to SEQ ID NO: 1.

In the context of the present application, D116 refers to the 116^{th} amino acid D (Aspartic acid) in the referenced amino acid sequence. The 3-letter and 1-letter abbreviations of the commonly used amino acid are listed as follows:

| | | | |
|---|---|---|---|
| Ala (A) | Leu (L) | Gln (Q) | Ser (S) |
| Arg (R) | Lys (K) | Glu (E) | Thr (T) |
| Asn (N) | Met (M) | Gly (G) | Trp (W) |
| Asp (D) | Phe (F) | His (H) | Tyr (Y) |
| Cys (C) | Pro (P) | Ile (I) | Val (V) |

As used herein, “the amino acid is at position X, wherein the amino acid numbering is according to SEQ ID NO: 1” refers to the amino acid residue located at a certain position of the reference enzyme Cas12b, which corresponds to position X in SEQ ID NO: 1, when the amino acid sequence of the reference enzyme Cas12b and SEQ ID NO: 1 are aligned based on sequence homology. For example, FIG. 8 shows a homology alignment of the amino acid sequences of Cas12b orthologues (SEQ ID NOs: 1 and 54-60). A skilled person in the art can readily use known software, such as Clustal Omega, to compare and align the amino acid sequence of any reference Cas12b nuclease against SEQ ID NO: 1 to determine the amino acid position that correspond to position X in SEQ ID NO: 1.

In some embodiments, the positively charged amino acid residue is R, H, or K. In some embodiments, the positively charged amino acid residue is R. In some embodiments, the positively charged amino acid residue is K.

In some embodiments, the substitution of one or more amino acid residues in the reference Cas12b nuclease that interact with PAM with the positively charged amino acid residue are one or more of the following substitutions: D116R, K123R, D130R, D132R, N144R, K145R, E153R, D173R, Q222R, D395R, N400R, and E475R. In some embodiments, the substitution of one or more amino acid residues in the reference Cas12b nuclease that interact with PAM with the positively charged amino acid residue are one or more of the following substitutions: D116R and E475R. In some embodiments, the engineered Cas12b nuclease comprises a D116R mutation. In some embodiments, the engineered Cas12b nuclease comprises an E475R mutation. In some embodiments, the amino acid residue numbering is according to SEQ ID NO: 1.

In some embodiments, the engineered Cas12b nuclease comprises an amino acid sequence having at least about 85% sequence identity, such as at least about any one of 87%, 90%, 91%, 92%, 93%, 94%, 95%, 96%, 97%, 98%, or 99% sequence identity, to the amino acid sequence of SEQ ID NO: 2 or 3. In some embodiments, the engineered Cas12b nuclease comprises the amino acid sequence of SEQ ID NO: 2 or 3.

B. Substitution of One or More Amino Acid Residues in the Reference Cas12b Nuclease that are Involved in Opening DNA Double Strands with an Amino Acid Residue Having an Aromatic Ring

In some embodiments, the engineered Cas12b nuclease comprises substitution of one or more amino acid residues in the reference Cas12b nuclease that are involved in opening the DNA double strands with an amino acid residue having an aromatic ring (e.g., F, Y, W). In some embodiments, the engineered Cas12b nuclease comprises one, two, three, four, five, or six substitutions of the amino acid residues.

In some embodiments, the one or more amino acid residues that are involved in opening the DNA double strands interact with the last base pair in PAM relative to the 3′end of a target strand. For example, the PAM sequence recognized by AaCas12b is 5′-TTN-3′ base pair. The last base pair in the PAM relative to the 3′ end of a target strand is the base pair formed by the N base at the 3′end of the PAM sequence, following which is the sequence of the target site.

In some embodiments, the one or more amino acid residues that are involved in opening the DNA double strands are in one or more of the following positions: 118 and 119, such as Q118 and Q119. In some embodiments, the amino acid residue numbering is according to SEQ ID NO: 1.

In some embodiments, the amino acid residue having an aromatic ring is Y, F, or W. In some embodiments, the amino acid residue involved in opening the DNA double strands is substituted with F, Y or W. In some embodiments, the engineered Cas12b nuclease comprises any of: i) Q118Y, Q118F, or Q118W; and/or ii) Q119Y, Q119F, or Q119W. In some embodiments, the amino acid residue numbering is according to SEQ ID NO: 1.

In some embodiments, the substitution of one or more amino acid residues in the reference Cas12b nuclease that are involved in opening the DNA double strands with the amino acid having with an aromatic ring is Q119Y, Q119F, or Q119W. In some embodiments, the amino acid residue numbering is according to SEQ ID NO: 1.

In some embodiments, the engineered Cas12b nuclease comprises an amino acid sequence having at least about 85% sequence identity, such as at least about any one of 88%, 90%, 91%, 92%, 93%, 94%, 95%, 96%, 97%, 98%, or 99% sequence identity, to the amino acid sequence of SEQ ID NO: 4, 5, or 6. In some embodiments, the engineered Cas12b nuclease comprises the amino acid sequence of SEQ ID NO: 4, 5, or 6.

C. Substitution of One or More Amino Acid Residues in the RuvC Domain of the Reference Cas12b Nuclease that Interact with a Single-Stranded DNA Substrate with a Positively Charged Amino Acid Residue or a Hydrophobic Amino Acid Residue.

In some embodiments, the engineered Cas12b nuclease comprises substitution of one or more amino acid residues in the reference Cas12b nuclease that are in the RuvC domain and interact with a single-stranded DNA substrate with a positively charged amino acid residue (e.g., R, H, K). In some embodiments, the engineered Cas12b nuclease comprises substitution of one or more amino acid residues in the reference Cas12b nuclease that are in the RuvC domain and interact with a single-stranded DNA substrate with a hydrophobic amino acid residue (e.g., F, Y, W, M). In some embodiments, the engineered Cas12b nuclease comprises one, two, three, four, five, or six substitutions of the amino acid residues.

In some embodiments, the one or more amino acid residues that are in the RuvC domain and interact with a single-stranded DNA substrate are within 15 (e.g., within any of 14, 13, 12, 11, 10, 9, 8, 7, 6, 5, 4, 3, 2, 1, or less) angstroms from the single-stranded DNA substrate in a three-dimensional structure. In some embodiments, the one or more amino acid residues that are in the RuvC domain and interact with a single-stranded DNA substrate are within 10 angstroms from the single-stranded DNA substrate in a three-dimensional structure. In some embodiments, the one or more amino acid residues that are in the RuvC domain and interact with a single-stranded DNA substrate are within 9 angstroms from the single-stranded DNA substrate in a three-dimensional structure.

The RuvC domain is the active domain of the Cas12b protein responsible for cutting single-stranded DNA or double-stranded DNA. In the primary sequence of the protein, the RuvC domain comprises a first RuvC domain (RuvC-1), a second RuvC domain (RuvC-II) and a third RuvC domain (RuvC-III).

In some embodiments, the one or more amino acid residues that are in the RuvC domain and interact with the single-stranded DNA substrate are in one or more of the following positions: 300, 301, 304, 329, 636, 639, 647, 682, 757, 758, 761, 764, 768, 852, 854, 856, 857, 858, 860, 862, 863, 865, 866, 867, 869, 938, 956, 957, 958, 994, 1093, and 1097. In some embodiments, the one or more amino acid residues that are in the RuvC domain and interact with the single-stranded DNA substrate comprise one or more of the following amino acid residues: D300, K301, E304, N329, E636, Q639, T647, Q682, I757, E758, E761, E764, K768, E852, Q854, N856, N857, D858, P860, S862, E863, N865, Q866, L867, Q869, E938, E956, G957, E958, I994, Q1093, and W1097. In some embodiments, the one or more amino acid residues that are in the RuvC domain and interact with the single-stranded DNA substrate comprise one or more of the following amino acid residues: D300, K301, E636, Q639, T647, Q682, I757, E758, E761, K768, Q854, N857, D858, N865, Q866, Q869, I994, Q1093, and W1097. In some embodiments, the one or more amino acid residues that are in the RuvC domain and interact with the single-stranded DNA substrate comprise one or more of the following amino acid residues: E636, I757, E758, E761, Q854, N857, D858, N865, Q866, Q869, and Q1093. In some embodiments, the amino acid residue numbering is according to SEQ ID NO: 1.

In some embodiments, the engineered Cas12b nuclease comprises substitution of one or more amino acid residues in the reference Cas12b nuclease that are in the RuvC domain and interact with the single-stranded DNA substrate with a positively charged amino acid residue (e.g., R, H, K). In some embodiments, the positively charged amino acid residue is R. In some embodiments, the positively charged amino acid residue is K. In some embodiments, the engineered Cas12b nuclease comprises one or more of following substitutions: D300R, K301R, E304R, N329R, E636R, Q639R, T647R, Q682R, I757R, E758R, E761R, E764R, K768R, E852R, Q854R, N856R, N857R, D858R, P860R, S862R, E863R, N865R, Q866R, L867R, Q869R, E938R, E956R, G957R, E958R, I994R, Q1093R, W1097R, E636K, Q639K, T647K, Q682K, I757K, E758K, E761K, Q854K, N857K, D858K, N865K, Q866K, I994K, Q1093K, and W1097K, wherein the amino acid residue numbering is according to SEQ ID NO: 1. In some embodiments, the engineered Cas12b nuclease comprises one or more of following substitutions: D300R, K301R, E636R, Q639R, T647R, Q682R, I757R, E758R, E761R, K768R, Q854R, N857R, D858R, N865R, Q866R, I994R, Q1093R, W1097R, E636K, Q639K, T647K, Q682K, I757K, E758K, E761K, Q854K, N857K, D858K, N865K, I994K, Q1093K, and W1097K, wherein the amino acid residue numbering is according to SEQ ID NO: 1. In some embodiments, the engineered Cas12b nuclease comprises one or more of following substitutions: E636R, I757R, E758R, E761R, Q854R, D858R, E636K, I757K, E758K, E761K, Q854K, N857K, and D858K, wherein the amino acid residue numbering is according to SEQ ID NO: 1. In some embodiments, the engineered Cas12b nuclease comprises one or more of following substitutions: E636R, I757R, E758R, E761R, Q854R, and D858R, wherein the amino acid residue numbering is according to SEQ ID NO: 1. In some embodiments, the engineered Cas12b nuclease comprises one or more of following substitutions: E636K, I757K, E758K, E761K, Q854K, N857K, and D858K, wherein the amino acid residue numbering is according to SEQ ID NO: 1.

In some embodiments, the substitution of one or more amino acid residues in the reference Cas12b nuclease that are in the RuvC domain and interact with the single-stranded DNA substrate are one or more of the following substitutions: E636R, I757R, E758R, E761R, Q854R, N857K, and D858R, wherein the amino acid residue numbering is according to SEQ ID NO: 1. In some embodiments, the engineered Cas12b nuclease comprises an amino acid sequence having at least about 85% sequence identity, such as at least about any one of 88%, 90%, 91%, 92%, 93%, 94%, 95%, 96%, 97%, 98%, or 99% sequence identity, to the amino acid sequence of any one of SEQ ID NOs: 7-13. In some embodiments, the engineered Cas12b nuclease comprises the amino acid sequence of any one of SEQ ID NOs: 7-13.

In some embodiments, the engineered Cas12b nuclease comprises substitution of one or more amino acid residues in the reference Cas12b nuclease that are in the RuvC domain and interact with the single-stranded DNA substrate with a hydrophobic amino acid residue. In some embodiments, the hydrophobic amino acid residue is A, M, L, I, V, C, Y, F or W. In some embodiments, the hydrophobic amino acid residue is W, Y, F, or M. In some embodiments, the hydrophobic amino acid residue is W, Y, or M. In some embodiments, the engineered Cas12b nuclease comprises one or more of following substitutions: i) E758W, E758Y, E758F, or E758M, ii) E761W, E761Y, E761F, or E761M, iii) E863W, E863Y, E863F, or E863M, iv) N865W, N865Y, N865F, or N865M, v) Q866W, Q866F, Q866Y, or Q866M, vi) Q869W, Q869Y, Q869F, or Q869M, vii) E956W, E956Y, E956F, or E956M, and viii) Q1093W, Q1093F, Q1093Y, or Q1093M; wherein the amino acid residue numbering is according to SEQ ID NO: 1. In some embodiments, the engineered Cas12b nuclease comprises one or more of following substitutions: i) E758W, E758Y, or E758M, ii) E761Y, iii) N865W, N865F, or N865Y, iv) Q866M, v) Q869M, and vi) Q1093W, Q1093F, Q1093Y, or Q1093M; wherein the amino acid residue numbering is according to SEQ ID NO: 1. In some embodiments, the engineered Cas12b nuclease comprises one or more of following substitutions: i) N865W or N865Y, ii) Q866M, iii) Q869M, and iv) Q1093W or Q1093Y; wherein the amino acid residue numbering is according to SEQ ID NO: 1. In some embodiments, the engineered Cas12b nuclease comprises one or more of following substitutions: 865W, 865Y, 866M, 869M, 1093W, and 1093Y. In some embodiments, the substitution of one or more amino acid residues in the reference Cas12b nuclease that are in the RuvC domain and interact with the single-stranded DNA substrate are one or more of the following substitutions: N865W, N865Y, Q866M, Q869M, Q1093W, and Q1093Y. In some embodiments, the engineered Cas12b nuclease comprises Q866M and Q869M substitutions. In some embodiments, the amino acid residue numbering is according to SEQ ID NO: 1.

In some embodiments, the engineered Cas12b nuclease comprises an amino acid sequence having at least about 85% sequence identity, such as at least about any one of 88%, 90%, 91%, 92%, 93%, 94%, 95%, 96%, 97%, 98%, or 99% sequence identity, to the amino acid sequence of any one of SEQ ID NOs: 14-20. In some embodiments, the engineered Cas12b nuclease comprises the amino acid sequence of any one of SEQ ID NOs: 14-20.

### D. Other Mutations

Any one or more of the mutations described in sections A-C above can be combined with any one or more of known mutations that increase Cas12b activity, such as target binding, target specificity, double-strand cleavage activity, nickase activity, and/or gene editing activity. Exemplary mutations can be found, for example, in the following documents WO2022120520, WO2022040909, WO2022042557, CN113308451A, and CN112195164A, the contents of which are incorporated herein by reference in their entirety.

In some embodiments, the reference Cas12b protein comprises from the N-terminus to the C-terminus one or more of: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III), and a second Nuc domain (Nuc-II). In some embodiments, other one or more mutations (e.g., insertion, deletion, substitution) can reside in one or more of such domains.

In some embodiments, the engineered Cas12b nuclease further comprises one or more flexible region mutations that increase the flexibility of the flexible region in the reference Cas12b nuclease. The flexible region in the reference Cas12b nuclease can be determined using any method known in the art. In some embodiments, multiple flexible regions are determined based solely on the amino acid sequence of the reference enzyme. In some embodiments, multiple flexible regions are determined based on the structural information of the reference enzyme, including, for example, secondary structure, crystal structure, NMR structure, and the like.

In some embodiments, multiple flexible zones are determined using a program selected from the group: PredyFlexy, FoldUnfold, PROFbval, Flexserv, FlexPred, DynaMine, and Disomine. In some embodiments, the plurality of flexible regions are located at random crimps. In some embodiments, the multiple flexible regions are in the DNA and/or RNA interaction domain of the reference Cas12b nuclease. In some embodiments, the length of the flexible region is at least about 5 (e.g., 5) amino acids.

In some embodiments, the engineered Cas12b nuclease comprises one or more mutations that increase flexibility of a flexible region that corresponds to amino acid residues 855 to 859, wherein the amino acid residue numbering is based on SEQ ID NO: 1, wherein the engineered Cas12b nuclease has an increased activity (e.g., increasing at least about any of 10%, 20%, 30%, 40%, 50%, 60%, 70%, 80%, 90%, 1-fold, 1.2-fold, 1.5-fold, 2-fold, 5-fold, 10-fold, 20-fold, 50-fold, 100-fold, or higher) (e.g., target binding, double-strand cleavage activity, nickase activity, and/or gene-editing activity) compared to a reference Cas12b nuclease. In some embodiments, the reference Cas12b nuclease is AaCas12b. In some embodiments, the reference Cas12b nuclease comprises the amino acid sequence of SEQ ID NO: 1. In some embodiments, the one or more mutations comprise inserting one or more (e.g., 2) G residues in the flexible region. In some embodiments, the one or more G residues are inserted at the N-terminus of a flexible amino acid residue in the flexible region, wherein the flexible amino acid residue is selected from the group consisting of G, S, N, D, H, M, T, E, Q, K, R, A and P. In some embodiments, the flexible amino acid residue is chosen according to the preference: G>S>N>D>H>M>T>E>Q>K>R>A>P. In some embodiments, the one or more mutations comprise substituting a hydrophobic amino acid residue in a flexible region with a G residue, wherein the hydrophobic amino acid residues is selected from the group consisting of L, I, V, C, Y, F, and W. In some embodiments, the one or more mutations that increase flexibility comprises N856G.

### E. Combinations of Mutations

The engineered enzymes obtained from methods described in Sections A-D in this specification in combination with multiple amino acid substitutions in the Exemplary Sequences Table are all within the scope of the present application. In some embodiments, the engineered Cas12b nuclease comprises one or more mutations (e.g., substitutions) described in Sections A-D above.

In some embodiments, the engineered Cas12b nuclease comprises substitutions or a combination of substitutions at any one of the following amino acid residue positions: (1) 116; (2) 475; (3) 119 and 475; (4) 119, 475, and 758; (5) 119; (6) 636; (7) 757; (8) 758; (9) 761; (10) 768; (11) 858; (12) 854; (13) 857; (14) 119, 475, and 758; (15) 768; (16) 757 and 758; (17) 757 and 761; (18) 757 and 768; (19) 758 and 761; (20) 758 and 768; (21) 761 and 768; (22) 757, 758, and 761; (23) 757, 758, and 768; (24) 757, 761 and 768; (25) 758, 761, and 768; (26) 757, 758, 761, and 768; (27) 865; (28) 866; (29) 869; (30) 1093; and (31) 866 and 869, wherein the amino acid position number is in reference to SEQ ID NO: 1.

In some embodiments, the engineered Cas12b nuclease comprises substitutions or a combination of substitutions at any one of the following amino acid residues: (1) D116; (2) E475; (3) Q119 and E475; (4) Q119, E475, and E758; (5) Q119; (6) E636; (7) I757; (8) E758; (9) E761; (10) K768; (11) D858; (12) Q854; (13) N857; (14) Q119, E475, and E758; (15) K768; (16) I757 and E758; (17) I757 and E761; (18) I757 and K768; (19) E758 and E761; (20) E758 and K768; (21) E761 and K768; (22) I757, E758, and E761; (23) I757, E758, and K768; (24) I757, E761 and K768; (25) E758, E761, and K768; (26) I757, E758, E761, and K768; (27) N865; (28) Q866; (29) Q869; (30) Q1093; and (31) Q866 and Q869; wherein the amino acid position number is in reference to SEQ ID NO: 1. In some embodiments, the engineered Cas12b nuclease comprises substitutions or a combination of substitutions at any one of the following amino acid residues: (1) Q866+Q869; (2) Q119+E475; and (3) Q119+E475+E758; and wherein the amino acid residue numbering is according to SEQ ID NO: 1. In some embodiments, the substitution at amino acid positions D116 and/or E475 is substitution with a positively charged amino acid residue, such as R or K. In some embodiments, the substitution at amino acid positions Q119 is substitution with an amino acid residue having an aromatic side chain, such as Y, F, or W. In some embodiments, the substitution at amino acid position E636, I757, E758, E761, K768, Q854, D858, and/or N857 is substitution with a positively charged amino acid residue, such as R or K. In some embodiments, the substitution at amino acid positions N865, Q866, Q869, and/or Q1093 is substitution with a hydrophobic amino acid residues, such as W, Y, or M.

In some embodiments, the engineered Cas12b nuclease comprises any one or more of the following amino acid residues or combinations thereof: (1) 116R; (2) 475R; (3) 119F and 475R; (4) 119F, 475R, and 758R; (5) 119Y; (6) 119F; (7) 119W; (8) 636R; (9) 757R; (10) 758R; (11) 761R; (12) 854R; (13) 857K; (14) 768R; (15) 757R and 758R; (16) 757R and 761R; (17) 757R and 768R; (18) 758R and 761R; (19) 758R and 768R; (20) 761R and 768R; (21) 757R, 758R, and 761R; (22) 757R, 758R, and 768R; (23) 757R, 761R, and 768R; (24) 758R, 761R, and 768R; (25) 757R, 758R, 761R, and 768R; (26) 865W; (27) 865Y; (28) 866M; (29) 869M; (30) 1093W; (31) 1093Y; (32) 866M and 869M; and (33) 858R; wherein the amino acid position number is in reference to SEQ ID NO: 1.

In some embodiments, the engineered Cas12b nuclease comprises any one of the following substitutions or combinations thereof: (1) D116R; (2) E475R; (3) Q119F+E475R; (4) Q119F+E475R+E758R; (5) Q119Y; (6) Q119F; (7) Q119W; (8) I757R; (9) E758R; (10) E761R; (11) K768R; (12) I757R+E758R; (13) I757R+E761R; (14) I757R+K768R; (15) E758R+E761R; (16) E758R+K768R; (17) E761R+K768R; (18) I757R+E758R+E761R; (19) I757R+E758R+K768R; (20) I757R+E761R+K768R; (21) E758R+E761R+K768R; (22) I757R+E758R+E761R+K768R; (23) Q866M; (24) Q869M; (25) Q866M+Q869M; (26) E636R; (27) Q854R; (28) N857K; (29) N865W; (30) N865Y; (31) Q1093W; (32) Q1093Y; and (33) D858R; wherein the amino acid position number is in reference to SEQ ID NO: 1. In some embodiments, the engineered Cas12b nuclease comprises any one of the following substitutions or combinations thereof: (1) Q866M+Q869M; (2) Q119F+E475R; and (3) Q119F+E475R+E758R; and wherein the amino acid residue numbering is according to SEQ ID NO: 1.

In some embodiments, the engineered Cas12b nuclease comprises one or more of following substitutions: D116R, K123R, D130R, D132R, N144R, K145R, E153R, D173R, Q222R, D395R, N400R, and E475R. In some embodiments, the engineered Cas12b nuclease comprises one or more of following substitutions: Q118Y, Q118F, Q118W, Q119Y, Q119F, and Q119W. In some embodiments, the engineered Cas12b nuclease comprises one or more of following substitutions: D300R, K301R, E304R, N329R, E636R, Q639R, T647R, Q682R, I757R, E758R, E761R, E764R, K768R, E852R, Q854R, N856R, N857R, D858R, P860R, S862R, E863R, N865R, Q866R, L867R, Q869R, E938R, E956R, G957R, E958R, I944R, Q1093R, and W1097R. In some embodiments, the engineered Cas12b nuclease comprises one or more of following substitutions: E636K, Q639K, T647K, Q682K, I757K, E758K, E761K, Q854K, N857K, D858K, N865K, Q866K, I994K, Q1093K, and W1097K. In some embodiments, the engineered Cas12b nuclease comprises one or more of following substitutions: E758W, E758Y, E758F, E758M, E761W, E761Y, E761F, E761M, E863W, E863Y, E863F, E863M, N865W, N865Y, N865F, N865M, Q866W, Q866Y, Q866F, Q866M, Q869W, Q869Y, Q869F, Q869M, E956W, E956Y, E956F, E956M, Q1093W, Q1093Y, Q1093F, and Q1093M. In some embodiments, the amino acid position number is in reference to SEQ ID NO: 1.

In some embodiments, the engineered Cas12b nuclease comprises amino acid substitutions at Q866 and Q869. In some embodiments, the engineered Cas12b nuclease comprises amino acid substitutions Q866M and Q869M. In some embodiments, the amino acid position number is in reference to SEQ ID NO: 1. In some embodiments, the engineered Cas12b nuclease comprises an amino acid sequence having at least about 85% sequence identity, such as at least about any one of 88%, 90%, 91%, 92%, 93%, 94%, 95%, 96%, 97%, 98%, or 99% sequence identity, to the amino acid sequence of SEQ ID NO: 20. In some embodiments, the engineered Cas12b nuclease comprises the amino acid sequence of SEQ ID NO: 20.

In some embodiments, the engineered Cas12b nuclease comprises amino acid substitutions at Q119 and E475. In some embodiments, the engineered Cas12b nuclease comprises amino acid substitutions Q119F and E475R. In some embodiments, the amino acid position number is in reference to SEQ ID NO: 1. In some embodiments, the engineered Cas12b nuclease comprises an amino acid sequence having at least about 85% sequence identity, such as at least about any one of 88%, 90%, 91%, 92%, 93%, 94%, 95%, 96%, 97%, 98%, or 99% sequence identity, to the amino acid sequence of SEQ ID NO: 21. In some embodiments, the engineered Cas12b nuclease comprises the amino acid sequence of SEQ ID NO: 21.

In some embodiments, the engineered Cas12b nuclease comprises amino acid substitutions at Q119, E475, and E758. In some embodiments, the engineered Cas12b nuclease comprises amino acid substitutions Q119F, E475R, and E758R. In some embodiments, the amino acid position number is in reference to SEQ ID NO: 1. In some embodiments, the engineered Cas12b nuclease comprises an amino acid sequence having at least about 85% sequence identity, such as at least about any one of 88%, 90%, 91%, 92%, 93%, 94%, 95%, 96%, 97%, 98%, or 99% sequence identity, to the amino acid sequence of SEQ ID NO: 22. In some embodiments, the engineered Cas12b nuclease comprises the amino acid sequence of SEQ ID NO: 22.

### Reference Cas12b Nuclease

In some embodiments, the reference Cas12 nuclease is AaCas12b, or an orthologue thereof. In some embodiments, the reference Cas12b nuclease is a naturally occurring Cas12b nuclease. In some embodiments, the reference Cas12b nuclease is a wild type Cas12b nuclease. In some embodiments, the reference Cas12b nuclease is an engineered Cas12b nuclease.

Cas12b nucleases from various organisms can be used as the reference Cas12b nuclease to provide the engineered Cas12b nuclease and effector protein of the present application. In some embodiments, the reference Cas12b nuclease has enzymatic activity. In some embodiments, the reference Cas12b is a nuclease that cuts two strands of a target double helix nucleic acid (e.g., double helix DNA). In some embodiments, the reference Cas12b is a nickase, which cuts a single strand of a target double helix nucleic acid (e.g., double helix DNA). In some embodiments, the reference Cas12b nuclease is enzymatically inactive (e.g., dCas12b). Orthologues with a certain sequence identity (e.g., at least about any of 60%, 70%, 80%, 85%, 90%, 95%, 98% or more) with Cas12b or its functional derivatives can be used as the basis for designing the engineered Cas12b nuclease or effector protein of this application. In some embodiments, the reference Cas12b nuclease is a mutant Cas12b but does not contain any mutation described in sections A-E above.

In some embodiments, the engineered Cas12b nuclease is based on a functional variant of the naturally occurring Cas12b nuclease. In some embodiments, the functional variant has one or more mutations, such as amino acid substitutions, insertions, and/or deletions. For example, compared with the wild-type naturally occurring Cas12b nuclease, the functional variant may include 1, 2, 3, 4, 5, 6, 7, 8, 9, 10 or more amino acid substitutions. In some embodiments, the one or more substitutions are conservative substitutions. In some embodiments, the functional variant has all the domains of the naturally occurring Cas12b nuclease. In some embodiments, the functional variant does not have one or more domains of the naturally occurring Cas12b nuclease.

Type V-B CRISPR-Cas12b (also known as C2c1) system has been identified as a dual-RNA-guided (i.e., crRNA and tracrRNA) DNA endonuclease system with distinct features from Cas9 and Cas12a (Shmakov, S. et al. Mol. Cell 60, 385-397 (2015)). First, Cas12b was reported to generate staggered ends distal to the PAM site in vitro when reconstituted with the crRNA/tracrRNA duplex. Second, although the RuvC domain of Cas12b is similar to that of Cas9 and Cas12a, its putative Nuc domain shares no sequence or structural similarity to the HNH domain of Cas9 and the Nuc domain of Cas12a. Moreover, Cas12b proteins are smaller than the most widely used SpCas9 and Cas12a (e.g., AacCas12b: 1,129 amino acids (aa); SpCas9: 1,369 aa; AsCas12a: 1,353 aa; LbCas12a: 1,228 aa), making Cas12b suitable for adeno-associated virus (AAV)-mediated in vivo delivery in gene therapy. Compared with small-sized Cas9 proteins, such as SaCas9 and CjCas9, Cas12b recognizes simpler PAM sequences (e.g., AacCas12b: 5′-TTN-3′); compared to SaCas9: 5′-NNGRRT-3′, CjCas9: 5′-NNNNRYAC-3′), which significantly increase the targeting range of Cas12b in the genome. Additionally, Cas12b has minimal off-target effects and thus may serve as a safer choice for therapeutic and clinical applications.

Cas12b (C2c1) nucleases from various organisms may be used as the reference Cas12b nuclease to provide engineered Cas12b effector proteins of the present application. Exemplary Cas12b nucleases have been described, for example, in Shmakov, S. et al. Mol. Cell 60, 385-397 (2015); Shmakov, S. et al. Nat. Rev. Microbiol. 15, 169-182 (2017); WO2016205764, and WO2020087631, the contents of which are incorporated herein by reference in their entirety.

In some embodiments, the engineered Cas12b effector protein is based on a reference Cas12b protein (e.g., Cas12b nuclease) selected from Cas12b proteins from Alicyclobacillus acidiphilus (AaCas12b), Cas12b from Alicyclobacillus kakegawensis (AkCas12b), Cas12b from Alicyclobacillus macrosporangiidus (AmCas12b), Cas12b from Bacillus hisashii (BhCas12b), BsCas12b from Bacillus, Bs3Cas12b from Bacillus, Cas12b from Desulfovibrio inopinatus (DiCas12b), Cas12b from Laceyella sediminis (LsCas12b), Cas12b from Spirochaetes bacterium (SbCas12b), Cas12b from Tuberibacillus calidus (TcCas12b) and functional derivatives thereof. Sequences of naturally occurring Cas12b proteins are known, for example, in UniProtKBl IDs: TOD7A2, A0A6I3SPI6, and AOA6I7FUC4, which are incorporated herein by reference in their entirety.

In some embodiments, the reference Cas12b protein is a Cas12b nuclease from Alicyclobacillus acidiphilus (AaCas12b) or a functional derivative thereof. In some embodiments, the engineered Cas12b effector protein is based on a reference Cas12b protein comprising an amino acid sequence having at least about 85% (e.g., at least about any one of 88%, 90%, 91%, 92%, 93%, 94%, 95%, 96%, 97%, 98%, or 99%) sequence identity to the amino acid sequence of SEQ ID NO: 1. In some embodiments, the engineered Cas12b effector protein is based on a reference Cas12b nuclease comprising the amino acid sequence of SEQ ID NO: 1.

It is noted that orthologues having a certain sequence identity (e.g., at least about any one of 60%, 70%, 80%, 85%, 90%, 95%, 98% or higher) to the reference Cas12b proteins or fragments thereof may be used as basis to design the engineered Cas12b effector proteins of the present application. The skilled artisan can determine, based on the purpose and application, the percentage of sequence identity of an orthologue of Cas12b or fragment thereof suitable for use in the present application. Methods for determining sequence identity values may be found in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Various Cas12b orthologues have been described in WO2020/087631, the content of which is incorporated herein by reference in its entirety. In some embodiments, the engineered Cas12b effector protein is based on a reference Cas12b protein comprising an amino acid sequence having at least about 85% (e.g., at least about any one of 90%, 91%, 92%, 93%, 94%, 95%, 96%, 97%, 98%, or 99%) sequence identity to the amino acid sequence of any one of SEQ ID NOs: 54-60.

### Activity of the Engineered Cas12b

In some embodiments, the engineered Cas12b nuclease has increased activity compared to the reference Cas12b nuclease. In some embodiments, the activity is target DNA binding activity. In some embodiments, the activity is a site-specific nuclease activity. In some embodiments, the activity is double-stranded DNA cleavage activity. In some embodiments, the activity is single-stranded DNA cleavage activity, including, for example, site-specific DNA cleavage activity or non-specific DNA cleavage activity. In some embodiments, the activity is single-stranded RNA cleavage activity, such as site-specific RNA cleavage activity or non-specific RNA cleavage activity. In some embodiments, the activity is measured in vitro. In some embodiments, the activity is measured in cells such as bacterial cells, plant cells, or eukaryotic cells. In some embodiments, the activity is measured in mammalian cells such as rodent cells or human cells. In some embodiments, the activity is measured in human cells such as 293T cells. In some embodiments, the activity is measured in mouse cells, such as Hepa1-6 cells. In some embodiments, compared to the reference Cas12b nuclease, the engineered Cas12b nuclease has at least about any of 10%, 20%, 30%, 40%, 50%, 60%, 70%, 80%, 90%, 1-fold, 1.2-fold, 1.5-fold, 2-fold, 3-fold, 4-fold, 5-fold, 10-fold, 20-fold, 50-fold or more higher activity with respect to the reference Cas12b nuclease. The site-specific nuclease activity of the engineered Cas12b nuclease can be measured using methods known in the art, including, for example, PCR, sequencing, or gel migration assays, as described in the examples provided herein.

In some embodiments, the activity is gene editing activity in a cell. In some embodiments, the cell is a bacterial cell, a plant cell, or an eukaryotic cell. In some embodiments, the cell is a mammalian cell such as a rodent cell or a human cell. In some embodiments, the cell is a 293T cell. In some embodiments, the activity is measured in mouse cells, such as Hepa1-6 cells. In some embodiments, the activity is an indel formation activity at a target genomic site in a cell, such as site-specific cleavage of the target nucleic acid by the engineered Cas12b nuclease and non-homologous end joining (NHEJ) mechanism for DNA repair. In some embodiments, the activity is the insertion of an exogenous nucleic acid sequence at a target genomic site in a cell, for example, site-specific cleavage of the target nucleic acid by the engineered Cas12b nuclease and homologous recombination (HR) mechanism for DNA repair. In some embodiments, the homologous recombination after cleavage by engineered Cas12b nuclease further comprises introducing a donor template. In some embodiments, the engineered Cas12b nuclease has at least about 20% (e.g., at least about any of 30%, 40%, 50%, 60%, 70%, 80%, 90%, 1 time, 1.2 times, 1.5 times, 2 times, 3 times, 4 times, 5 times, 10 times, 20 times, 50 times, or more) increased gene editing activity (e.g., indel formation) at a genomic site of a cell (e.g., human cells such as 293T cells or mouse Hepa1-6 cells) compared to a reference Cas12b nuclease. In some embodiments, the engineered Cas12b nuclease is capable of editing a greater number (e.g., 2, 3, 4, 5, 10, 20, 50, 100, or more) of genomic sites than the reference Cas12b nuclease. In some embodiments, the consensus PAM sequence of the engineered Cas12b nuclease is the same as the reference Cas12b nuclease. In some embodiments, the engineered Cas12b nuclease recognizes more (e.g., 1, 2, 3, 4, 5, 10, 20, 50, 100, or more) PAM sequences compared to the reference Cas12b nuclease.

Any methods known in the art can be used to determine the cleavage or gene editing efficiency of engineered Cas12b nucleases in cells, including, for example, T7 endonuclease 1 (T7E1) determination, PCR, sequencing of target DNA (including, for example, Sanger sequence, and second-generation sequencing), Deletion-tracking insertion and deletion (TIDE) determination or by amplicon analysis for indel detection (IDAA) determination. See, for example, Sentmanat M F et al., “A survey of validation strategies for CRISPR-Cas9 editing,” Scientific Reports, 2018, 8, article number 888, the content of which is incorporated herein by reference in its entirety. In some embodiments, for example, as described in the Examples herein, targeted next-generation sequencing (NGS) is used to measure the gene editing efficiency of the engineered Cas12b nuclease in a cell. Exemplary genomic sites for determining the cleavage or gene editing efficiency of the engineered Cas12b nuclease include, but are not limited to, CCR5, AAVS, CD34, RNF2, SCN9A, HBG1/2, and EMX1. In some embodiments, the engineered Cas12b nuclease can cleave or edit at least about 1, 2, 5, 10, 15, 20, 25, 30, 35, 40, 45, 50, 55, 60, 65, 100, or more loci compared to the average cleavage or gene editing efficiency of a reference Cas12b nuclease in the human cell genome. In some embodiments, the cleavage or gene editing efficiency (e.g., indel rate) of the engineered Cas12b nuclease is at least about any of 10%, 20%, 30%, 40%, 45%, 50%, 55%, 60%, 65%, 70%, 75%, 80%, 85%, 90%, 95%, 1-fold, 2-fold, 5-fold, 10-fold, 20-fold, 50-fold, or higher than a reference Cas12b nuclease.

### Engineered Cas12b Effector Proteins

The present application further provides engineered Cas12b effector proteins based on any one of the engineered Cas12b nucleases, variants (e.g., dCas12b), or functional derivatives described herein. In some embodiments, the engineered Cas12b effector protein comprises (or consists of, or consists essentially of) any one of the engineered Cas12b nucleases, variants, or functional derivatives described herein. In some embodiments, the engineered Cas12b effector protein comprises a functional derivative of the engineered Cas12b nuclease, such as any one of the functional derivatives as described in the section “Functional Derivatives” below.

In some embodiments, the engineered Cas12b effector protein is enzymatically active. In some embodiments, the engineered Cas12b effector protein is a nuclease that cleaves both strands of a target duplex nucleic acid (e.g., duplex DNA). In some embodiments, the engineered Cas12b effector protein is a nickase, i.e., cleaving a single strand of a target duplex nucleic acid (e.g., duplex DNA). In some embodiments, the engineered Cas12b effector protein comprises an enzymatically inactive mutant of the engineered Cas12b nuclease (dCas12b). Mutations at one or more amino acid residues in the active site of a Cas12b nuclease can result in an enzymatically dead Cas12b (dCas12b). For example, D570A, E848A, R785A, E848A, R911A, and/or D977A mutants of AaCas12b (SEQ ID NO: 1) have significantly reduced (e.g., reducing at least about any of 60%, 70%, 80%, 90%, 95%, or more) or no nuclease activities in human cells. See, for example, Teng F. et al., Cell Discovery, 4, Article number: 63 (2018), the content of which is incorporated herein by reference in its entirety. In some embodiments, the engineered Cas12b effector protein comprises an engineered Cas12b having one or more mutations corresponding to D570A, E848A, R785A, E848A, R911A, and D977A of AaCas12b. In some embodiments, one or more mutations selected from the group consisting of D570A, E848A, R785A, E848A, R911A, and D977A is further introduced into AaCas12b comprising Q119F+E475R+E758R mutations. In some embodiments, the enzymatically inactive mutant of the engineered Cas12b nuclease comprises the amino acid sequence of any of SEQ ID NOs: 79-81. In some embodiments, the engineered Cas12b effector protein comprises an engineered Cas12b having a mutation corresponding to the R785A mutation of AaCas12b. In some embodiments, the engineered Cas12b effector protein comprises an engineered Cas12b having a mutation corresponding to the R911A mutation of AaCas12b. In some embodiments, the engineered Cas12b effector protein comprises an engineered Cas12b having a mutation corresponding to the D977A mutation of AaCas12b. In some embodiments, the engineered Cas12b effector protein comprises an engineered Cas12b having a mutation corresponding to the E848A mutation of AaCas12b. In some embodiments, the engineered Cas12b effector protein comprises an engineered Cas12b having a mutation corresponding to the D570A mutation of AaCas12b. In some embodiments, the engineered Cas12b effector protein comprises an engineered Cas12b having a mutation corresponding to the D570A+E848A mutation of AaCas12b, or the D570A+D977A mutation of AaCas12b.

In some embodiments, there is provided an engineered Cas12b nickase. In some embodiments, there is provided an engineered Cas12b fusion effector protein, comprising an engineered Cas12b nuclease or variant or functional derivative thereof (e.g., an enzymatically inactive mutant of the engineered Cas12b nuclease, such as any of SEQ ID NOs: 79-81) fused to a functional domain, such as a translation initiator domain, a transcription repressor domain (e.g., Kruppel associated box (KRAB) domain), a transactivation domain, an epigenetic modification domain, a nucleobase-editing domain (e.g., cytosine base editor (CBE) or adenine base editor (ABE) domain), a reverse transcriptase domain, a reporter domain (e.g., a fluorescent domain), or a nuclease domain (e.g., ZFN domain). In some embodiments, there is provided an engineered Cas12b base editor comprising a catalytically inactive variant of any one of the engineered Cas12b nucleases described herein (e.g., any of SEQ ID NOs: 79-81) fused to a cytosine deaminase domain or an adenosine deaminase domain. In some embodiments, there is provided an engineered Cas12b base editor comprising a catalytically inactive variant of any one of the engineered Cas12b nucleases described herein (e.g., any of SEQ ID NOs: 79-81) fused to a KRAB domain or functional fragment thereof, such as ZIM3 KRAB domain (SEQ ID NO: 72). In some embodiments, there is provided an engineered Cas12b prime editor comprising a catalytically inactive variant of any one of the engineered Cas12b nucleases described herein (e.g., any of SEQ ID NOs: 79-81) fused to a reverse transcriptase domain. In some embodiments, there is provided a split Cas12b effector protein system.

### Variants/Functional Derivatives

The application also provides variants and functional derivatives of any of the engineered Cas12b nucleases described herein. In some embodiments, there is provided an engineered Cas12b effector protein comprising (or consisting of, or consisting essentially of) a functional variant of any of the engineered Cas12b nucleases described herein. In some embodiments, when compared with the amino acid sequence of the corresponding engineered Cas12b nuclease (e.g., any of SEQ ID NOs: 2-22), the amino acid sequence of the functional variant has at least one amino acid residue difference (e.g., has a deletion, insertion, substitution, and/or fusion). In some embodiments, the functional variant has one or more mutations, such as amino acid substitutions, insertions and/or deletions. For example, compared to the engineered Cas12b nuclease, the functional variant may include any one of 1, 2, 3, 4, 5, 6, 7, 8, 9, 10, or more amino acid substitutions. In some embodiments, the one or more substitutions are conservative substitutions. In some embodiments, the functional variant has all the domains of the engineered Cas12b nuclease. In some embodiments, the functional variant does not have one or more domains of the engineered Cas12b nuclease.

For any of the Cas12b variant proteins described herein (e.g., nickase Cas12b protein, inactivated or catalytically inactivated Cas12b (dCas12b), fusion Cas12b), the Cas12b variant may include the same parameters as any of the Cas12b protein sequence described herein (e.g., domains, percent sequence identity, etc.).

Exemplary mutations in Cas12b functional variants are described in WO2016205764, WO2016205749, and WO2020/087631, the contents of each of which are incorporated herein by reference in their entirety.

### Catalytic Activity

In some embodiments, the functional variant has different catalytic activity compared to its non-mutated form of the engineered Cas12b nuclease. In some embodiments, the mutations (e.g., amino acid substitutions, insertions, and/or deletions) are in a catalytic domain of the Cas12b effector protein (e.g., a RuvC domain). In some embodiments, the variant comprises mutations in multiple catalytic domains. A Cas12b effector protein that cleaves one strand but not the other of a double stranded target nucleic acid is referred to herein as a “nickase” (e.g., a “nickase Cas”). In some embodiments, the engineered Cas12b effector protein comprises (or consists of, or consists essentially of) a nickase mutant of the engineered Cas12b nuclease. A Cas12b protein that has substantially no nuclease activity is referred to herein as a dead Cas12b protein (“dCas12b”) (with the caveat that nuclease activity can be provided by a heterologous polypeptide—a fusion partner—in the case of a fusion Cas12b effector protein, which is described in more detail below). In some embodiments, a Cas12b effector protein is considered to substantially lack all DNA cleavage activity when the DNA cleavage activity of the mutated Cas12b is less than about any of 25%, 20%, 10%, 5%, 1%, 0.1%, 0.01%, or lower with respect to its non-mutated form.

In some embodiments, the engineered Cas12b nuclease is a dCas12b. In some embodiments, the engineered Cas12b functional variant comprises a mutation corresponding to D570A of AaCas12b (SEQ ID NO: 1). In some embodiments, the engineered Cas12b functional variant comprises a mutation corresponding to E848A of AaCas12b. In some embodiments, the engineered Cas12b functional variant comprises a mutation corresponding to R785A of AaCas12b. In some embodiments, the engineered Cas12b functional variant comprises a mutation corresponding to E848A of AaCas12b. In some embodiments, the engineered Cas12b functional variant comprises a mutation corresponding to R991A of AaCas12b. In some embodiments, the engineered Cas12b functional variant comprises a mutation corresponding to D977A of AaCas12b. In some embodiments, the engineered Cas12b functional variant comprises a mutation corresponding to D573A of BthCas12b. In some embodiments, the catalytically inactive or substantially inactive variant of AaCas12b (Q119F+E475R+E758R) further comprises one or more substitutions selected form the group consisting of: D570A, E848A, and D977A, wherein the amino acid positions are corresponding to SEQ ID NO: 22. In some embodiments, the dCas12b comprises the amino acid sequence of any of SEQ ID NOs: 79-81.

### Split Cas12b Effector Proteins

The CRISPR-Cas12b systems described herein may comprise any pair of polypeptides (also referred herein as “split Cas12b polypeptides”) comprising split Cas12b portions in this section. Exemplary split Cas12b protein systems have been described, for example, in PCT/CN2020/111057 and PCT/CN2021/114339, the contents of each of which are incorporated herein by reference in their entirety.

In some embodiments, there is provided a split Cas12b effector protein, comprising a first polypeptide comprising an N-terminal portion of any one of the engineered Cas12b nucleases described herein or variant or functional derivative thereof (also referred in this section as “parental Cas12b protein”), and a second polypeptide comprising a C-terminal portion of the engineered Cas12b nuclease or variant or functional derivative thereof, wherein the first polypeptide and the second polypeptide are capable of associating with each other in the presence of a guide RNA comprising a guide sequence to form a CRISPR complex that specifically binds to a target nucleic acid comprising a target sequence complementary to the guide sequence. In some embodiments, the first polypeptide and the second polypeptide each comprises a dimerization domain. In some embodiments, the first dimerization domain and the second dimerization domain associate with each other in the presence of an inducer (e.g., rapamycin). In some embodiments, the first polypeptide and the second polypeptide do not comprise any dimerization domain. In some embodiments, the split Cas12b effector protein is auto-inducing.

The split Cas12b portions are designed based on any one of the engineered Cas12b nucleases described herein, or variants or functional variants thereof.

Cas12b proteins have various structural domains. In some embodiments, a parental Cas12b protein comprises from the N-terminus to the C-terminus: a first WED domain (WED-I; also known as OBD-I domain), a first REC domain (REC1), a second WED domain (WED-II; also known as OBD-II domain), a first RuvC domain (RuvC-I), a bridge helix (BH) domain, a second RuvC domain (RuvC-II), a first Nuc domain (Nuc-I; also known as UK-I domain), a third RuvC domain (RuvC-III) and a second Nuc domain (Nuc-II; also known as UK-II domain). Domain boundaries may be determined using known methods in the art, such as based on crystal structures of a naturally occurring Cas12b protein (e.g., PDB ID Nos: 5U30, 5U31, 5U33, 5U34 and 5WQE for AaCas12b), and/or sequence homology to known functional domains in a parental Cas12b protein. In some embodiments, the AaCas12b has the following domains: WEB-I domain (amino acid residues 1-14), REC1 domain (amino acid residues 15-386), WED-II domain (amino acid residues 387-518), RuvC-I domain (amino acid residues 519-628), BH domain (amino acid residues 629-658), REC2 domain (amino acid residues 659-783), RuvC-II domain (amino acid residues 784-900), Nuc-I domain (amino acid residues 901-974), RuvC-III domain (amino acid residues 975-993), and Nuc-II domain (amino acid residues 994-1129), wherein the amino acid numbering is based on SEQ ID NO: 1.

The engineered Cas12b nuclease or variant or functional derivative thereof is split in the sense that the two split Cas12b portions substantially comprise a functional Cas12b. That Cas12b may function as a genome editing enzyme (when forming a complex with a target DNA and a guide RNA), such as a nuclease that cleaves a single strand or both strands of a duplex nucleic acid, or it may be a catalytically dead-Cas12b (dCas12b), which is essentially a DNA-binding protein with very little or no catalytic activity, due to typically mutation(s) in its catalytic domains. Mutations at one or more amino acid residues in the active site of a reference Cas12b can result in a catalytically dead Cas12b, such as D570A, E848A, R785A, E848A, R911A, and/or D977A mutants of AaCas12b.

The split Cas12b portions described herein can be designed by dividing (i.e., splitting) an engineered Cas12b nuclease or variant or functional derivative thereof (referred herein as “parental Cas12b protein”; such as any of SEQ ID NOs: 2-22 and 79-81) (e.g., a full-length Cas12b protein or a functional variant thereof) into two halves at a split position, which is the point at which the N-terminal portion of the parental Cas12b protein is separated from the C-terminal portion. In some embodiments, the N-terminal portion comprises amino acid residues 1 to X, whilst the C-terminal portion comprises amino acid residues X+1 to the C-terminus end of the parental Cas12b protein. In this example, the numbering is contiguous, but this may not always be necessary as amino acids (or the nucleotides encoding them) could be trimmed from the end of either one of the split ends, and/or mutations (e.g., insertions, deletions and substitutions) at internal regions of the polypeptide chain(s) are also contemplated, provided that sufficient DNA binding activity and, if required, DNA nickase or double-strand cleavage activity, of the reconstituted Cas12b protein is retained, for example at least about any of 30%, 40%, 50%, 60%, 70%, 80%, 90%, 95%, or more activity compared to the parental Cas12b protein.

Also contemplated are split Cas12b portions having some N- and/or C-terminal truncations or deletions, and/or internal mutations with respect to an engineered Cas12b nuclease described herein. A skilled person in the art could readily use the information of the exemplary split Cas12b polypeptides described herein to design counterpart split Cas12b polypeptides based on other Cas12b proteins and functional variants, e.g., by using standard sequence alignment tools.

The split position may be located within a flexible region, such as a loop. Preferably, the split position occurs where an interruption of the amino acid sequence does not result in the partial or full destruction of a structural feature (e.g., alpha-helices or beta-sheets). Unstructured regions (regions that do not show up in the crystal structure because these regions are not structured enough to be “frozen” in a crystal) are often preferred options. It is contemplated that the splits can be made in unstructured regions that are exposed on the surface of a parental Cas12b protein.

In some embodiments, the parental Cas12b protein is not split at or in the vicinity (e.g., within about 10, 8, 6, 5, 4, 3, 2, or 1 amino acid residues) to an amino acid residue involved in interaction with a guide RNA, and/or a target RNA. For example, amino acid residues 4-9, 118-122, 143-144, 442-446, 573-574, 742-746, 753-754, 792-796, 800-819, 835-839, 897-900 and 973-978 of the AaCas12b protein are involved in interaction with a single-guide RNA and/or a target DNA, wherein the numbering is based on SEQ ID NO: 1.

In some embodiments, the parental Cas12b protein is split at an amino acid residue within amino acid residues corresponding to amino acid residues 516 to 793 of the AaCas12b protein, wherein the numbering is based on SEQ ID NO: 1. In some embodiments, the parental Cas12b protein is split at an amino acid residue bordering the WED-II domain and the RuvC-I domain. In some embodiments, the parental Cas12b protein is split at an amino acid residue within amino acid residues corresponding to amino acid residues 516 to 519 of the AaCas12b protein, wherein the numbering is based on SEQ ID NO: 1. In some embodiments, the parental Cas12b protein is split at an amino acid residue bordering the BH domain and the REC2 domain. In some embodiments, the parental Cas12b protein is split at an amino acid residue within amino acid residues corresponding to amino acid residues 621 to 627 of the AaCas12b protein, wherein the numbering is based on SEQ ID NO: 1. In some embodiments, the parental Cas12b protein is split at an amino acid residue bordering the REC2 domain and the RuvC-II domain. In some embodiments, the parental Cas12b protein is split at an amino acid residue within amino acid residues corresponding to amino acid residues 777 to 793 of the AaCas12b protein, wherein the numbering is based on SEQ ID NO: 1. In some embodiments, the parental Cas12b protein is split within the RCE2 domain. In some embodiments, the parental Cas12b protein is split at an amino acid residue within amino acid residues corresponding to amino acid residues 659 to 664, 676 to 684, or 702 to 706 of the AaCas12b protein, wherein the numbering is based on SEQ ID NO: 1.

In some embodiments, the parental Cas12b protein is split at an amino acid residue within no more than about 20 (e.g., no more than about any one of 18, 16, 14, 12, 10, 8, 7, 6, 5, 4, 3, 2, or 1) amino acid residues from an amino acid residue that corresponds to amino acid residue 518 of the AaCas12b protein, wherein the numbering is based on SEQ ID NO: 1. In some embodiments, the parental Cas12b protein is split at an amino acid residue that corresponds to amino acid residue 518 of the AaCas12b protein, wherein the numbering is based on SEQ ID NO: 1. In some embodiments, the parental Cas12b protein is split at an amino acid residue within no more than about 20 (e.g., no more than about any one of 18, 16, 14, 12, 10, 8, 7, 6, 5, 4, 3, 2, or 1) amino acid residues from an amino acid residue that corresponds to amino acid residue 658 of the AaCas12b protein, wherein the numbering is based on SEQ ID NO: 1. In some embodiments, the parental Cas12b protein is split at an amino acid residue that corresponds to amino acid residue 658 of the AaCas12b protein, wherein the numbering is based on SEQ ID NO: 1. In some embodiments, the parental Cas12b protein is split at an amino acid residue within no more than about 20 (e.g., no more than about any one of 18, 16, 14, 12, 10, 8, 7, 6, 5, 4, 3, 2, or 1) amino acid residues from an amino acid residue that corresponds to amino acid residue 783 of the AaCas12b protein, wherein the numbering is based on SEQ ID NO: 1. In some embodiments, the parental Cas12b protein is split at an amino acid residue that corresponds to amino acid residue 783 of the AaCas12b protein, wherein the numbering is based on SEQ ID NO: 1.

In some embodiments, the N-terminal portion of the parental Cas12b protein comprises the WED-I, REC1, WED-II, RuvC-I and BH domains of an AaCas12b protein, and wherein the C-terminal portion of the parental Cas12b protein comprises the REC2, RuvC-II, Nuc-I, RuvC-III and Nuc-II domains of the AaCas12b protein. In some embodiments, the N-terminal portion of the parental Cas12b protein comprises amino acid residues 1 to 658 of the parental Cas12b protein, and the C-terminal portion of the parental Cas12b protein comprises amino acid residues 659 to 1129 of the parental Cas12b protein, wherein the amino acid residue numbering is according to SEQ ID NO: 1.

In some embodiments, the N-terminal portion of the parental Cas12b protein comprises WED-I, REC1, WED-II, RuvC-I, BH and REC2 domains of the parental Cas12b protein, and wherein the C-terminal portion of the parental Cas12b protein comprises RuvC-II, Nuc-I, RuvC-III and Nuc-II domains of the parental Cas12b protein. In some embodiments, the N-terminal portion of the parental Cas12b protein comprises amino acid residues 1 to 783 of the parental Cas12b protein, and the C-terminal portion of the parental Cas12b protein comprises amino acid residues 784 to 1129 of the parental Cas12b protein, wherein the amino acid residue numbering is according to SEQ ID NO. 1.

In some embodiments, the N-terminal portion of the parental Cas12b protein comprises WED-I, REC1, WED-II, RuvC-I and BH domains of the parental Cas12b protein, wherein the C-terminal portion of the parental Cas12b protein comprises RuvC-II, Nuc-I, RuvC-III and Nuc-II domains of the parental Cas12b protein, and wherein REC2 domain of the parental Cas12b protein is split between the N-terminal portion of the parental Cas12b protein and the C-terminal portion of the parental Cas12b protein.

In some embodiments, the N-terminal portion of the parental Cas12b protein comprises WED-I, REC1 and WED-II domains of the parental Cas12b protein, and wherein the C-terminal portion of the parental Cas12b protein comprises RuvC-I, BH, REC2, RuvC-II, Nuc-I, RuvC-III and Nuc-II domains of the parental Cas12b protein. In some embodiments, the N-terminal portion of the parental Cas12b protein comprises amino acid residues 1 to 518 of the parental Cas12b protein, and the C-terminal portion of the parental Cas12b protein comprises amino acid residues 519 to 1129 of the parental Cas12b protein, wherein the amino acid residue numbering is according to SEQ ID NO. 1.

The split point is typically designed in silico and cloned into the constructs. Together, the two split Cas12b portions, the N-terminal and C-terminal parts, form a functional Cas12b protein, comprising preferably at least about 70% or more of the amino acid sequence of the parental Cas12b protein, such as at least about any one of 75%, 80%, 85%, 90%, 95%, 98%, 99% or more of the amino acid sequence of the parental Cas12b protein. Some trimming and mutants are envisioned. Non-functional domains may be removed entirely. For all split Cas12b systems, the two split Cas12b portions may be brought together and that the desired Cas12b function is restored or reconstituted. Activities of the reconstituted Cas12b protein or CRISPR complex (Cas12b+guide RNA complex) can be assessed using known methods in the art. For example, nuclease activity within a cell can be assessed using a T7 endonuclease I (T7EI) assay. Gene-editing activity can also be assessed by DNA sequencing.

In some embodiments, the parental Cas12b protein is split into more than two portions, such as 3, 4, 5, or 6 portions.

The split Cas12b effector proteins may each comprise one or more dimerization domains. In some embodiments, the first polypeptide comprises a first dimerization domain fused to the first split Cas12b effector portion, and the second polypeptide comprises a second dimerization domain fused to the second split Cas12b effector portion. The dimerization domain may be fused to the split Cas12b effector portion via a peptide linker (e.g., a flexible peptide linker such as a GS linker) or a chemical bond. In some embodiments, the dimerization domain is fused to the N-terminus of the split Cas12b effector portion. In some embodiments, the dimerization domain is fused to the C-terminus of the split Cas12b effector portion.

In some embodiments, the split Cas12b effector proteins do not comprise any dimerization domains.

In some embodiments, the dimerization domains promotes association of the two split Cas12b effector portions. In some embodiments, the split Cas12b effector portions are induced to associate or dimerize into a functional Cas12b effector protein by an inducer. In some embodiments, the split Cas12b effector proteins comprise inducible dimerization domains. In some embodiments, the dimerization domains are not inducible dimerization domains, i.e., the dimerization domains dimerize without the presence of an inducer.

An inducer may be an inducing energy source or an inducing molecule other than a guide RNA (e.g., a sgRNA). The inducer acts to reconstitute two split Cas12b effector portions into a functional Cas12b effector protein via induced dimerization of the dimerization domains. In some embodiments, the inducer brings the two split Cas12b effector portions together through the action of induced association of the inducible dimerization domains. In some embodiments, without the inducer, the two split Cas12b effector portions do not associate with each other to reconstitute into a functional Cas12b effector protein. In some embodiments, without the inducer, the two split Cas12b effector portions may associate with each other to reconstitute into a functional Cas12b effector protein in the presence of a guide RNA (e.g., an sgRNA).

The inducer of the present application may be heat, ultrasound, electromagnetic energy or a chemical compound. In some embodiments, the inducer is an antibiotic, a small molecule, a hormone, a hormone derivative, a steroid, or a steroid derivative. In some embodiments, the inducer is abscisic acid (ABA), doxycycline (DOX), cumate, rapamycin, 4-hydroxytamoxifen (4OHT), estrogen or ecdysone. In some embodiments, the split Cas12b effector system is an inducer-controlled system selected from the group consisting of antibiotic based inducible systems, electromagnetic energy based inducible systems, small molecule based inducible systems, nuclear receptor based inducible systems and hormone based inducible systems. In some embodiments, the split Cas12b effector system is an inducer-controlled system is selected from the group consisting of tetracycline (Tet)/DOX inducible systems, light inducible systems, ABA inducible systems, cumate repressor/operator systems, 4OHT/estrogen inducible systems, ecdysone-based inducible systems and FKBP12/FRAP (FKBP12-rapamycin complex) inducible systems. Such inducers are also discussed herein and in PCT/US2013/051418, the content of which is incorporated herein by reference in its entirety. FRB/FKBP/Rapamycin systems have been described in Paulmurugan and Gambhir, Cancer Res, Aug. 15, 2005 65; 7413; and Crabtree et al., Chemistry & Biology 13, 99-107, January 2006, the contents of each of which are incorporated herein by reference in their entirety.

In some embodiments, the pair of split Cas12b effector proteins are separate and inactive until induced dimerization of the dimerization domains (e.g., FRB and FKBP), which results in reassembly of a functional Cas12b effector nuclease. In some embodiments, the first split Cas12b effector protein comprising a first half of an inducible dimer (e.g., FRB) is delivered separately and/or is localized separately from the second split Cas12b effector protein comprising a second half of an inducible dimer (e.g., FKBP).

Other exemplary FKBP-based inducible systems that may be used in inducer-controlled split Cas12b effector systems described herein include, but are not limited to, FKBP which dimerizes with CalcineurinA (CNA), in the presence of FK506; FKBP which dimerizes with CyP-Fas, in the presence of FKCsA; FKBP which dimerizes with FRB, in the presence of Rapamycin; GyrB which dimerizes with GryB, in the presence of Coumermycin; GAI which dimerizes with GID1, in the presence of Gibberellin; or Snap-tag which dimerizes with HaloTag, in the presence of HaXS.

Alternatives within the FKBP family itself are also contemplated. For example, FKBP, which homodimerizes (i.e., one FKBP dimerizes with another FKBP) in the presence of FK1012.

In some embodiments, the dimerization domain is FKBP and the inducer is FK1012. In some embodiments, the dimerization domain is GryB and the inducer is coumermycin. In some embodiments, the dimerization domain is ABA and the inducer is Gibberellin.

In some embodiments, the split Cas12b effector portions may be auto-induced (i.e., auto-activated or self-induced) to associate/dimerize into a functional Cas12b effector protein without the presence of an inducer. Without being bound by any theory or hypothesis, auto-induction of the split Cas12b effector portions may be mediated by binding to a guide RNA, such as an sgRNA. In some embodiments, the first polypeptide and the second polypeptide do not comprise dimerization domains. In some embodiments, the first polypeptide and the second polypeptide comprise dimerization domains.

In some embodiments, the reconstituted Cas12b effector protein of the split Cas12b effector systems described herein (including inducer-controlled and auto-inducible systems) has an editing efficiency of at least about 70% (such as at least about any of 70%, 75%, 80%, 85%, 90%, 95%, 98%, 99% or more efficiency, or 100% efficiency) of the editing efficiency of the parental Cas12b effector protein.

In some embodiments, the reconstituted Cas12b effector protein of an inducer-controlled split Cas12b effector systems described herein has an editing efficiency of no more than about 50% (such as no more than about any of 50%, 45%, 40%, 35%, 30%, 25%, 20%, 15%, 10%, 5%, or less efficiency, or 0% efficiency) without the presence of an inducer (i.e., due to auto-induction) of the editing efficiency of the parental Cas12b effector protein.

### Fusion Cas12b Effector Proteins

The present application also provides engineered Cas12b effector proteins comprising additional protein domains and/or components, such as linkers, nuclear localization/exportation sequences, functional domains, and/or reporter proteins.

In some embodiments, the engineered Cas12b effector protein is a protein complex comprising one or more heterologous protein domains (e.g., about or more than about any of 1, 2, 3, 4, 5, 6, 7, 8, 9, 10, or more domains) in addition to the nucleic acid-targeting domains of the engineered Cas12b nuclease or variant or functional derivative thereof. In some embodiments, the engineered Cas12b effector protein is a fusion protein comprising one or more heterologous protein domains (e.g. about or more than about any of 1, 2, 3, 4, 5, 6, 7, 8, 9, 10, or more domains) fused to the engineered Cas12b nuclease or variant or functional derivative thereof.

In some embodiments, the engineered Cas12b effector proteins of the present application can comprise (e.g., via fusion protein, such as via one or more peptide linkers, for example, GS peptide linkers, etc.) or be associated (e.g., via co-expression of multiple proteins) with one or more functional domains. In some embodiments, the one or more functional domains are enzymatic domains. These functional domains can have various activities, e.g., DNA and/or RNA methylase activity, demethylase activity, transcription activation activity, transcription repression activity, transcription release factor activity, histone modification activity, RNA cleavage activity, DNA cleavage activity, nucleic acid binding activity, and switch activity (e.g., light inducible). In some embodiments, the one or more functional domains are transcriptional activation domains (i.e., transactivation domains) or repressor domains. In some embodiments, the transcriptional activation domain or repressor domain can recruit chromatin modifier(s). In some embodiments, the one or more functional domains are histone-modifying domains. In some embodiments, the one or more functional domains are transposase domains, HR (Homologous Recombination) machinery domains, recombinase domains, and/or integrase domains. In some embodiments, the functional domains are Kruppel associated box (KRAB), VP64, VP16, Fok1, P65, HSF1, MyoD1, biotin-APEX, APOBEC1, AID, PmCDA1, Tad1, and M-MLV reverse transcriptase. In some embodiments, the functional domain is selected from the group consisting of a translation initiator domain, a transcription repressor domain, a transactivation domain, an epigenetic modification domain, a nucleobase-editing domain (e.g., CBE or ABE domain), a reverse transcriptase domain, a reporter domain (e.g., a fluorescent domain) and a nuclease domain. In some embodiments, the functional domain is a KRAB domain, such as KRAB domain of ZIM3. In some embodiments, the KRAB domain comprises the amino acid sequence of SEQ ID NO: 72.

In some embodiments, the positioning of the one or more functional domains in the engineered Cas12b effector proteins allows for correct spatial orientation for the functional domains to affect the target with the attributed functional effects. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., Fok1) is positioned to cleave or partially cleave the target. In some embodiments, the functional domain (e.g., KRAB domain, such as comprising SEQ ID NO: 72) is positioned at the N-terminus of the engineered Cas12b effector protein (e.g., any of SEQ ID NOs: 79-81, such as SEQ ID NO: 81). In some embodiments, the functional domain (e.g., KRAB domain, such as comprising SEQ ID NO: 72) is positioned at the C-terminus of the engineered Cas12b effector protein (e.g., any of SEQ ID NOs: 79-81, such as SEQ ID NO: 81). In some embodiments, the engineered Cas12b effector protein comprises a first functional domain at the N-terminus and a second functional domain at the C-terminus. In some embodiments, the engineered Cas12b effector protein comprises a catalytically inactive mutant (e.g., any of SEQ ID NOs: 79-81) of any one of the engineered Cas12b nucleases described herein fused to one or more functional domains (e.g., KRAB domain).

In some embodiments, the engineered Cas12b effector protein is a transcriptional activator. In some embodiments, the engineered Cas12b effector protein comprises an enzymatically inactive variant (e.g., any of SEQ ID NOs: 79-81) of any one of the engineered Cas12b nucleases described herein fused to a transactivation domain. In some embodiments, the transactivation domain is selected from the group consisting of VP64, p65, HSF1, VP16, MyoD1, HSF1, RTA, SET7/9, and combinations thereof. In some embodiments, the transactivation domain comprises VP64, p65 and HSF1. In some embodiments, the engineered Cas12b effector protein comprises two split Cas12b effector polypeptides, each fused to a transactivation domain. In some embodiments, the engineered Cas12b effector protein further comprises one or more nuclear localization sequences (e.g., any of SEQ ID NOs: 61, 62, and 82).

In some embodiments, the engineered Cas12b effector protein is a transcriptional repressor. In some embodiments, the engineered Cas12b effector protein comprises an enzymatically inactive variant (e.g., any of SEQ ID NOs: 79-81) of any one of the engineered Cas12b nucleases described herein fused to a transcription repressor domain (e.g., KRAB). In some embodiments, the transcription repressor domain is selected from the group consisting of Kruppel associated box (KRAB), EnR, NuE, NcoR, SID, SID4X, and combinations thereof. In some embodiments, the engineered Cas12b effector protein comprises two split Cas12b effector polypeptides, each fused to a transcription repressor domain. In some embodiments, the engineered Cas12b effector protein further comprises one or more nuclear localization sequences (e.g., any of SEQ ID NOs: 61, 62, and 82).

In some embodiments, the engineered Cas12b effector protein is a base editor, such as a cytosine editor or an adenosine editor. In some embodiments, the engineered Cas12b effector protein comprises an enzymatically inactive variant (e.g., any of SEQ ID NOs: 79-81) of any one of the engineered Cas12b nucleases described herein fused to a nucleobase-editing domain, such as a cytosine base editing (CBE) domain or an adenosine base editing (ABE) domain. In some embodiments, the nucleobase-editing domain is a DNA-editing domain. In some embodiments, the nucleobase-editing domain has deaminase activity. In some embodiments, the nucleobase-editing domain is a cytosine deaminase domain. In some embodiments, the nucleobase-editing domain is an adenosine deaminase domain. Exemplary base editors based on Cas nucleases have been described, for example, in WO2018/165629A1 and WO2019/226953A1, the contents of each of which are incorporated herein by reference in their entirety. Exemplary CBE domains include, but are not limited to, activation-induced cytidine deaminase or AID (e.g., hAID), apolipoprotein B mRNA-editing complex or APOBEC (e.g., rat APOBEC1, hAPOBEC3 A/B/C/D/E/F/G) and PmCDA1. Exemplary ABE domains include, but are not limited to, TadA, ABE8 and variants thereof (see, e.g., Gaudelli et al., 2017, Nature 551: 464-471; and Richter et al., 2020, Nature Biotechnology 38: 883-891; the contents of each of which are incorporated herein by reference in their entirety). In some embodiments, the functional domain is an APOBEC1 domain, e.g., a rat APOBEC1 domain. In some embodiments, the functional domain is a TadA domain. In some embodiments, the engineered Cas12b effector protein further comprises one or more nuclear localization sequences (e.g., any of SEQ ID NOs: 61, 62, and 82).

In some embodiments, the engineered Cas12b effector protein is a prime editor. Prime editors based on Cas9 have been described, for example, in A. Anzalone et al., Nature, 2019, 576 (7785): 149-157, the content of which is incorporated herein by reference in its entirety. In some embodiments, the engineered Cas12b effector protein comprises a nickase variant of any one of the engineered Cas12b nucleases described herein fused to a reverse transcriptase domain. In some embodiments, the functional domain is a reverse transcriptase domain. In some embodiments, the reverse transcriptase domains is an M-MLV reverse transcriptase, or a variant thereof, e.g., M-MLV reverse transcriptase having one or more mutations of D200N, T306K, W313F, T330P and L603W. In some embodiments, there is provided an engineered CRISPR/Cas12b system comprising the prime editor. In some embodiments, the engineered CRISPR/Cas12b system further comprises a second Cas12b nickase, e.g., based on the same engineered Cas12b nuclease as the prime editor. In some embodiments, the engineered CRISPR/Cas12b system comprises a prime editor guide RNA (pegRNA), which comprises a primer binding site and a reverse transcriptase (RT) template sequence.

In some embodiments, the present application provides a split Cas12b effector system having one or more (e.g., 1, 2, 3, 4, 5, 6, or more) functional domains associated with (i.e., bound to or fused to) one or both split Cas12b effector portions. The functional domain(s) may be provided as part of the first and/or second split Cas12b effector proteins, as fusions within that construct. The functional domains are typically fused to other parts in the split Cas12b effector proteins (e.g., split Cas12b effector portions) via a peptide linker, such as GS linker. The functional domains can be used to repurpose the function of the split Cas12b effector system based on a catalytically dead Cas12b effector.

In some embodiments, the engineered Cas12b effector proteins comprise one or more nuclear localization sequences (NLSs) and/or one or more nuclear exportation sequences (NESs). Exemplary NLS sequences include, for example, PKKKRKV (SEQ ID NO: 82), PKKKRKVPG (SEQ ID NO: 61) and ASPKKKRKV (SEQ ID NO: 62). The NLS(s) and/or NES(s) may be operably linked to the N-terminus and/or the C-terminus of the engineered Cas12b effector proteins or polypeptide chains in the engineered Cas12b effector proteins.

In some embodiments, the engineered Cas12b effector proteins may encode additional components, such as reporter proteins. In some embodiments, the engineered Cas12b effector protein comprises a fluorescent protein, e.g., GFP. Such system could allow imaging of genomic loci (see, for example, “Dynamic Imaging of Genomic Loci in Living Human Cells by an Optimized CRISPR/Cas System” Chen B et al. Cell 2013). In some embodiments, the engineered Cas12b effector protein is an inducible split Cas effector system that can be used to image genomic loci.

### Engineered CRISPR-Cas12b Systems

Also provided are engineered CRISPR-Cas12b systems comprising: (a) any one of the engineered Cas12b nucleases or variants or derivatives thereof (e.g., any of SEQ ID NOs: 2-22 and 79-81) or the engineered Cas12b effector proteins (e.g., engineered Cas12b nuclease, nickase, split Cas12b proteins, transcriptional repressor, transcriptional activator, base editor, or prime editor) described herein, or a nucleic acid encoding thereof; and (b) a guide RNA comprising a guide sequence complementary to a target sequence of a target nucleic acid, or one or more nucleic acids encoding the guide RNA, wherein the engineered Cas12b nuclease or engineered Cas12b effector protein and the guide RNA are capable of forming a CRISPR complex that specifically binds to a target nucleic acid comprising the target sequence and induces a modification of the target nucleic acid. In some embodiments, there is provided an engineered CRISPR-Cas12b system comprising: (a) an engineered Cas12b nuclease or effector protein thereof, comprising one, two, or three types of mutations with respect to a reference Cas12b nuclease, wherein the mutations comprise: (1) substitution of one or more amino acid residues in the reference Cas12b nuclease that interact with a PAM (e.g., one or more of the following positions: 116, 123, 130, 132, 144, 145, 153, 173, 222, 395, 400, and 475) with a positively charged amino acid residue (e.g., R, H, K); and/or (2) substitution of one or more amino acid residues in the reference Cas12b nuclease that are involved in opening DNA double strands (e.g., one or more of the following positions: 118 and 119) with an amino acid residue having an aromatic ring (e.g., F, Y, W); and/or (3) substitution of one or more amino acid residues in the RuvC domain of the reference Cas12b nuclease that interact with a ssDNA substrate (e.g., one or more of the following positions: 300, 301, 304, 329, 636, 639, 647, 682, 757, 758, 761, 764, 768, 852, 854, 856, 857, 858, 860, 862, 863, 865, 866, 867, 869, 938, 956, 957, 958, 994, 1093, and 1097) with a positively charged amino acid residue (e.g., R, H, K) or a hydrophobic amino acid residue (e.g., F, Y, W, M), wherein the reference Cas12b nuclease comprises the amino acid sequence of SEQ ID NO: 1, or a nucleic acid encoding the engineered Cas12b nuclease or effector protein thereof; and (b) a gRNA comprising a guide sequence complementary to a target sequence of a target nucleic acid, or a nucleic acid encoding the gRNA, wherein the engineered Cas12b nuclease or effector protein thereof and the gRNA are capable of forming a CRISPR complex that specifically binds to a target nucleic acid comprising the target sequence and induces a modification of the target nucleic acid. In some embodiments, the engineered CRISPR-Cas12b system comprises one or more nucleic acids encoding the engineered Cas12b nuclease or variant or derivative thereof or the engineered Cas12b effector protein, and/or the guide RNA. In some embodiments, the gRNA comprises a crRNA and a tracrRNA. In some embodiments, the engineered CRISPR-Cas12b system comprises a precursor guide RNA array that can be processed, e.g., by the engineered Cas12b nuclease or variant or derivative thereof or the engineered Cas12b effector protein, into a plurality of crRNAs. In some embodiments, the gRNA is an sgRNA. In some embodiments, the sgRNA comprises a scaffold sequence of any one of SEQ ID NOs: 23-53. In some embodiments, the engineered CRISPR-Cas12b system comprises one or more vectors encoding the engineered Cas12b nuclease or variant or derivative thereof or the engineered Cas12b effector protein, and/or the guide RNA. In some embodiments, the engineered Cas12b nuclease or variant or derivative thereof or the engineered Cas12b effector protein, and/or the guide RNA are encoded by one or more vectors such as adeno-associated viral (AAV) vectors. In some embodiments, the engineered CRISPR-Cas12b system comprises a ribonucleoprotein (RNP) complex comprising the engineered Cas12b nuclease or variant or derivative thereof or the engineered Cas12b effector protein bound to the guide RNA.

In some embodiments, there is provided an engineered CRISPR-Cas12b system comprising: (a) a Cas12b nuclease comprising the amino acid sequence of SEQ ID NO: 1 or an effector protein thereof (e.g., nickase, split Cas12b proteins, transcriptional repressor, transcriptional activator, base editor, or prime editor), or any one of the engineered Cas12b nucleases or variants or derivatives thereof (e.g., any of SEQ ID NOs: 2-22 and 79-81) or the engineered Cas12b effector proteins (e.g., nickase, split Cas12b proteins, transcriptional repressor, transcriptional activator, base editor, or prime editor) described herein, or a nucleic acid encoding thereof; and (b) a gRNA comprising a guide sequence complementary to a target sequence of a target nucleic acid, or a nucleic acid encoding the gRNA, wherein the gRNA comprises an engineered scaffold comprising the sequence of any of SEQ ID NOs: 25-53; wherein i) the Cas12b nuclease or effector protein thereof or the engineered Cas12b nuclease or variant or derivative thereof or the engineered Cas12b effector protein and ii) the gRNA are capable of forming a CRISPR complex that specifically binds to the target nucleic acid and inducing a modification of the target nucleic acid. In some embodiments, there is provided an engineered CRISPR-Cas12b system comprising: (a) a Cas12b nuclease comprising the amino acid sequence of SEQ ID NO: 1 or an effector protein thereof (e.g., nickase, split Cas12b proteins, transcriptional repressor, transcriptional activator, base editor, or prime editor), or an engineered Cas12b nuclease or effector protein thereof, comprising one, two, or three types of mutations with respect to a reference Cas12b nuclease, wherein the mutations comprise: (1) substitution of one or more amino acid residues in the reference Cas12b nuclease that interact with a PAM (e.g., one or more of the following positions: 116, 123, 130, 132, 144, 145, 153, 173, 222, 395, 400, and 475) with a positively charged amino acid residue (e.g., R, H, K); and/or (2) substitution of one or more amino acid residues in the reference Cas12b nuclease that are involved in opening DNA double strands (e.g., one or more of the following positions: 118 and 119) with an amino acid residue having an aromatic ring (e.g., F, Y, W); and/or (3) substitution of one or more amino acid residues in the RuvC domain of the reference Cas12b nuclease that interact with a ssDNA substrate (e.g., one or more of the following positions: 300, 301, 304, 329, 636, 639, 647, 682, 757, 758, 761, 764, 768, 852, 854, 856, 857, 858, 860, 862, 863, 865, 866, 867, 869, 938, 956, 957, 958, 994, 1093, and 1097) with a positively charged amino acid residue (e.g., R, H, K) or a hydrophobic amino acid residue (e.g., F, Y, W, M), wherein the reference Cas12b nuclease comprises the amino acid sequence of SEQ ID NO: 1, or a nucleic acid encoding the Cas12b nuclease or effector protein thereof; and (b) a gRNA comprising a guide sequence complementary to a target sequence of a target nucleic acid, or a nucleic acid encoding the gRNA, wherein the gRNA comprises an engineered scaffold comprising the sequence of any of SEQ ID NOs: 25-53; wherein i) the Cas12b nuclease or effector protein thereof or the engineered Cas12b nuclease or effector protein thereof and ii) the gRNA are capable of forming a CRISPR complex that specifically binds to the target nucleic acid and inducing a modification of the target nucleic acid. In some embodiments, there is provided an engineered CRISPR-Cas12b system comprising: (a) a Cas12b nuclease or a Cas12b effector protein comprising the amino acid sequence of any of SEQ ID NOs: 1-22 and 79-81, or a nucleic acid encoding thereof, and (b) a gRNA comprising a guide sequence complementary to a target sequence of a target nucleic acid, or a nucleic acid encoding the gRNA, wherein the gRNA comprises an engineered scaffold comprising the sequence of any of SEQ ID NOs: 25-53; wherein the Cas12b nuclease or the Cas12b effector protein and the gRNA are capable of forming a CRISPR complex that specifically binds to the target nucleic acid and inducing a modification of the target nucleic acid. In some embodiments, the gRNA comprises a crRNA and a tracrRNA, and wherein the tracrRNA comprises the engineered scaffold or portion thereof. In some embodiments, the engineered CRISPR-Cas12b system comprises a precursor gRNA array encoding a plurality of crRNAs. In some embodiments, the gRNA is an sgRNA. In some embodiments, the engineered CRISPR-Cas12b system comprises one or more vectors encoding the engineered Cas12b nuclease, the engineered Cas12b effector protein, the Cas12b nuclease, or the Cas12b effector protein. In some embodiments, the one or more vectors are AAV vectors. In some embodiments, the one or more vectors further encode the gRNA.

### PAMs

In some embodiments, the engineered Cas12b nuclease or variant or derivative thereof, the engineered Cas12b effector protein, the Cas12b nuclease, or the Cas12b effector protein Cas12b recognizes a PAM comprising (or consisting of) the sequence of 5′-TTN-3′ (wherein N is A, T, G, or C). In some embodiments, the PAM comprises or consists of 5′-TTC-3′, 5′-TTA-3′, 5′-TTT-3′, or 5′-TTG-3′.

### Guide RNAs

The engineered CRISPR-Cas12b systems of the present application may comprise any suitable guide RNAs. A guide RNA (gRNA) may comprise a guide sequence (or spacer) capable of hybridizing to a target sequence in a target nucleic acid of interest, such as a genomic locus of interest in a cell. In some embodiments, the gRNA comprises a CRISPR RNA (crRNA) sequence comprising the guide sequence. In some embodiments, the crRNAs described herein include a direct repeat (DR) sequence and a spacer sequence. In certain embodiments, the crRNA comprises, consists essentially of, or consists of a direct repeat sequence linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence. In some embodiments, the crRNA comprises a direct repeat sequence, a spacer sequence, and a direct repeat sequence (DR-spacer-DR), which is typical of precursor crRNA (pre-crRNA) configurations. In some embodiments, the crRNA comprises a truncated direct repeat sequence and a spacer sequence, which is typical of processed or mature crRNA. In some embodiments, the crRNA comprises a mutant DR sequence and a spacer sequence. In some embodiments, the gRNA comprises a trans-activating CRISPR RNA (tracrRNA) sequence. In some embodiments, the tracrRNA is fused to the crRNA at the 5′ end of the DR sequence. In some embodiments, the guide RNA is a single-guide RNA (sgRNA). In some embodiments, the gRNA or sgRNA comprises a tracrRNA and a crRNA. In some embodiments, the sgRNA comprises the sequence of any one of SEQ ID NOs: 23-53. In some embodiments, the tracrRNA comprises the sequence of any one of SEQ ID NOs: 23-53 or a portion thereof.

In some embodiments, the gRNA comprises a non-cognate crRNA sequence and/or tracrRNA sequence that are not naturally found in the CRISPR loci of the reference Cas12b protein. For example, cognate tracrRNA and crRNA sequences of AaCas12b, AkCas12b, AmCas12b, BhCas12b, BsCas12b, Bs3Cas12b, LsCas12b and SbCas12b, as well as exemplary sgRNA sequences have been described in FIG. S4 and FIG. S8 of Teng F. et al., Cell Discovery (2019) 5: 23, the content of which are incorporated herein by reference in their entirety.

In some embodiments, the CRISPR-Cas12b system described herein comprises one or more gRNAs (e.g., crRNAs, tracrRNAs, or sgRNAs) (e.g., 1, 2, 3, 4, 5, 10, 15, or more), or nucleic acids encoding thereof. In some embodiments, the two or more gRNAs target different target sites, e.g., 2 target sites of the same target DNA or gene, or 2 target sites of 2 different target DNA or genes.

The sequences and lengths of the gRNAs described herein can be optimized. In some embodiments, the optimal length of the gRNA can be determined by identifying the processed form of the crRNA or by empirical length studies of the crRNA. In some embodiments, the gRNA comprises base modifications, such as in the gRNA scaffold region.

Complete complementarity is not required for spacers, provided that there is sufficient complementarity for the gRNA (e.g., crRNA or sgRNA) to function (i.e., directing the Cas12b nuclease (e.g., engineered) or effector protein thereof to the target site). The editing or cleavage efficiency by the Cas12b nuclease (e.g., engineered) or effector protein thereof mediated by the gRNA can be adjusted by introducing one or more mismatches (e.g., 1 or 2 mismatches between the spacer sequence and the target sequence, including the positions along the mismatches of the spacer/target sequence). Mismatches, such as double mismatches, have greater impact on cleavage efficiency when they are located more central to the spacer (i.e., not at the 3′ or 5′ end of the spacer). Thus, by choosing the position of mismatches along the spacer sequence, the editing or cleavage efficiency of the Cas12b nuclease (e.g., engineered) or effector protein thereof can be tuned. For example, if less than 100% editing or cleavage of the target sequence is desired (e.g., in a population of cells), 1 or 2 mismatches between the spacer sequence and the target sequence can be introduced into the spacer sequence.

In some embodiments, the guide sequence or spacer is designed to have at least one mismatch with the target sequence, such that a heteroduplex formed between the guide sequence and the target sequence comprises a non-pairing C in the guide sequence opposite to the target A, or a non-pairing A in the guide sequence opposite to the target C, for deamination on the target sequence (e.g., for base editing). In some embodiments, aside from this A-C or C-A mismatch, the degree of complementarity, when optimally aligned using a suitable alignment algorithm, is about or more than about 50%, 60%, 75%, 80%, 85%, 90%, 95%, 97.5%, 99%, or more.

The guide sequence may have a suitable length. In some embodiments, the length of the guide or spacer sequence is from about 10 nt to about 50 nt. In some embodiments, the length of the guide or spacer sequence is at least about 16 nucleotides, preferably about 16 to about 100 nucleotides, more preferably about 16 to about 50 nucleotides (e.g., about any of 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides). In some embodiments, the spacer is about 16 to about 27 nucleotides, such as any of about 17 to about 24 nucleotides, about 18 to about 24 nucleotides, or about 18 to about 22 nucleotides. In some embodiments, the guide sequence is between about 18 to about 35 nucleotides, including, for example, any one of 18, 19, 20, 21, 22, 23, 24, 25, 26, 27, 28, 29, 30, 31, 32, 33, 34 or 35 nucleotides.

In some embodiments, the guide or spacer sequence is at least about 60%, (e.g., at least about any of 70%, 75%, 80%, 85%, 90%, 90%, 91%, 92%, 93%, 94%, 95%, 96%, 97%, 98%, 99% or 100%) complementary to the target sequence. In some embodiments, there are at least about 15 (e.g., at least about any of 16, 17, 18, 19, 20, 21, 22, 23, 24, 25, 26, 27, 28, 29, 30, 35, 40, 45, 50 or more) base pairing between the spacer sequence and the target sequence of the target nucleic acid (e.g., DNA).

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art.

As use herein, target nucleic acid is used interchangeably with target sequence or target nucleic acid sequence to refer to a specific nucleic acid comprising a nucleic acid sequence complementary to all or part of a spacer in a crRNA or gRNA. In some examples, the target nucleic acid comprises a gene or a sequence within the gene. In some examples, the target nucleic acid comprises a non-coding region (e.g., a promoter). In some examples, the target nucleic acid is single-stranded. In some examples, the target nucleic acid is double-stranded. The target nucleic acid may be selected to target any target nucleic acid sequence, such as DNA or RNA sequence (e.g., mRNA).

The target nucleic acid should be associated with PAM, that is, short sequences recognized by the CRISPR complex. Depending on the nature of the CRISPR-Cas protein, the target sequence should be selected such that its complementary sequence (the complementary sequence of the target sequence) in the DNA duplex is upstream or downstream of PAM. In an embodiment of the invention, the complementary sequence of the target sequence is downstream or 3′ of PAM. The requirements for exact sequence and length of PAM vary depending on the Cas12b protein used.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as one or more hairpins. In general, degree of complementarity is with reference to the optimal alignment of the guide sequence and tracr sequence, along the length of the shorter of the two sequences. Optimal alignment may be determined by any suitable alignment algorithm, and may further account for secondary structures.

Any gRNA scaffold or tracrRNA or DR sequence that can mediate the binding of the Cas12b protein described herein to the corresponding gRNA (e.g., crRNA) can be used in the present invention. In some embodiments, the gRNA scaffold or tracrRNA or DR sequence comprises a stem-loop structure near the 5′ or 3′ end (immediately adjacent to the spacer sequence). “Stem-loop structure” refers to a nucleic acid having a secondary structure that includes regions of nucleotides known or predicted to form a double-strand (stem) portion and connected at one end by a linking region (loop) of substantially single-stranded nucleotides. The term “hairpin” structure is also used herein to refer to stem-loop structures. Such structures are well known in the art, and these terms are used in accordance with their commonly known meanings in the art. Stem-loop structures do not require precise base pairing. Thus, the stem may comprise one or more base mismatches. Alternatively, base pairing may be exact, i.e., not including any mismatches.

In some embodiments, the gRNA scaffold or tracrRNA or DR is a “functional variant” of a wildtype scaffold or tracrRNA or DR, such as a “functionally truncated version,” “functionally extended version,” or “functionally replacement version.” A “functional variant” of a gRNA scaffold or tracrRNA or DR is a 5′ and/or 3′ extended (functionally extended version) or truncated (functionally truncated version) variant of a reference scaffold or tracrRNA or DR (e.g., a parental DR), or comprises one or more insertions, deletions, and/or substitutions (functional replacement version) of one or more nucleotides relative to the reference scaffold or tracrRNA or DR (e.g., a parental DR), while still retaining at least about 20% (such as at least about any of 30%, 40%, 50%, 60%, 60%, 70%, 80%, 90%, 95%, or higher) functionality of the reference scaffold or tracrRNA or DR, i.e., the function to mediate the binding of the Cas12b nuclease (e.g., engineered) or effector protein thereof to the corresponding sgRNA or crRNA. gRNA scaffold or tracrRNA or DR functional variants typically retain stem-loop-like secondary structure or portions thereof available for binding of the Cas12b nuclease (e.g., engineered) or effector protein thereof. In some embodiments, the gRNA scaffold or tracrRNA or DR or functional variant thereof comprises at least two (e.g., 2, 3, 4, 5 or more) stem-loop-like secondary structures or portions thereof available for binding the Cas12b nuclease (e.g., engineered) or effector protein thereof.

In some embodiments, the DR or functional variant thereof comprises at least about 16 nucleotides (nt), such as 16, 17, 18, 19, 20, 21, 22, 23, 24, 25, 26, 27, 28, 29, 30, 31, 32, 33, 34, 35, 36, 37, 38, 39, 40 or more nucleotides. In some embodiments, the DR comprises about 20 nt to about 40 nt, such as about 20 nt to about 30 nt, about 22 nt to about 40 nt, about 23 nt to about 38 nt, about 23 nt to about 36 nt, or about 30 nt to about 40 nt. In some embodiments, the DR comprises 22 nt, 23 nt, or 24 nt. In some embodiments, the DR comprises 35 nt, 36 nt, or 37 nt. In some embodiments, the sgRNA scaffold or functional variant thereof comprises about 50 nt to about 180 nt, such as any of about 70 nt to about 140 nt, or about 90 nt to about 120 nt.

In some embodiments, the sgRNA comprises a scaffold sequence comprising a stem-loop structure (e.g., 1, 2, 3, 4, or more stemloops) near the 5′ end of the spacer sequence. In some embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In some embodiments, the stem made of the X and Y nucleotides, together with the loop will form a complete hairpin in the overall secondary structure; and, this may be advantageous and the amount of base pairs can be any amount that forms a complete hairpin. In some embodiments, any complementary X:Y base-pairing sequence (e.g., as to length) is tolerated, so long as the secondary structure of the entire guide molecule is preserved. In some embodiments, the loop that connects the stem made of X:Y basepairs can be any sequence of the same length (e.g., 4 or 5 nucleotides) or longer that does not interrupt the overall secondary structure of the guide molecule. In some embodiments, the stem comprises about 5-7 bp comprising complementary X and Y sequences, although stems of more or fewer basepairs are also contemplated. In some embodiments, non-Watson Crick basepairing is contemplated, where such pairing otherwise generally preserves the architecture of the stem-loop at that position. In some embodiments, the stem contained in the scaffold sequence comprises (e.g., consists of) 5 pairs of complementary bases that hybridize to each other, and the loop length is 6, 7, 8, or 9 nucleotides. In some embodiments, the stem can comprise at least 2, at least 3, at least 4, or at least 5 base pairs. In some embodiments, the stem-loop structure comprises a first stem nucleotide chain of 5 nucleotides in length; a second stem nucleotide chain of 5 nucleotides in length, wherein the first and the second stem nucleotide chains can hybridize to each other; and a cyclic nucleotide chain arranged between the first and second stem nucleotide chains, wherein the cyclic nucleotide chain comprises 6, 7 or 8 nucleotides.

In some embodiments, a natural hairpin or stem-loop structure of the guide molecule is extended or replaced by an extended stem-loop. It has been demonstrated in certain cases that extension of the stem can enhance the assembly of the guide molecule with the CRISPR-Cas protein (Chen et al. Cell. (2013); 155(7): 1479-1491). In some embodiments, the stem of the stemloop is extended by at least 1, 2, 3, 4, 5 or more complementary basepairs (i.e. corresponding to the addition of 2, 4, 6, 8, 10 or more nucleotides in the guide molecule). In some embodiments, these are located at the end of the stem, adjacent to the loop of the stemloop.

As used herein, the secondary structure of two or more sgRNAs or tracrRNAs are substantially identical or not substantially different means that these sgRNAs or tracrRNAs contain stems and/or loops differing by no more than 1, 2, or 3 nucleotides in length; in terms of nucleotide type (A, U, G, or C), the nucleotide sequences of these sgRNAs or tracrRNAs when compared by sequence alignment differ by no more than 1, 2, 3, 4, 5, 6, 7 or 8 nucleotides. In some embodiments, the secondary structure of two or more sgRNAs or tracrRNAs are substantially identical or not substantially different means that the sgRNAs or tracrRNAs contain stems that differ by at most one pair of complementary bases, and/or loops that differ by at most one nucleotide in length, and/or contain stems with same length but with mismatched bases.

In some embodiments, the gRNA scaffold sequence that can direct any of the engineered Cas12b effector protein of the invention to the target site comprises one or more nucleotide changes selected from the group consisting of nucleotide additions, insertions, deletions, and substitutions that do not result in substantial differences in secondary structure compared to scaffold sequence set forth in any of SEQ ID NOs: 23-53 or functionally truncated version thereof. In some embodiments, the gRNA scaffold comprises the sequence of any of SEQ ID NOs: 25-53, or a variant thereof comprising up to about 10 nt (e.g., 10, 9, 8, 7, 6, 5, 4, 3, 2, or 1 nt) difference.

In some embodiments, the guide RNA comprises a crRNA. In some embodiments, the engineered CRISPR-Cas12b system comprises a precursor guide RNA array encoding a plurality of crRNAs. In some embodiments, the Cas12b effector protein cleaves the precursor guide RNA array to produce a plurality of crRNAs. In some embodiments, the engineered CRISPR-Cas12b system comprises a precursor guide RNA array encoding a plurality of crRNA, wherein each crRNA comprises a different guide sequence. In some embodiments, the crRNA encoded by the precursor guide RNA array is associated with a tracrRNA.

### Constructs and Vectors

Also provided herein are constructs, vectors and expression systems encoding any one of the engineered Cas12b effector proteins (including engineered Cas12b nucleases) described herein. In some embodiments, the construct, vector, or expression system further comprises one or more gRNAs (e.g., sgRNAs) or crRNA arrays.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In general, a suitable vector contains an origin of replication functional in at least one organism, a promoter sequence, convenient restriction endonuclease sites, and one or more selectable markers. The term “vector” should also be construed to include non-plasmid and non-viral compounds, which facilitate transfer of nucleic acid into cells, such as, for example, polylysine compounds, liposomes, and the like.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. In some embodiments, the vector is a phage vector. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used.

In certain embodiments, the vector is an adeno-associated viruses (AAV) vector, e.g., AAV2, AAV8, or AAV9, which can be administered in a single dose containing at least 1×10⁵ particles (also referred to as particle units, pu) of adenoviruses or adeno-associated viruses. In some embodiments, the dose is at least about 1×10⁶ particles, at least about 1×10⁷ particles, at least about 1×10⁸ particles, or at least about 1×10⁹ particles of the adeno-associated viruses. The delivery methods and the doses are described, e.g., in WO 2016205764 and U.S. Pat. No. 8,454,972, the contents of each of which are incorporated herein by reference in their entirety.

In some embodiments, the vector is a recombinant adeno-associated virus (rAAV) vector. For example, in some embodiments, a modified AAV vector may be used for delivery. Modified AAV vectors can be based on one or more of several capsid types, including AAV1, AV2, AAV5, AAV6, AAV8, AAV8.2. AAV9, AAV rh10, modified AAV vectors (e.g., modified AAV2, modified AAV3, modified AAV6) and pseudotyped AAV (e.g., AAV2/8, AAV2/5 and AAV2/6). Exemplary AAV vectors and techniques that may be used to produce rAAV particles are known in the art (see, e.g., Aponte-Ubillus et al. (2018) Appl. Microbiol. Biotechnol. 102(3): 1045-54; Zhong et al. (2012) J. Genet. Syndr. Gene Ther. S1: 008; West et al. (1987) Virology 160: 38-47 (1987); Tratschin et al. (1985) Mol. Cell. Biol. 5: 3251-60); U.S. Pat. Nos. 4,797,368 and 5,173,414; and International Publication Nos. WO 2015/054653 and WO 93/24641, each of which is incorporated by reference).

Any one of the known AAV vectors for delivering Cas9 and other Cas12b proteins may be used for delivery of the engineered Cas12b nucleases or effector proteins or systems of the present application.

Methods of introducing vectors into a mammalian cell are known in the art. The vectors can be transferred into a host cell by physical, chemical, or biological methods.

Physical methods for introducing the vector into a host cell include calcium phosphate precipitation, lipofection, particle bombardment, microinjection, electroporation, and the like. Methods for producing cells comprising vectors and/or exogenous nucleic acids are well known in the art. See, for example, Sambrook et al. (2001) Molecular Cloning: A Laboratory Manual, Cold Spring Harbor Laboratory, New York. In some embodiments, the vector is introduced into the cell by electroporation.

Biological methods for introducing the heterologous nucleic acid into a host cell include the use of DNA and RNA vectors. Viral vectors have become the most widely used method of inserting genes into mammalian, e.g., human cells.

Chemical means for introducing the vector into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle). In some embodiments, the engineered CRISPR-Cas12b system is delivered as an RNP in a nanoparticle.

In some embodiments, the vector(s) or expression system encoding the CRISPR-Cas12b systems or components thereof comprise one or more selectable or detectable markers that provide a means to isolate or efficiently select cells that contain and/or have been modified by the CRISPR-Cas12b system, e.g., at an early stage and on a large scale.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)).

Other methods to confirm the presence of the heterologous nucleic acid in a host cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

In some embodiments, the nucleic acid sequences encoding the encoding the engineered Cas12b nuclease(s) or effector protein(s) and/or the guide RNA are operably linked to a promoter. In some embodiments, the promoter is an endogenous promoter with respect to a cell that is engineered using the engineered CRISPR-Cas12b system. For example, the nucleic acid encoding the engineered Cas12b effector protein may be knocked-in to the genome of an engineered mammalian cell downstream of an endogenous promoter using any methods known in the art. In some embodiments, the endogenous promoter is a promoter for an abundant protein, such as beta-actin. In some embodiments, the endogenous promoter is an inducible promoter, for example, inducible by an endogenous activation signal of an engineered mammalian cell. In some embodiments, wherein the engineered mammalian cell is a T cell, the promoter is a T cell activation-dependent promoter (such as an IL-2 promoter, an NFAT promoter, or an NFxB promoter).

In some embodiments, the promoter is a heterologous promoter with respect to a cell that is engineered using the engineered CRISPR-Cas12b system. Varieties of promoters have been explored for gene expression in mammalian cells, and any of the promoters known in the art may be used in the present application. Promoters may be roughly categorized as constitutive promoters or regulated promoters, such as inducible promoters.

In some embodiments, the nucleic acid sequences encoding the engineered Cas12b effector protein and/or the guide RNA are operably linked to a constitutive promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken j-Actin promoter coupled with CMV early enhancer (CAG). In some embodiments, the promoter is a CAG promoter comprising a cytomegalovirus (CMV) early enhancer element, the promoter, the first exon and the first intron of chicken beta-actin gene, and the splice acceptor of the rabbit beta-globin gene.

In some embodiments, the nucleic acid sequences encoding the engineered CRISPR-Cas12b protein(s) and/or the guide RNA are operably linked to an inducible promoter. Inducible promoters belong to the category of regulated promoters. The inducible promoter can be induced by one or more conditions, such as a physical condition, microenvironment, or the physiological state of a host cell, an inducer (i.e., an inducing agent), or a combination thereof. In some embodiments, the inducing condition is selected from the group consisting of: an inducer, irradiation (such as ionizing radiation, light), temperature (such as heat), redox state, tumor environment, and the activation state of a cell to be engineered by the engineered CRISPR-Cas12b system. In some embodiments, the promoter is inducible by a small molecule inducer, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most mature system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the encoding the engineered CRISPR-Cas12b protein(s) and/or the guide RNA in the present application.

In some embodiments, the nucleic acid sequence encoding the engineered Cas12b nuclease or effector protein is codon optimized. In some embodiments, the expression construct encodes a tag (e.g., a 10×His tag) operably linked to the C terminus of the engineered Cas12b nuclease or effector protein. In some embodiments, each engineered split Cas12b constructs encodes a fluorescent protein, such as GFP or RFP. The reporter proteins may be used to assess co-localization and/or dimerization of the engineered split Cas12b proteins, e.g., using microscopy. A nucleic acid sequence encoding an engineered Cas12b effector protein may be fused to a nucleic acid sequence encoding an additional component using a sequence encoding a self-cleaving peptide, such as a T2A, P2A, E2A or F2A peptide.

In some embodiments, there is provided an expression construct for mammalian cells (e.g., human cells) comprising a nucleic acid sequence encoding the engineered Cas12b nuclease or effector protein. In some embodiments, the expression construct comprises the codon-optimized sequence encoding the engineered Cas12b nuclease or effector protein inserted into a pCAG-2A-eGFP vector, such that the Cas12b protein is operably linked to eGFP. In some embodiments, a second vector is provided for expression of a guide RNA (e.g., an sgRNA, crRNA, or pre-crRNA array) in mammalian cells (e.g., human cells). In some embodiments, the sequence encoding the guide RNA is expressed in a pUC19-U6-Aa-sgRNA vector backbone.

In some embodiments, the nucleic acid(s) encoding the Cas12b protein and the nucleic acid(s) encoding the gRNA are on different vectors. In some embodiments, the nucleic acid(s) encoding the Cas12b protein and the nucleic acid(s) encoding the gRNA are on the same vector. In some embodiments, the nucleic acid(s) encoding the Cas12b protein and the nucleic acid(s) encoding the gRNA are under the control of different promoters, such as a CMV promoter and a U6 promoter. In some embodiments, the nucleic acid(s) encoding the Cas12b protein is upstream of the nucleic acid(s) encoding the gRNA. In some embodiments, the nucleic acid(s) encoding the Cas12b protein is downstream of the nucleic acid(s) encoding the gRNA. In some embodiments, the nucleic acid(s) encoding the Cas12b protein and the nucleic acid(s) encoding the gRNA are contacted with a target nucleic acid or introduced into a cell simultaneously. In some embodiments, the nucleic acid(s) encoding the Cas12b protein and the nucleic acid(s) encoding the gRNA are contacted with a target nucleic acid or introduced into a cell sequentially, such as the nucleic acid(s) encoding the Cas12b protein is introduced before the nucleic acid(s) encoding the gRNA, or the nucleic acid(s) encoding the Cas12b protein is introduced after the nucleic acid(s) encoding the gRNA. In some embodiments, the cell already expresses a Cas12b protein. In some embodiments, only the nucleic acid(s) encoding the gRNA is introduced into the cell. In some embodiments, the cell already expresses gRNA(s). In some embodiments, only the nucleic acid(s) encoding the Cas12b protein is introduced into the cell.

### III. Methods of Use

One aspect of the present application provides methods of using the any one of the engineered Cas12b nucleases or effector proteins or CRISPR-Cas12b systems described herein for detecting a target nucleic acid or modifying a nucleic acid in vitro, ex vivo, or in vivo, as well as methods of treatment or diagnosis using the engineered Cas12b nucleases or effector proteins or CRISPR-Cas12b systems. Also provided are use of the engineered Cas12b nucleases or effector proteins or CRISPR-Cas12b systems described herein for detecting or modifying a nucleic acid in a cell, and for treating or diagnosing a disease or condition in a subject; and compositions comprising any one of the engineered Cas12b nucleases or effector proteins or one or more components of the engineered CRISPR-Cas12b systems for use in the manufacture of a medicament for detecting or modifying a nucleic acid (e.g., in a cell), and for treating or diagnosing a disease or condition in a subject.

### Methods of Modification

In some embodiments, the present application provides a method of modifying a target nucleic acid comprising a target sequence, comprising contacting the target nucleic acid with any one of the engineered CRISPR-Cas12b systems described herein or components thereof. For example, when a Cas12b protein or nucleic acid encoding thereof is already present, then only the gRNA or nucleic acid encoding thereof needs to be further provided; when a gRNA or nucleic acid encoding thereof is already present, then only the Cas12b protein or nucleic acid encoding thereof needs to be further provided. In some embodiments, there is provided a method of modifying a target nucleic acid comprising a target sequence, comprising contacting (e.g., in vitro, ex vivo, or in vivo) the target nucleic acid with a CRISPR-Cas12b system (e.g., engineered, non-naturally occurring), wherein the CRISPR-Cas12b system comprises: (a) an engineered Cas12b nuclease or effector protein thereof (e.g., nickase, split Cas12b proteins, transcriptional repressor, transcriptional activator, base editor, or prime editor), comprising one, two, or three types of mutations with respect to a reference Cas12b nuclease, wherein the mutations comprise: (1) substitution of one or more amino acid residues in the reference Cas12b nuclease that interact with a PAM (e.g., one or more of the following positions: 116, 123, 130, 132, 144, 145, 153, 173, 222, 395, 400, and 475) with a positively charged amino acid residue (e.g., R, H, K); and/or (2) substitution of one or more amino acid residues in the reference Cas12b nuclease that are involved in opening DNA double strands (e.g., one or more of the following positions: 118 and 119) with an amino acid residue having an aromatic ring (e.g., F, Y, W); and/or (3) substitution of one or more amino acid residues in the RuvC domain of the reference Cas12b nuclease that interact with a ssDNA substrate (e.g., one or more of the following positions: 300, 301, 304, 329, 636, 639, 647, 682, 757, 758, 761, 764, 768, 852, 854, 856, 857, 858, 860, 862, 863, 865, 866, 867, 869, 938, 956, 957, 958, 994, 1093, and 1097) with a positively charged amino acid residue (e.g., R, H, K) or a hydrophobic amino acid residue (e.g., F, Y, W, M), wherein the reference Cas12b nuclease comprises the amino acid sequence of SEQ ID NO: 1, or a nucleic acid encoding the engineered Cas12b nuclease or effector protein thereof; and (b) a gRNA comprising a guide sequence complementary to the target sequence of the target nucleic acid, or a nucleic acid encoding the gRNA, wherein the hybridization of the guide sequence and the target sequence of the target nucleic acid mediates the contact of the engineered Cas12b nuclease or effector protein thereof with the target sequence of the target nucleic acid, resulting in the modification of the target nucleic acid by the engineered Cas12b nuclease or effector protein thereof. In some embodiments, the gRNA comprises a scaffold comprising the sequence of any of SEQ ID NOs: 23 and 25-53. In some embodiments, the engineered Cas12b nuclease or effector protein thereof comprises the amino acid sequence of any of SEQ ID NOs: 2-22 and 79-81. In some embodiments, there is provided a method of modifying a target nucleic acid comprising a target sequence, comprising contacting (e.g., in vitro, ex vivo, or in vivo) the target nucleic acid with a CRISPR-Cas12b system (e.g., engineered, non-naturally occurring), wherein the CRISPR-Cas12b system comprises: (a) a Cas12b nuclease comprising the amino acid sequence of SEQ ID NO: 1 or an effector protein thereof (e.g., nickase, split Cas12b proteins, transcriptional repressor, transcriptional activator, base editor, or prime editor), or an engineered Cas12b nuclease or effector protein thereof, comprising one, two, or three types of mutations with respect to a reference Cas12b nuclease, wherein the mutations comprise: (1) substitution of one or more amino acid residues in the reference Cas12b nuclease that interact with a PAM (e.g., one or more of the following positions: 116, 123, 130, 132, 144, 145, 153, 173, 222, 395, 400, and 475) with a positively charged amino acid residue (e.g., R, H, K); and/or (2) substitution of one or more amino acid residues in the reference Cas12b nuclease that are involved in opening DNA double strands (e.g., one or more of the following positions: 118 and 119) with an amino acid residue having an aromatic ring (e.g., F, Y, W); and/or (3) substitution of one or more amino acid residues in the RuvC domain of the reference Cas12b nuclease that interact with a ssDNA substrate (e.g., one or more of the following positions: 300, 301, 304, 329, 636, 639, 647, 682, 757, 758, 761, 764, 768, 852, 854, 856, 857, 858, 860, 862, 863, 865, 866, 867, 869, 938, 956, 957, 958, 994, 1093, and 1097) with a positively charged amino acid residue (e.g., R, H, K) or a hydrophobic amino acid residue (e.g., F, Y, W, M), wherein the reference Cas12b nuclease comprises the amino acid sequence of SEQ ID NO: 1, or a nucleic acid encoding the Cas12b nuclease (e.g., engineered) or effector protein thereof; and (b) a gRNA comprising a guide sequence complementary to the target sequence of the target nucleic acid, or a nucleic acid encoding the gRNA, wherein the gRNA comprises an engineered scaffold comprising the sequence of any of SEQ ID NOs: 25-53; wherein the hybridization of the guide sequence and the target sequence of the target nucleic acid mediates the contact of the Cas12b nuclease (e.g., engineered) or effector protein thereof with the target sequence of the target nucleic acid, resulting in the modification of the target nucleic acid by the Cas12b nuclease (e.g., engineered) or effector protein thereof. In some embodiments, the engineered Cas12b nuclease or effector protein thereof comprises the sequence of any of SEQ ID NOs: 2-22 and 79-81. In some embodiments, the method further comprises providing a repair/donor template comprising a repair/donor nucleic acid, wherein the repair/donor nucleic acid is capable of being incorporated into the modified target nucleic acid at the target sequence (e.g., via homologous recombination). In some embodiments, the modification of the target nucleic acid repairs a mutation (e.g., loss of function mutation) in the target nucleic acid to a wild-type (or non-deleterious version) sequence. In some embodiments, the modification of the target nucleic acid introduces an exogenous sequence. In some embodiments, the method is carried out in vitro. In some embodiments, the target nucleic acid is present in a cell. In some embodiments, the cell is a bacterial cell, a yeast cell, a plant cell, or an animal cell (e.g., a mammalian cell, such as human or mouse cell). In some embodiments, the method is carried out ex vivo. In some embodiments, the method is carried out in vivo. In some embodiments, the target nucleic acid is cleaved or the target sequence in the target nucleic acid is altered (e.g., base edited) by the engineered CRISPR-Cas12b system. In some embodiments, expression of the target nucleic acid is altered by the engineered CRISPR-Cas12b system. In some embodiments, the target nucleic acid is a genomic DNA, such as within a cell. In some embodiments, the target sequence is associated with a disease or condition. In some embodiments, the method of modifying of the target sequence treats the disease or condition associated with the target sequence. In some embodiments, the engineered CRISPR-Cas12b system comprises a precursor guide RNA array encoding a plurality of crRNA, wherein each crRNA comprises a different guide sequence.

In some embodiments, the present application provides a method of treating a disease or condition associated with a target nucleic acid in cells of an individual, comprising modifying the target nucleic acid in the cells of the individual using any one of the methods of modifying a target nucleic acid described herein, thereby treating the disease or condition. In some embodiments, the disease or condition is selected from the group consisting of cancer, cardiovascular diseases, hereditary diseases, autoimmune diseases, metabolic diseases, neurodegenerative diseases, ocular diseases, bacterial infections and viral infections.

The engineered CRISPR-Cas12b systems described herein can modify a target nucleic acid in a cell in a variety of ways, depending on the types of engineered Cas12b effector protein in the CRISPR-Cas12b system. In some embodiments, the method induces a site-specific cleavage in the target nucleic acid. In some embodiments, the method cleaves a genomic DNA in a cell, such as a bacterial cell, a plant cell, or an animal cell (e.g., a mammalian cell). In some embodiments, the method kills a cell by cleaving a genomic DNA in the cell. In some embodiments, the method cleaves a viral nucleic acid in a cell. In some embodiments, the method base-edits a target nucleic acid, such as repairs a deleterious or disease-related mutation to non-disease-related sequence. In some embodiments, the method enhances (e.g., increasing at least about any of 10%, 20%, 30%, 40%, 50%, 60%, 70%, 80%, 90%, 1-fold, 2-fold, 5-fold, 10-fold, 20-fold, or more) the expression of a target nucleic acid (e.g., fixing a deleterious mutation that down-regulates expression). In some embodiments, the method decreases (e.g., reducing at least about any of 10%, 20%, 30%, 40%, 50%, 60%, 70%, 80%, 90%, 1-fold, 2-fold, 5-fold, 10-fold, 20-fold, or more) the expression of a target nucleic acid (e.g., fixing a deleterious mutation that up-regulates expression).

In some embodiments, the method alters (such as increase or decrease) the expression level of the target nucleic acid in the cell. In some embodiments, the method increases the expression level of the target nucleic acid in the cell, e.g., using an engineered Cas12b effector protein based on an enzymatically inactive Cas12b protein (e.g., any of SEQ ID NOs: 79-81) fused to a transactivation domain(s). In some embodiments, the method reduces the expression level of the target nucleic acid in the cell, e.g., using an engineered Cas12b effector protein based on an enzymatically inactive Cas12b protein (e.g., any of SEQ ID NOs: 79-81) fused to a transcription repressor domain(s) (such as KRAB domain). In some embodiments, the method introduces epigenetic modifications to the target nucleic acid in the cell, e.g., using an engineered Cas12b effector protein based on an enzymatically inactive Cas12b protein (e.g., any of SEQ ID NOs: 79-81) fused to epigenetic modification domains. In some embodiments, the method introduces base-editing into the target nucleic acid in the cell, e.g., using an engineered Cas12b effector protein based on an enzymatically inactive Cas12b protein (e.g., any of SEQ ID NOs: 79-81) fused to a cytosine deaminase domain or an adenosine deaminase domain (e.g., TadA) or functional fragment thereof. The engineered Cas12b systems described herein may be used to introduce other modifications to the target nucleic acid, depending on the functional domains comprised by the engineered Cas12b effector proteins.

In some embodiments, the method alters a target sequence in the target nucleic acid in the cell. In some embodiments, the method introduces a mutation to the target nucleic acid in the cell. In some embodiments, the method uses one or more endogenous DNA repair pathways, such as Non-homologous end joining (NHEJ) or Homology directed recombination (HDR), in the cell to repair a double-strand break induced in a target DNA as a result of sequence-specific cleavage by the CRISPR complex. Exemplary mutations include, but are not limited to, insertions, deletions, substitutions, and frameshifts. In some embodiments, the method inserts a donor DNA at the target locus. In some embodiments, the insertion of the donor DNA results in introduction of a selection marker or a reporter protein to the cell. In some embodiments, the insertion of the donor DNA results in knock-in of a gene. In some embodiments, the insertion of the donor DNA results in a knockout mutation. In some embodiments, the insertion of the donor DNA results in a substitution mutation, such as a single nucleotide substitution. In some embodiments, the method induces a phenotypic change to the cell.

In some embodiments, the engineered CRISPR-Cas12b system is used a part of a genetic circuit, or for inserting a genetic circuit into the genomic DNA of a cell. The inducer-controlled engineered split Cas12b effector proteins described herein may be especially useful as a component of a genetic circuit. Genetic circuits can be useful for gene therapy. Methods and techniques of designing and using genetic circuits are known in the art. Further reference may be made to, for example, Brophy, Jennifer A N, and Christopher A. Voigt. “Principles of genetic circuit design.” Nature methods 11.5 (2014): 508.

The engineered CRISPR-Cas12b systems described herein are useful for modifying a wide range of target nucleic acids. In some embodiments, the target nucleic acid is in a cell. In some embodiments, the target nucleic acid is a genomic DNA. In some embodiments, the target nucleic acid is an extrachromosomal DNA. In some embodiments, the target nucleic acid is exogenous to a cell. In some embodiments, the target nucleic acid is a viral nucleic acid, such as viral DNA. In some embodiments, the target nucleic acid is a plasmid is a cell. In some embodiments, the target nucleic acid is a horizontally transferred plasmid. In some embodiments, the target nucleic acid is an RNA, such as mRNA.

In some embodiments, the target nucleic acid is an isolated nucleic acid, such as an isolated DNA. In some embodiments, the target nucleic acid is present in a cell-free environment. In some embodiments, the target nucleic acid is an isolated vector, such as a plasmid. In some embodiments, the target nucleic acid is an isolated linear DNA fragment.

The methods described herein are applicable for any suitable cell type. In some embodiments, the cell is a bacterium, a yeast cell, a fungal cell, an algal cell, a plant cell, or an animal cell. (e.g., a mammalian cell, such as a human cell). In some embodiments, the cell is a cell isolated from natural sources, such as a tissue biopsy. In some embodiments, the cell is a cell isolated from an in vitro cultured cell line. In some embodiments, the cell is from a primary cell line. In some embodiments, the cell is from an immortalized cell line. In some embodiments, the cell is a genetically engineered cell.

In some embodiments, the cell is an animal cell from an organism, including but not limited to, cat, dog, mouse, rat, hamster, cattle, sheep, goat, horse, donkey, pig, deer, chicken, duck, goose, rabbit, and fish.

In some embodiments, the cell is a plant cell from an organism selected from the group consisting of maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, and a biomass crop.

In some embodiments, the cell is a mammalian cell. In some embodiments, the cell is a mouse cell, such as Neuro 2A (N2a) cell. In some embodiments, the cell is a human cell. In some embodiments, the human cell is a human embryonic kidney 293T (HEK293T or 293T) cell or a HeLa cell. In some embodiments, the mammalian cell is selected from the group consisting of an immune cell, a hepatic cell, a tumor cell, a stem cell, a neuronal cell, a zygote, a muscle cell, and a skin cell.

In some embodiments, the cell is an immune cell selected from the group consisting of a cytotoxic T cell, a helper T cell, a natural killer (NK) T cell, an iNK-T cell, an NK-T like cell, a γδ T cell, a tumor-infiltrating T cell and a dendritic cell (DC)-activated T cell. In some embodiments, the method produces a modified immune cell, such as a CAR-T cell, CAR-NK cell, or a TCR-T cell.

In some embodiments, the cell is an embryonic stem (ES) cell, an induced pluripotent stem (iPS) cell, a progenitor cell of a gamete, a gamete, a zygote, or a cell in an embryo.

The methods described herein can be used to a modify a target cell in vivo, ex vivo or in vitro and, may be conducted in a manner that alters the cell such that once modified the progeny or cell line of the modified cell retains the altered phenotype. The modified cells and progeny may be part of a multi-cellular organism such as a plant or animal with ex vivo or in vivo applications, such as genome editing and gene therapy.

In some embodiments, the method of modification is carried out ex vivo. In some embodiments, the modified cell (e.g., mammalian cell) is propagated ex vivo after introduction of the engineered CRISPR-Cas12b system into the cell. In some embodiments, the modified cell is cultured to propagate for at least about any of 1 day, 2 days, 3 days, 4 days, 5 days, 6 days, 7 days, 10 days, 12 days, or 14 days. In some embodiments, the modified cell is cultured for no more than about any of 1 day, 2 days, 3 days, 4 days, 5 days, 6 days, 7 days, 10 days, 12 days, or 14 days. In some embodiments, the modified cell is further evaluated or screened to select cells with one or more desirable phenotypes or properties, or by PCR or sequencing.

In some embodiments, the target sequence is a sequence associated with a disease or condition. Exemplary diseases or conditions include, but are not limited to, cancer, blood diseases, cardiovascular diseases, hereditary diseases, autoimmune diseases, metabolic diseases, neurological diseases, neurodegenerative diseases, ocular diseases, bacterial infections and viral infections. In some embodiments, the disease or condition is graft-versus-host disease (GvHD) or host-versus-graft (HvG) disease. In some embodiments, the disease or condition is a genetic disease. In some embodiments, the disease or condition is a monogenetic disease or condition. In some embodiments, the disease or condition is a polygenetic disease or condition.

In some embodiments, the target sequence has a mutation compared to a wild type sequence. In some embodiments, the target sequence has a single-nucleotide polymorphism (SNP) associated with a disease or condition.

In some embodiments, the donor DNA that is inserted into the target nucleic acid encodes a biological product selected from the group consisting of a reporter protein, an antigen-specific receptor, a therapeutic protein, an antibiotic resistance protein, an RNAi molecule, a cytokine, a kinase, an antigen, an antigen-specific receptor, a chimeric receptor, a cytokine receptor, and a suicide polypeptide. In some embodiments, the donor DNA encodes a therapeutic protein, e.g., cytokine. In some embodiments, the donor DNA encodes a therapeutic protein useful for gene therapy. In some embodiments, the donor DNA encodes a therapeutic antibody. In some embodiments, the donor DNA encodes an engineered receptor, such as a chimeric antigen receptor (CAR), or an engineered TCR. In some embodiments, the donor DNA encodes a therapeutic RNA, such as a small RNA (e.g., siRNA, shRNA, or miRNA), or a long non-coding RNA (lincRNA).

The methods described herein may be used for multiplex gene editing or regulation at two or more (e.g., 2, 3, 4, 5, 6, 8, 10 or more) different target loci. In some embodiments, the method detects or modifies a plurality of target nucleic acids or target nucleic acid sequences. In some embodiments, the method comprises contacting the target nucleic acid with a guide RNA comprises a plurality (e.g., 2, 3, 4, 5, 6, 8, 10 or more) of crRNA sequences, wherein each crRNA comprises a different target sequence.

Also provided are engineered cells comprising a modified target nucleic acid, which are produced using any one of the methods of modification described herein. The engineered cells may be used for cell therapy. Autologous or allogeneic cells may be used to prepare engineered cells using the methods described herein for cell therapy.

The methods described herein may also be used to generate isogenic lines of cells (e.g., mammalian cells) to study genetic variants.

Also provided are engineered plants or non-human animals comprising the engineered cells described herein. In some embodiments, the engineered plants or non-human animals are genome-edited plants or non-human animals. The engineered non-human animals can be used as disease models.

Techniques for producing non-human genome-edited or transgenic animals are well known in the art and include, but are not limited to, pronuclear microinjection, viral infection, and transformation of embryonic stem cells and induced pluripotent stem (iPS) cells. Detailed methods that can be used include, but are not limited to, those described in Sundberg and Ichiki (2006, Genetically Engineered Mice Handbook, CRC Press) and Gibson (2004, A Primer Of Genome Science 2nd ed. Sunderland, Mass.: Sinauer).

The engineered animals may be of any suitable species, including, but not limited to, such as bovids, equids, ovids, canids, cervids, felids, goats, swine, primates as well as less commonly known mammals such as elephants, deer, zebra, or camels.

### Methods of Treatment

Further provided are methods of treatment using any one of the methods of modifying a target nucleic acid in a cell described herein, and methods of diagnosis using any one of the methods of detecting a target nucleic acid described herein.

In some embodiments, the present application provides a method of treating a disease or condition associated with a target nucleic acid in cells of an individual, comprising contacting the target nucleic acid with any one of the engineered CRISPR-Cas12b systems described herein, wherein the guide sequence of the guide RNA is complementary to a target sequence of the target nucleic acid, wherein the Cas12b nuclease (e.g., engineered) or effector protein thereof (e.g., comprising any of SEQ ID NOs: 1-22 and 79-81) and the guide RNA associate with each other to bind to the target nucleic acid to modify the target nucleic acid, thereby the disease or condition is treated. In some embodiments, a mutation (e.g., knockout or knock-in mutation) is introduced to the target nucleic acid. In some embodiments, expression of the target nucleic acid is enhanced. In some embodiments, expression of the target nucleic acid is inhibited.

In some embodiments, the present application provides a method of treating a disease or condition in an individual, comprising administering to the individual an effective amount of any one of the engineered CRISPR-Cas12b systems described herein, and a donor DNA encoding a therapeutic agent, wherein the guide sequence of the guide RNA is complementary to a target sequence of a target nucleic acid of the individual, wherein the Cas12b nuclease (e.g., engineered) or effector protein thereof (e.g., comprising any of SEQ ID NOs: 1-22 and 79-81) and the guide RNA associate with each other to bind to the target nucleic acid and inserts the donor DNA in the target sequence, thereby the disease or condition is treated.

In some embodiments, the present application provides a method of treating a disease or condition in an individual, comprising administering to the individual an effective amount of engineered cells comprising a modified target nucleic acid, wherein the engineered cells are prepared by contacting the cell with any one of the engineered CRISPR-Cas12b systems described herein, wherein the guide sequence of the guide RNA is complementary to a target sequence of the target nucleic acid, wherein the Cas12b nuclease (e.g., engineered) or effector protein thereof (e.g., comprising any of SEQ ID NOs: 1-22 and 79-81) and the guide RNA associate with each other to bind to the target nucleic acid to modify the target nucleic acid. In some embodiments, the engineered cells are immune cells.

In some embodiments, there is provided a method of treating a disease or condition associated with a target nucleic acid in cells of an individual (e.g., human), comprising contacting the target nucleic acid (e.g., ex vivo, or in vivo) with or administering to the individual an effective amount of a CRISPR-Cas12b system, (e.g., engineered, non-naturally occurring), wherein the CRISPR-Cas12b system comprises: (a) an engineered Cas12b nuclease or effector protein thereof (e.g., nickase, split Cas12b proteins, transcriptional repressor, transcriptional activator, base editor, or prime editor), comprising one, two, or three types of mutations with respect to a reference Cas12b nuclease, wherein the mutations comprise: (1) substitution of one or more amino acid residues in the reference Cas12b nuclease that interact with a PAM (e.g., one or more of the following positions: 116, 123, 130, 132, 144, 145, 153, 173, 222, 395, 400, and 475) with a positively charged amino acid residue (e.g., R, H, K); and/or (2) substitution of one or more amino acid residues in the reference Cas12b nuclease that are involved in opening DNA double strands (e.g., one or more of the following positions: 118 and 119) with an amino acid residue having an aromatic ring (e.g., F, Y, W); and/or (3) substitution of one or more amino acid residues in the RuvC domain of the reference Cas12b nuclease that interact with a ssDNA substrate (e.g., one or more of the following positions: 300, 301, 304, 329, 636, 639, 647, 682, 757, 758, 761, 764, 768, 852, 854, 856, 857, 858, 860, 862, 863, 865, 866, 867, 869, 938, 956, 957, 958, 994, 1093, and 1097) with a positively charged amino acid residue (e.g., R, H, K) or a hydrophobic amino acid residue (e.g., F, Y, W, M), wherein the reference Cas12b nuclease comprises the amino acid sequence of SEQ ID NO: 1, or a nucleic acid encoding the engineered Cas12b nuclease or effector protein thereof; and (b) a gRNA comprising a guide sequence complementary to the target sequence of the target nucleic acid, or a nucleic acid encoding the gRNA, wherein the hybridization of the guide sequence and the target sequence of the target nucleic acid mediates the contact of the engineered Cas12b nuclease or effector protein thereof with the target sequence of the target nucleic acid, resulting in the modification of the target nucleic acid by the engineered Cas12b nuclease or effector protein thereof, thereby the disease or condition is treated. In some embodiments, the gRNA comprises a scaffold comprising the sequence of any of SEQ ID NOs: 23 and 25-53. In some embodiments, there is provided a method of treating a disease or condition associated with a target nucleic acid in cells of an individual (e.g., human), comprising contacting the target nucleic acid (e.g., ex vivo, or in vivo) with or administering to the individual an effective amount of a CRISPR-Cas12b system, (e.g., engineered, non-naturally occurring), wherein the CRISPR-Cas12b system comprises: (a) a Cas12b nuclease comprising the amino acid sequence of SEQ ID NO: 1 or an effector protein thereof (e.g., nickase, split Cas12b proteins, transcriptional repressor, transcriptional activator, base editor, or prime editor), or an engineered Cas12b nuclease or effector protein thereof, comprising one, two, or three types of mutations with respect to a reference Cas12b nuclease, wherein the mutations comprise: (1) substitution of one or more amino acid residues in the reference Cas12b nuclease that interact with a PAM (e.g., one or more of the following positions: 116, 123, 130, 132, 144, 145, 153, 173, 222, 395, 400, and 475) with a positively charged amino acid residue (e.g., R, H, K); and/or (2) substitution of one or more amino acid residues in the reference Cas12b nuclease that are involved in opening DNA double strands (e.g., one or more of the following positions: 118 and 119) with an amino acid residue having an aromatic ring (e.g., F, Y, W); and/or (3) substitution of one or more amino acid residues in the RuvC domain of the reference Cas12b nuclease that interact with a ssDNA substrate (e.g., one or more of the following positions: 300, 301, 304, 329, 636, 639, 647, 682, 757, 758, 761, 764, 768, 852, 854, 856, 857, 858, 860, 862, 863, 865, 866, 867, 869, 938, 956, 957, 958, 994, 1093, and 1097) with a positively charged amino acid residue (e.g., R, H, K) or a hydrophobic amino acid residue (e.g., F, Y, W, M), wherein the reference Cas12b nuclease comprises the amino acid sequence of SEQ ID NO: 1, or a nucleic acid encoding the Cas12b nuclease (e.g., engineered) or effector protein thereof; and (b) a gRNA comprising a guide sequence complementary to the target sequence of the target nucleic acid, or a nucleic acid encoding the gRNA, wherein the gRNA comprises an engineered scaffold comprising the sequence of any of SEQ ID NOs: 25-53; wherein the hybridization of the guide sequence and the target sequence of the target nucleic acid mediates the contact of the Cas12b nuclease (e.g., engineered) or effector protein thereof with the target sequence of the target nucleic acid, resulting in the modification of the target nucleic acid by the Cas12b nuclease (e.g., engineered) or effector protein thereof, thereby treating the disease or condition. In some embodiments, the engineered Cas12b nuclease or effector protein thereof comprises the amino acid sequence of any of SEQ ID NOs: 2-22 and 79-81. In some embodiments, the method further comprises contacting the target nucleic acid (e.g., ex vivo, or in vivo) with or administering to the individual an effective amount of a repair/donor nucleic acid, wherein the repair/donor nucleic acid is capable of being incorporated into the modified target nucleic acid at the target sequence (e.g., via homologous recombination). In some embodiments, the modification of the target nucleic acid repairs a mutation (e.g., loss of function mutation) in the target nucleic acid to a wild-type (or non-deleterious version) sequence. In some embodiments, the modification of the target nucleic acid introduces an exogenous sequence.

In some embodiments, the individual is a human being. In some embodiments, the individual is an animal, e.g., a model animal such as a rodent (e.g., mouse, rat, hamster), a pet (e.g., cat, dog, rabbit), or a farm animal (e.g., horse, cow, sheep, goat, donkey, pig). In some embodiments, the individual is a mammal.

In some embodiments, the disease or condition is associated with an abnormality (e.g., pathogenic point mutation) in a target nucleic acid of an individual (e.g., human). In some embodiments, the disease or condition is treated due to modification (e.g., cleavage, base editing, or repair) of the target nucleic acid (e.g., fix the abnormality) by the CRISPR-Cas12b system or complex. In some embodiments, the disease is caused by over-expression or mis-expression (e.g., missense mutation, frameshift mutation, nonsense mutation) of one or more target gene, wherein the CRISPR-Cas12b systems or complexes can target the one or more target genes for targeted modification, such as cleavage, based editing, or sequence repair (e.g., by further introducing a repair/donor template for repairing the cleaved target gene by the CRISPR-Cas12b systems or complexes by homologous recombination).

In some embodiments, the disease or condition is selected from the group consisting of cancer, cardiovascular diseases, hereditary diseases, autoimmune diseases, metabolic diseases, neurodegenerative diseases, ocular diseases, bacterial infections and viral infections.

In some embodiments, the disease or condition is selected from the group consisting of transthyretin amyloidosis (ATTR) (such as transthyretin-related wild-type amyloidosis (ATTRwt), transthyretin-related hereditary amyloidosis (ATTRm), familial amyloid polyneuropathy (FAP, ATTR-PN), or familial amyloid cardiomyopathy (FAC, ATTR-CM)), cystic fibrosis, hereditary angioedema (HAE), diabetes, progressive pseudohypertrophic muscular dystrophy, Becker muscular dystrophy (BMD), alpha-1 antitrypsin deficiency (AAT deficiency), Pompe disease, myotonic dystrophy, Huntington's disease, Fragile X syndrome (FXS), Friedreich ataxia (FRDA), amyotrophic lateral sclerosis (ALS), frontotemporal dementia (FTD), hereditary chronic kidney disease, hyperlipidemia, hypercholesterolemia (e.g., familial hypercholesterolemia), Leber congenital amaurosis (LCA), sickle cell disease (SCD), and β-thalassemia. In some embodiments, the CRISPR-Cas12b system or complex is packaged and delivered via a lipid nanoparticle. In some embodiments, the lipid nanoparticle is administered via intravenous injection or infusion to the individual.

In some embodiments, the target nucleic acid is PCSK9. In some embodiments, the disease or condition is a cardiovascular disease. In some embodiments, the disease or condition is a coronary artery disease. In some embodiments, the method reduces cholesterol levels in an individual. In some embodiments, the method treats diabetes in the individual. In some embodiments, the disease or condition is hypercholesterolemia, such as familial hypercholesterolemia.

In some embodiments, the target nucleic acid is HBG1 and/or HBG2. In some embodiments, the disease or condition is sickle cell disease or β-thalassemia. In some embodiments, the disease or condition is hereditary persistence of fetal hemoglobin (HPFH), HbS-Gene Deletion HPFH, or HbS-HPFH due to point mutations.

In some embodiments, the target nucleic acid is C-C chemokine receptor (CCR) 5 (CCR5), which encodes the main HIV-1 coreceptor. In some embodiments, the disease or condition is an infectious disease, e.g., AIDS. In some embodiments, the disease or condition is a non-infectious disease, such as cancer (e.g., breast or prostate cancer), atherosclerosis, stroke, or inflammatory bowel disease (IBD).

In some embodiments, the target nucleic acid is CD34. In some embodiments, the disease or condition is cancer.

In some embodiments, the target nucleic acid is Ring Finger Protein 2 (RNF2). In some embodiments, the disease or condition is a neurological disorder, such as Luo-Schoch-Yamamoto Syndrome or Non-Specific Syndromic Intellectual Disability.

### Methods of Detection

The present application also provides methods of using any one of the engineered Cas12b nucleases or effector proteins thereof (e.g., comprising any of SEQ ID NOs: 2-22 and 79-81) with improved activity or CRISPR-Cas12b systems for detection of a target nucleic acid. The use of Cas12b effector proteins as detection agents takes advantage of the discovery that type V CRISPR/Cas12 proteins (e.g., Cas12a, Cas12b, Cas12c, Cas12d, Cas12e (CasX), and Cas12i) can promiscuously cleave non-targeted single stranded DNA (ssDNA) once activated by detection of a target DNA. Methods of using Cas12b proteins as detection agents have been described, for example, in U.S. Ser. No. 10/253,365 and WO2020/056924, the contents of each of which are herein incorporated by reference in their entirety. In some embodiments, the detection of the target nucleic acid in a sample diagnose a disease a condition.

In some embodiments, once a Cas12b effector protein is activated by a guide RNA, which occurs when a sample includes a target DNA to which the guide RNA hybridizes (i.e., the sample includes the targeted DNA), the Cas12b nuclease or effector protein thereof becomes a nuclease that promiscuously cleaves single strand nucleic acids (e.g., non-target ssDNAs or RNAs, i.e., single strand nucleic acid to which the guide sequence of the guide RNA does not hybridize). Thus, when the targeted DNA (double or single stranded) is present in the sample (e.g., in some cases above a threshold amount), the result is cleavage of single strand nucleic acids in the sample, which can be detected using any convenient detection method (e.g., using a labeled single stranded detector nucleic acid, such as DNA or RNA). Cas12b can cleave ssDNA and ssRNA.

In some embodiments, there is provided a method of detecting a target DNA (e.g., double stranded or single stranded) in a sample, comprising: (a) contacting the sample with: (i) any one of the engineered Cas12b nucleases or effector proteins thereof described herein (e.g., comprising any of SEQ ID NOs: 2-22 and 79-81); (ii) a guide RNA comprising a guide sequence that hybridizes with the target DNA; and (iii) a detector nucleic acid that is single stranded (i.e., a “single stranded detector nucleic acid”) and does not hybridize with the guide sequence of the guide RNA; and (b) measuring a detectable signal produced by cleavage of the single stranded detector nucleic acid by the engineered Cas12b effector protein. In some embodiments, there is provided a method of detecting a target DNA (e.g., double stranded or single stranded) in a sample, comprising: (a) contacting the sample with: (i) any of the Cas12b nucleases (e.g., engineered or wildtype) or effector proteins thereof described herein (e.g., comprising any of SEQ ID NOs: 1-22 and 79-81); (ii) a guide RNA comprising a guide sequence that hybridizes with the target DNA, and engineered scaffold comprising the sequence of any of SEQ ID NOs: 25-53; and (iii) a detector nucleic acid that is single stranded (i.e., a “single stranded detector nucleic acid”) and does not hybridize with the guide sequence of the guide RNA; and (b) measuring a detectable signal produced by cleavage of the single stranded detector nucleic acid by the engineered Cas12b effector protein. In some embodiments, there is provided a method of detecting a target nucleic acid in a sample, comprising: (a) contacting the sample with any of the engineered CRISPR-Cas12b systems described herein and a labeled detector nucleic acid, wherein the gRNA comprises a guide sequence complementary to a target sequence of the target nucleic acid, and wherein the labeled detector nucleic acid is single-stranded and does not hybridize with the guide sequence of the gRNA; and (b) measuring a detectable signal produced by cleavage of the labeled detector nucleic acid by the engineered CRISPR-Cas12b system, thereby detecting the target nucleic acid. In some cases, the single stranded detector nucleic acid includes a fluorescence-emitting dye pair (e.g., a fluorescence-emitting dye pair is a fluorescence resonance energy transfer (FRET) pair, a quencher/fluor pair). In some cases, the target DNA is a viral DNA (e.g., papovavirus, hepadnavirus, herpesvirus, adenovirus, poxvirus, parvovirus, and the like). In some embodiments, the single stranded detector nucleic acid is a DNA. In some embodiments, the single stranded detector nucleic acid is an RNA. In some embodiments, the engineered Cas12b effector protein is an engineered Cas12b nuclease. In some embodiments, the method is carried out in vitro. In some embodiments, the target nucleic acid is present in a cell, such as a bacterial cell, a yeast cell, a plant cell, or an animal cell. In some embodiments, the method is carried out ex vivo. In some embodiments, the method is carried out in vivo. In some embodiments, the target nucleic acid is a genomic DNA. In some embodiments, the target sequence is associated with a disease or a condition.

A method of the present disclosure for detecting a target DNA (single-stranded or double-stranded) in a sample can detect a target DNA with a high degree of sensitivity. In some cases, a method of the present disclosure can be used to detect a target DNA present in a sample comprising a plurality of DNAs (including the target DNA and a plurality of non-target DNAs), where the target DNA is present at one or more copies per 10⁷ non-target DNAs (e.g., one or more copies per 10⁶ non-target DNAs, one or more copies per 10⁵ non-target DNAs, one or more copies per 10⁴ non-target DNAs, one or more copies per 10³ non-target DNAs, one or more copies per 10² non-target DNAs, one or more copies per 50 non-target DNAs, one or more copies per 20 non-target DNAs, one or more copies per 10 non-target DNAs, or one or more copies per 5 non-target DNAs).

In some embodiments, the engineered Cas12b nucleases or effector proteins thereof described herein (e.g., comprising any of SEQ ID NOs: 2-22) can detect a target DNA with a higher degree of sensitivity compared to the reference Cas12b nuclease (e.g., SEQ ID NO: 1). In some embodiments, the engineered Cas12b effector protein can detect a target DNA with 10%, 15%, 20%, 25%, 30%, 40%, 50%, 60%, 70%, 80%, 90% or higher sensitivity compared to the reference Cas12b nuclease.

### Methods of Delivery

In some embodiments, the engineered CRISPR-Cas12b systems described herein, or components thereof, nucleic acid molecules thereof, or nucleic acid molecules encoding or providing components thereof, can be delivered to host cells by various delivery systems such as plasmid or viral vectors (e.g., any one of the vectors described in the “Constructs and Vectors” subsection above). In some embodiments or methods, the engineered CRISPR-Cas12b systems can be delivered by other methods, such as nucleofection or electroporation of ribonucleoprotein complexes consisting of the engineered Cas12b nucleases or effector proteins thereof and their cognate RNA guide or guides.

In some embodiments, the delivery is via nanoparticles or exosomes.

In some embodiments, paired Cas12b nickase complexes can be delivered directly using nanoparticle or other direct protein delivery methods, such that complexes containing both paired crRNA elements are co-delivered. Furthermore, protein can be delivered to cells by viral vector or directly, followed by the direct delivery of a CRISPR array containing two paired spacers for double nicking. In some instances, for direct RNA delivery, the RNA may be conjugated to at least one sugar moiety, such as N-acetyl galactosamine (GalNAc) (particularly, triantennary GalNAc). In some embodiments, the CRISPR-Cas12b system or component thereof is packaged and delivered via a lipid nanoparticle. In some embodiments, the lipid nanoparticle is administered via intravenous injection or infusion to the individual.

### IV. Kits and Articles of Manufacture

Further provided are compositions, kits, unit dosages, and articles of manufacture comprising one or more components of any one of the engineered Cas12b nucleases or effector proteins thereof, sgRNAs comprising engineered scaffold (e.g., any one of SEQ ID NOs: 25-53), or engineered CRISPR-Cas12b systems described herein.

In some embodiments, there is provided a kit comprising: one or more AAV vectors encoding any one of the engineered Cas12b nucleases or effector proteins thereof, or engineered CRISPR-Cas12b systems described herein. In some embodiments, the kit further comprises one or more guide RNAs, such as sgRNAs comprising engineered scaffold (e.g., any one of SEQ ID NOs: 25-53). In some embodiments, the kit further comprises a donor DNA. In some embodiments, the kit further comprises a cell, such as a human cell.

The kits may contain one or more additional components, such as containers, reagents, culturing media, cytokines, buffers, antibodies, and the like to allow propagation of an engineered cell. The kits may also contain a device for administration of the composition.

The kit may further comprise instructions for using the engineered CRISPR-Cas12b system described herein, such as methods of detecting or modifying a target nucleic acid. In some embodiments, the kit comprises instructions for treating or diagnosing a disease or condition. The instructions relating to the use of the components of the kit generally include information as to dosage, dosing schedule, and route of administration for the intended treatment. The containers may be unit doses, bulk packages (e.g., multi-dose packages) or sub-unit doses. For example, kits may be provided that contain sufficient dosages of the composition as disclosed herein to provide effective treatment of an individual for an extended period. Kits may also include multiple unit doses of the composition and instructions for use, packaged in quantities sufficient for storage and use in pharmacies, for example, hospital pharmacies and compounding pharmacies.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual.

Package insert refers to instructions customarily included in commercial packages of therapeutic products that contain information about the indications, usage, dosage, administration, contraindications and/or warnings concerning the use of such therapeutic products.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXEMPLARY EMBODIMENTS

Embodiment 1. An engineered Cas12b nuclease, comprising one, two, or three types of mutations with respect to a reference Cas12b nuclease, wherein the mutations comprise: (1) substitution of one or more amino acid residues in the reference Cas12b nuclease that interact with a protospacer adjacent motif (PAM) with a positively charged amino acid residue; and/or (2) substitution of one or more amino acid residues in the reference Cas12b nuclease that are involved in opening DNA double strands with an amino acid residue having an aromatic ring; and/or (3) substitution of one or more amino acid residues in the RuvC domain of the reference Cas12b nuclease that interact with a single-stranded DNA substrate with a positively charged amino acid residue or a hydrophobic amino acid residue.

Embodiment 2. The engineered Cas12b nuclease of embodiment 1, wherein the reference Cas12b nuclease is a wild-type Cas12b nuclease.

Embodiment 3. The engineered Cas12b nuclease of embodiment 1 or 2, wherein the reference Cas12b nuclease comprises the amino acid sequence of SEQ ID NO: 1.

Embodiment 4. The engineered Cas12b nuclease of any one of embodiments 1-3, comprising substitution of one or more amino acid residues in the reference Cas12b nuclease that interact with PAM with a positively charged amino acid residue.

Embodiment 5. The engineered Cas12b nuclease of embodiment 4, wherein the one or more amino acid residues that interact with PAM are within 9 angstroms from PAM in a three-dimensional structure.

Embodiment 6. The engineered Cas12b nuclease of embodiment 4 or 5, wherein the one or more amino acid residues that interact with PAM are in one or more of the following positions: 116, 123, 130, 132, 144, 145, 153, 173, 222, 395, 400, and/or 475; wherein the amino acid residue numbering is according to SEQ ID NO: 1.

Embodiment 7. The engineered Cas12b nuclease of embodiment 6, wherein the one or more amino acid residues that interact with PAM comprise one or more of the following amino acid residues: D116, K123, D130, D132, N144, K145, E153, D173, Q222, D395, N400, and/or E475; wherein the amino acid residue numbering is according to SEQ ID NO: 1.

Embodiment 8. The engineered Cas12b nuclease of embodiment 7, wherein the one or more amino acid residues that interact with PAM comprise one or more of the following amino acid residues: D116 and/or E475; wherein the amino acid residue numbering is according to SEQ ID NO: 1.

Embodiment 9. The engineered Cas12b nuclease of any one of embodiments 4-8, wherein the positively charged amino acid residue is R or K.

Embodiment 10. The engineered Cas12b nuclease of embodiment 9, wherein the substitution of one or more amino acid residues in the reference Cas12b nuclease that interact with PAM with the positively charged amino acid residue are one or more of the following substitutions: D116R and/or E475R; wherein the amino acid residue numbering is according to SEQ ID NO: 1.

Embodiment 11. The engineered Cas12b nuclease of any one of embodiments 1-10, comprising substitution of one or more amino acid residues in the reference Cas12b nuclease that are involved in opening the DNA double strands with an amino acid residue having an aromatic ring.

Embodiment 12. The engineered Cas12b nuclease of embodiment 11, wherein the one or more amino acid residues that are involved in opening the DNA double strands interact with the last base pair in PAM relative to the 3′end of a target strand.

Embodiment 13. The engineered Cas12b nuclease of embodiment 11 or 12, wherein the one or more amino acid residues that are involved in opening the DNA double strands are in one or more of the following positions: 118, and/or 119; wherein the amino acid residue numbering is according to SEQ ID NO: 1.

Embodiment 14. The engineered Cas12b nuclease of any one of embodiments 11-13, wherein the amino acid residue having an aromatic ring is Y, F, or W.

Embodiment 15. The engineered Cas12b nuclease of embodiment 14, wherein the substitution of one or more amino acid residues in the reference Cas12b nuclease that are involved in opening the DNA double strands with the amino acid residue having an aromatic ring is Q119Y, Q119F, or Q119W; wherein the amino acid residue numbering is according to SEQ ID NO: 1.

Embodiment 16. The engineered Cas12b nuclease of any one of embodiments 1-16, comprising substitution of one or more amino acid residues in the reference Cas12b nuclease that are in the RuvC domain and interact with the single-stranded DNA substrate with a positively charged amino acid residue or a hydrophobic amino acid residue.

Embodiment 17. The engineered Cas12b nuclease of embodiment 16, wherein the one or more amino acid residues that are in the RuvC domain and interact with the single-stranded DNA substrate are within 9 angstroms from the single-stranded DNA substrate in a three-dimensional structure.

Embodiment 18. The engineered Cas12b nuclease of any embodiment 17, wherein the one or more amino acid residues that are in the RuvC domain and interact with the single-stranded DNA substrate are in one or more of the following positions: 300, 301, 304, 329, 636, 639, 647, 682, 757, 758, 761, 764, 768, 852, 854, 856, 857, 858, 860, 862, 863, 865, 866, 867, 869, 938, 956, 957, 958, 994, 1093, and/or 1097; wherein the amino acid residue numbering is according to SEQ ID NO: 1.

Embodiment 19. The engineered Cas12b nuclease of embodiment 18, wherein the one or more amino acid residues that are in the RuvC domain and interact with the single-stranded DNA substrate comprise one or more of the following amino acid residues: D300, K301, E304, N329, E636, Q639, T647, Q682, I757, E758, E761, E764, K768, E852, Q854, N856, N857, D858, P860, S862, E863, N865, Q866, L867, Q869, E938, E956, G957, E958, I994, Q1093, and/or W1097; wherein the amino acid residue numbering is according to SEQ ID NO: 1.

Embodiment 20. The engineered Cas12b nuclease of embodiment 19, comprising substitution of one or more of the following amino acid residues with a positively charged amino acid residue: E636, I757, E758, E761, Q854, N857, N865, Q866, Q869, and/or Q1093; wherein the amino acid residue numbering is according to SEQ ID NO: 1.

Embodiment 21. The engineered Cas12b nuclease of embodiment 20, wherein the positively charged amino acid residue is R or K.

Embodiment 22. The engineered Cas12b nuclease of embodiment 21, wherein the substitution of one or more amino acid residues in the reference Cas12b nuclease that are in the RuvC domain and interact with the single-stranded DNA substrate are one or more of the following substitutions: E636R, I757R, E758R, E761R, Q854R and/or N857K; wherein the amino acid residue numbering is according to SEQ ID NO: 1.

Embodiment 23. The engineered Cas12b nuclease of embodiment 19, comprising substitution of one or more of the following amino acid residues with a hydrophobic amino acid residue: E758, E761, E863, N865, Q866, Q869, Q956, and/or Q1093; wherein the amino acid residue numbering is according to SEQ ID NO: 1.

Embodiment 24. The engineered Cas12b nuclease of embodiment 23, wherein the hydrophobic amino acid residue is W, Y, F, or M.

Embodiment 25. The engineered Cas12b nuclease of embodiment 24, wherein the substitution of one or more amino acid residues in the reference Cas12b nuclease that are in the RuvC domain and interact with the single-stranded DNA substrate are one or more of the following substitutions: N865W, N865Y, Q866M, Q869M, Q1093W, and/or Q1093Y; wherein the amino acid residue numbering is according to SEQ ID NO: 1.

Embodiment 26. The engineered Cas12b nuclease of any one of embodiments 1-3, wherein the engineered Cas12b nuclease comprises any one of the following substitutions or combinations thereof: (1) D116R; (2) E475R; (3) Q119F and E475R; (4) Q119F, E475R, and E758R; (5) Q119Y; (6) Q119F; (7) Q119W; (8) I757R; (9) E758R; (10) E761R; (11) K768R; (12) I757R and E758R; (13) I757R and E761R; (14) I757R and K768R; (15) E758R and E761R; (16) E758R and K768R; (17) E761R and K768R; (18) I757R, E758R, and E761R; (19) I757R, E758R, and K768R; (20) I757R, E761R and K768R; (21) E758R, E761R, and K768R; (22) I757R, E758R, E761R, and K768R; (23) Q866M; (24) Q869M; and (25) Q866M and Q869M; wherein the amino acid residue numbering is according to SEQ ID NO: 1.

Embodiment 27. The engineered Cas12b nuclease of any one of embodiments 1-26, comprising an amino acid sequence having at least 85% sequence identity to any one of SEQ ID NOs: 2-22.

Embodiment 28. The engineered Cas12b nuclease of any of embodiments 1-27, further comprising one or more mutations that increase flexibility of a flexible region comprising amino acid residues 855-859; wherein the amino acid position numbers are in reference to SEQ ID NO: 1.

Embodiment 29. The engineered Cas12b nuclease of embodiment 28, wherein the one or more mutations that increase flexibility comprises N856G.

Embodiment 30. An engineered Cas12b nuclease comprising any one or more of the following mutations: (1) D116R; (2) E475R; (3) Q119F and E475R; (4) Q119F, E475R, and E758R; (5) Q119Y; (6) Q119F; (7) Q119W; (8) Q119F and E475R; (9) Q119F, E475R and E758R (10) E636R; (11) I757R; (12) E758R; (13) E761R; (14) Q854R; (15) N857K; (16) Q119F, E475R, and E758R; (17) K768R; (18) 1757R and E758R; (19) 1757R and E761R; (20) I757R and K768R; (21) E758R and E761R; (22) E758R and K768R; (23) E761R and K768R; (24) I757R, E758R, and E761R; (25) I757R, E758R, and K768R; (26) 1757R, E761R and K768R; (27) E758R, E761R, and K768R; (28) I757R, E758R, E761R, and K768R (29) N865W; (30) N865Y; (31) Q866M; (32) Q869M; (33) Q1093W; (34) Q1093Y; and/or (35) Q866M and Q869M; wherein the amino acid position number is in reference to SEQ ID NO: 1.

Embodiment 31. An engineered Cas12b nuclease comprising the amino acid sequence of any one of SEQ ID NOs: 2-22.

Embodiment 32. An engineered Cas12b effector protein comprising the engineered Cas12b nuclease according to any one of embodiments 1-31 or a functional derivative thereof.

Embodiment 33. The engineered Cas12b effector protein of embodiment 32, wherein the engineered Cas12b nuclease or a functional derivative thereof is enzymatically active.

Embodiment 34. The engineered Cas12b effector protein of embodiment 32 or 33, wherein the engineered Cas12b effector protein is capable of inducing a double-strand break in a DNA molecule.

Embodiment 35. The engineered Cas12b effector protein of embodiment 32 or 33, wherein the engineered Cas12b effector protein is capable of inducing a single-strand break in a DNA molecule.

Embodiment 36. The engineered Cas12b effector protein of embodiment 32, wherein the engineered Cas12b effector protein comprises an enzymatically inactive mutant of the engineered Cas12b nuclease.

Embodiment 37. The engineered Cas12b effector protein of embodiment 36, wherein the enzyme-inactivating mutant comprises D570A, R785A, E848A, R911A, and/or D977A.

Embodiment 38. The engineered Cas12b effector protein of any one of embodiments 32-37, further comprising a functional domain fused to the engineered Cas12b nuclease or functional derivative thereof.

Embodiment 39. The engineered Cas12b effector protein of embodiment 38, wherein the functional domain is selected from the group consisting of a translation initiator domain, a transcription repressor domain, a transactivation domain, an epigenetic modification domain, a nucleobase-editing domain, a reverse transcriptase domain, a reporter domain, and a nuclease domain.

Embodiment 40. The engineered Cas12b effector protein of any one of embodiments 32-37, comprising a first polypeptide comprising an N-terminal portion of the engineered Cas nuclease or functional derivative thereof, and a second polypeptide comprising a C-terminal portion of the engineered Cas nuclease or functional derivative thereof, wherein the first polypeptide and the second polypeptide are capable of associating with each other in the presence of a guide RNA comprising a guide sequence to form a Clustered Regularly Interspersed Short Palindromic Repeat (CRISPR) complex that specifically binds to a target nucleic acid comprising a target sequence complementary to the guide sequence.

Embodiment 41. The engineered Cas12b effector protein of embodiment 40, comprising a first polypeptide and a second polypeptide, wherein the first polypeptide comprises the N-terminal amino acid residues 1 to X of the engineered Cas12b nuclease or functional derivative thereof, wherein the second polypeptide comprises the X+1 residue to the C-terminus of the engineered Cas12b nuclease or functional derivative thereof, wherein the first polypeptide and the second polypeptide are capable of associating with each other in the presence of a guide RNA containing a guide sequence to form a clustered regular interval short palindromic repeat (CRISPR) complex that specifically binds to a target nucleic acid, wherein the target nucleic acid comprises a target sequence complementary to the guide sequence.

Embodiment 42. The engineered Cas12b effector protein of embodiment 40 or 41, wherein the first polypeptide and the second polypeptide each comprises a dimerization domain.

Embodiment 43. The engineered Cas12b effector protein of embodiment 42, wherein the first dimerization domain and the second dimerization domain associate with each other in the presence of an inducer.

Embodiment 44. The engineered Cas12b effector protein of embodiment 40 or 41, wherein the first polypeptide and the second polypeptide do not comprise dimerization domains.

Embodiment 45. An engineered CRISPR-Cas12b system comprising: (a) the engineered Cas12b effector protein of any one of embodiments 32-44, or a nucleic acid encoding the engineered Cas12b effector protein; and (b) a guide RNA comprising a guide sequence complementary to a target sequence, or a nucleic acid encoding the guide RNA, wherein the engineered Cas12b effector protein and the guide RNA are capable of forming a CRISPR complex that specifically binds to a target nucleic acid comprising the target sequence and inducing a modification of the target nucleic acid.

Embodiment 46. The engineered CRISPR-Cas12b system of embodiment 45, wherein the guide RNA comprises a crRNA and a tracrRNA.

Embodiment 47. The engineered CRISPR-Cas12b system of embodiment 45 or 46, comprising a precursor guide RNA array encoding a plurality of crRNAs.

Embodiment 48. The engineered CRISPR-Cas12b system of any one of embodiments 45-47, wherein the guide RNA is a single guide RNA (sgRNA).

Embodiment 49. The engineered CRISPR-Cas12b system of any one of embodiments 45-48, comprising one or more vectors encoding the engineered Cas12b effector protein.

Embodiment 50. The engineered CRISPR-Cas12b system of embodiment 49, wherein the one or more vector is an adeno-associated viral (AAV) vector.

Embodiment 51. The engineered CRISPR-Cas12b system of embodiment 50, wherein the AAV vector further encodes the guide RNA.

Embodiment 52. A method of detecting target nucleic acid in a sample, including: (a) contacting the sample with the engineered CRISPR-Cas12b system of any one of embodiments 45-51 and a labeled detector nucleic acid, wherein the labeled detector nucleic acid is single-stranded and does not hybridize with the guide sequence of the guide RNA; and (b) measuring a detectable signal produced by cleavage of the labeled detector nucleic acid by the engineered Cas12b effector protein, thereby detecting the target nucleic acid.

Embodiment 53. A method of modifying a target nucleic acid comprising a target sequence, comprising contacting the target nucleic acid with the engineered CRISPR-Cas12b system of any one of embodiments 45-51.

Embodiment 54. The method of embodiment 53, wherein the method is carried out in vitro.

Embodiment 55. The method of embodiment 53, wherein the target nucleic acid is present in a cell.

Embodiment 56. The method of embodiment 55, wherein the cell is a bacterial cell, a yeast cell, a mammalian cell, a plant cell, or an animal cell.

Embodiment 57. The method of embodiment 53, wherein the method is carried out ex vivo.

Embodiment 58. The method of embodiment 53, wherein the method is carried out in vivo.

Embodiment 59. The method of any one of embodiments 53-58, wherein the target nucleic acid is cleaved or the target sequence in the target nucleic acid is altered by the engineered CRISPR-Cas12b system.

Embodiment 60. The method of any one of embodiments 53-58, wherein expression of the target nucleic acid is altered by the engineered CRISPR-Cas12b system.

Embodiment 61. The method of any one of embodiments 53-60, wherein the target nucleic acid is a genomic DNA.

Embodiment 62. The method of any one of embodiments 53-61, wherein the target sequence is associated with a disease or condition.

Embodiment 63. The method of any one of embodiments 53-62, wherein the engineered CRISPR-Cas12b system comprises a precursor guide RNA array encoding a plurality of crRNA, wherein each crRNA comprises a different guide sequence.

Embodiment 64. A method of treating a disease or condition associated with a target nucleic acid in cells of an individual, comprising modifying the target nucleic acid in the cells of the individual using the engineered CRISPR-Cas12b system of any one of embodiments 45-51, thereby treating the disease or condition.

Embodiment 65. The method of embodiment 64, wherein the disease or condition is selected from the group consisting of cancer, cardiovascular diseases, hereditary diseases, autoimmune diseases, metabolic diseases, neurodegenerative diseases, ocular diseases, bacterial infections and viral infections.

Embodiment 66. An engineered cell comprising a modified target nucleic acid, wherein the target nucleic acid has been modified using the method of any one of embodiments 53-63.

Embodiment 67. An engineered non-human animal comprising one or more engineered cells of embodiment 66.

### EXAMPLES

The examples below are intended to be purely exemplary of the invention and should therefore not be considered to limit the invention in any way. The following examples and detailed description are offered by way of illustration and not by way of limitation.

### Methods

### Construction of Plasmids

The coding sequence of AaCas12b was codon-optimized for expression in human cells and synthesized. Nucleic acid sequences encoding the engineered AaCas12b protein mutants were produced by PCR-based site-directed mutagenesis. Specifically, the DNA sequence encoding a reference AaCas12b protein was divided into two parts centered on a mutation site. Two pairs of primers were designed to amplify the two parts of the DNA sequence and assembled into a single piece of DNA by Gibson cloning, which was incorporated into the pCAG-2A-eGFP vector. Combinations of mutations were constructed by splitting the DNA encoding a reference AaCas12b protein into multiple segments, amplified and assembled using PCR and Gibson cloning. The DNA encoding the engineered AaCas12b protein was inserted between XmaI and NheI sites of the pCAG-2A-eGFP vector. The positions of the mutations in the AaCas12b protein variants were designed based on analysis of the crystal structure of AaCas12b using protein structure visualization software commonly used in the field (for example, PyMol, or Chimera). Crystal structures of AaCas12b are available at RCSB PDB database with access numbers, 6LTU, 6LTR, 6LU0, and 6LTP. The AaCas12b variants were expressed in human 293T cells using the pCAG-2A-eGFP vector. DNA sequences encoding sgRNA scaffolds were de novo synthesized and assembled into pUC19-U6 backbone via Gibson clone. Nucleic acid encoding the spacer sequence was also ligated into the same pUC19-U6 backbone.

### Cell Culture, Transfection and Fluorescence Activated Cell Sorting (FA CS)

HEK293T cells were cultured in DMEM (Gibco) containing 1% penicillin-streptomycin (Gibco) and 10% fetal bovine serum (Gibco). The cells were seeded in a 24-well culture dish (Corning) for 16 hours until the confluence reached 70%. Using Lipofectamine 3000 (Invitrogen), 600 ng of the plasmid encoding an AaCas12b protein and various amounts of the plasmid encoding sgRNA were transfected into cells in each well of the 24-well culture dish. After 68 hours of transfection, the HEK293T cells were digested by Trypsin-EDTA (0.05%) (Gibco), and subject to FACS sorting using MoFlo XDP (Beckman Coulter) based on GFP signal (indicating successful transfection).

### Targeted Deep Sequencing Analysis for Genome Modification

The GFP-positive HEK293T cells sorted by FACS were lysed with buffer L and incubated at 55° C. for 3 hours, and then at 95° C. for 10 minutes. The corresponding primers were used for PCR amplification of dsDNA fragments containing target sites at different genomic loci. For targeted deep sequencing, the cell lysates was directly used as template DNA for amplification by barcoded PCR. The PCR products were purified and pooled into several libraries for high-throughput sequencing. By calculating the ratio of reads containing insertions or deletions, the frequency (%) of indels was analyzed using CRISPResso2 software. In this application, the index of indel frequency (%) was used to compare and analyze gene editing efficiency for different engineered Cas12b proteins and/or in the presence of different sgRNA scaffolds. Any number of reads fewer than 0.05% of the total reads was discarded.

### Example 1: Substitution of One or More Amino Acid Residues in the Reference AaCas12b Nuclease that Interact with PAM with a Positively Charged Amino Acid Residue

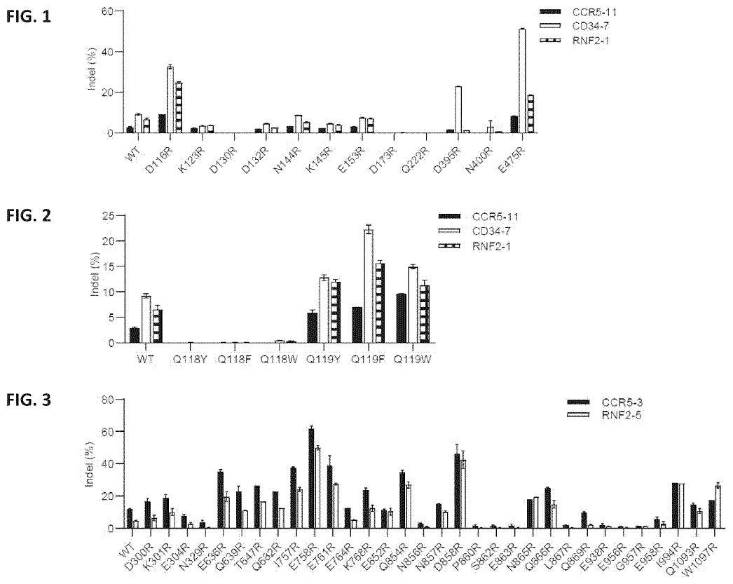

Engineered AaCas12b enzymes with a single mutation in the amino acid residues that interact with PAM were designed and expressed according to the method described above. Briefly, 10 amino acids within 9 A of PAM in AaCas12b were selected: D116, K123, D130, D132, N144, K145, E153, D173, Q222, D395, N400, and E475, and substituted each amino acid residue with an arginine (R). Nucleic acids encoding sgRNAs against target sites CCR5-11 (SEQ ID NO: 63), CD34-7 (SEQ ID NO: 64), and RNF2-1 (SEQ ID NO: 65) were designed, which comprise from 5′ to 3′: DNA encoding Aa-sg sgRNA scaffold sequence (SEQ ID NO: 23)—DNA encoding spacer sequence, and cloned into pUC19-U6 backbone. Using Lipofectamine 3000 (Invitrogen), 600 ng of the plasmid encoding an AaCas12b protein and 300 ng of the plasmid encoding sgRNA were transfected into HEK293T cells in each well of a 24-well culture dish as described above. Wild-type AaCas12b (SEQ ID NO: 1) served as control. The amino acid substitutions in AaCas12b enzymes and the corresponding gene editing efficiencies are shown in FIG. 1 and Table 1. AaCas12b variants with amino acid substitution D116R (SEQ ID NO: 2) or E475R (SEQ ID NO: 3) displayed improved gene editing efficiency compared to wild-type AaCas12b. As shown in FIG. 1, AaCas12b-D116R (SEQ ID NO: 2) and AaCas12b-E475R (SEQ ID NO: 3) had an average of more than about 20% gene editing efficiency across the three genomic sites, as compared to an average of about 6% gene editing efficiency by the reference wild-type AaCas12b nuclease. The indel frequencies of the AaCas12b-D116R (SEQ ID NO: 2) and AaCas12b-E475R (SEQ ID NO: 3) mutants were significantly higher than those using other AaCas12b mutants tested in this category. AaCas12b-D395R achieved much higher gene editing efficiency at CD34-7 locus compared to wildtype AaCas12b, but not at other tested loci.

TABLE 1
Gene editing efficiency at different loci for different AaCas12b

| AaCas12b variants | Indel (%) at CCR5-11 | Indel (%) at CD34-7 | Indel (%) at RNF2-1 |
|---|---|---|---|
| WT (SEQ ID NO: 1) | 2.87 | 9.20 | 6.60 |
| D116R (SEQ ID NO: | 9.09 | 32.75 | 24.74 |
| 2) | | | |
| K123R | 2.28 | 3.59 | 3.73 |
| D130R | 0.00 | 0.00 | 0.00 |
| D132R | 1.83 | 4.56 | 2.58 |
| N144R | 3.25 | 8.67 | 5.33 |
| K145R | 2.18 | 4.68 | 3.70 |
| E153R | 3.00 | 7.42 | 7.09 |
| D173R | 0.11 | 0.12 | 0.15 |
| Q222R | 0.00 | 0.07 | 0.00 |
| D395R | 1.54 | 22.84 | 1.37 |
| N400R | 0.04 | 2.96 | 0.59 |
| E475R (SEQ ID NO: 3) | 8.15 | 51.42 | 18.70 |

### Example 2: Substitution of One or More Amino Acid Residues in the Reference AaCas12b Nuclease that are Involved in Opening DNA Double Strands with an Amino Acid Residue Having an Aromatic Ring

Engineered AaCas12b nucleases with a single substitution in the amino acid residues that are involved in opening DNA double strands were designed and expressed according to the method described above. Briefly, amino acid residue Q118 or Q119 was substituted with an aromatic amino acid residue (e.g., Y, F, or W). Same sgRNA encoding plasmids in Example 1 were used here. Using Lipofectamine 3000 (Invitrogen), 600 ng of the plasmid encoding an AaCas12b protein and 300 ng of the plasmid encoding sgRNA were transfected into HEK293T cells in each well of a 24-well culture dish as described above. Wild-type AaCas12b (SEQ ID NO: 1) served as control. The amino acid substitutions in AaCas12b enzymes and the corresponding gene editing efficiencies are shown in FIG. 2 and Table 2. AaCas12b with the amino acid substitution Q119Y, Q119F, or Q119W displayed improved gene editing efficiencies compared to wild-type AaCas12b at all tested loci. The indel frequencies of the AaCas12b-Q119Y, AaCas12b-Q119F and AaCas12b-Q119W mutants were significantly higher than those using other AaCas12b mutants (Q118Y, Q118F, Q118W) tested in this category.

TABLE 2
Gene editing efficiency at different loci for different AaCas12b

| AaCas12b variants | Indel (%) at CCR5-11 | Indel (%) at CD34-7 | Indel (%) at RNF2-1 |
|---|---|---|---|
| WT (SEQ ID NO: 1) | 2.87 | 9.20 | 6.60 |
| Q118Y | 0.00 | 0.07 | 0.00 |
| Q118F | 0.03 | 0.09 | 0.04 |
| Q118W | 0.08 | 0.41 | 0.29 |
| Q119Y (SEQ ID NO: 4) | 5.84 | 12.77 | 12.00 |
| Q119F (SEQ ID NO: 5) | 6.98 | 22.28 | 15.66 |
| Q119W (SEQ ID NO: 6) | 9.56 | 14.95 | 11.32 |

### Example 3: Substitution of One or More Amino Acid Residues in the RuvC Domain of the Reference AaCas12b Nuclease that Interact with a Single-Stranded DNA Substrate with a Positively Charged Amino Acid Residue or a Hydrophobic Amino Acid Residue

Engineered AaCas12b nucleases with a single amino acid substitution in the amino acid residues in the RuvC domain that interact with a single-stranded DNA substrate were designed and expressed according to the method described above. Nucleic acids encoding sgRNAs against target sites CCR5-3 (SEQ ID NO: 66) and RNF2-5 (SEQ ID NO: 67) were designed, which comprise from 5′ to 3′: DNA encoding Aa-sg sgRNA scaffold sequence (SEQ ID NO: 23)—DNA encoding spacer sequence, and cloned into pUC19-U6 backbone. Using Lipofectamine 3000 (Invitrogen), 600 ng of the plasmid encoding an AaCas12b protein and 300 ng of the plasmid encoding sgRNA were transfected into HEK293T cells in each well of a 24-well culture dish. Wild-type AaCas12b (SEQ ID NO: 1) served as control.

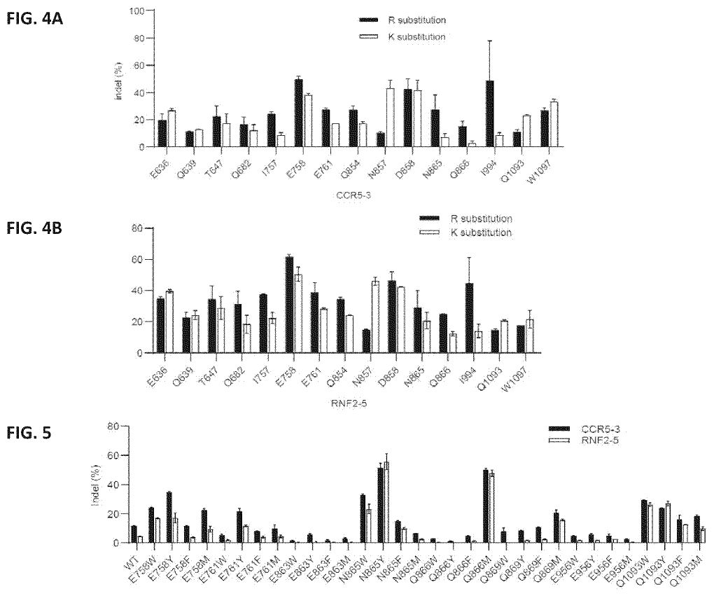

In a first group of AaCas12b mutants, each of the amino acid residues in Table 3 was substituted with a positively charged amino acid residue arginine (R). The amino acid substitutions in AaCas12b enzymes and the corresponding gene editing efficiencies are shown in FIGS. 3-4B and Table 3.

TABLE 3
Gene editing efficiency at different loci for different AaCas12b

| AaCas12b variants | Indel (%) at CCR5-3 | Indel (%) at RNF2-5 |
|---|---|---|
| WT | 11.54 | 4.63 |
| D300R | 16.50 | 6.38 |
| K301R | 18.83 | 9.84 |
| E304R | 7.37 | 2.79 |
| N329R | 3.56 | 0.32 |
| E636R (SEQ ID NO: 7) | 34.92 | 19.54 |
| Q639R | 22.75 | 11.00 |
| T647R | 26.25 | 16.53 |
| Q682R | 22.73 | 12.47 |
| I757R (SEQ ID NO: 8) | 37.69 | 24.15 |
| E758R (SEQ ID NO: 9) | 61.70 | 49.84 |
| E761R (SEQ ID NO: 10) | 38.78 | 27.33 |
| E764R | 12.18 | 5.14 |
| K768R | 23.53 | 12.28 |
| E852R | 11.17 | 10.33 |
| Q854R (SEQ ID NO: 11) | 34.66 | 26.88 |
| N856R | 2.66 | 0.79 |
| N857R | 14.85 | 10.22 |
| D858R (SEQ ID NO: 12) | 46.16 | 42.42 |
| [symbol] 860R | 1.22 | 0.13 |
| S862R | 1.49 | 0.30 |
| E863R | 1.48 | 0.17 |
| N865R | 17.78 | 19.48 |
| Q866R | 24.89 | 14.88 |
| L867R | 1.72 | 0.18 |
| Q869R | 9.49 | 2.11 |
| E938R | 1.92 | 1.17 |
| E956R | 0.81 | 0.29 |
| G957R | 1.37 | 1.00 |
| E958R | 5.65 | 2.97 |
| I994R | 28.14 | 27.87 |
| Q1093R | 14.68 | 10.62 |
| W1097R | 17.38 | 26.37 |

[symbol]

indicates data missing or illegible when filed

In a second group of AaCas12b mutants, each of the amino acid residues in Table 4 was substituted with a positively charged amino acid residue lysine (K). The amino acid substitutions in AaCas12b enzymes and the corresponding gene editing efficiencies are shown in Table 4 and FIGS. 4A-4B.

As shown in Tables 3-4 and FIGS. 3-4B, AaCas12b mutants with the amino acid substitution D300R, K301R, E636R, Q639R, T647R, Q682R, I757R, E758R, E761R, K768R, Q854R, N857R, D858R, N865R, Q866R, 1994R, Q1093R, W1097R, E636K, Q639K, T647K, Q682K, 1757K, E758K, E761K, Q854K, N857K, D858K, N865K, 1994K, Q1093K, or W1097K had improved gene editing efficiency compared to wild-type AaCas12b at both tested loci. The indel frequencies of AaCas12b E636R, I757R, E758R, E761R, Q854R, D858R, E758K, N857K, I994R, and D858K mutants were significantly higher than those using other AaCas12b mutants tested in this category (substitute with positively charged amino acid residue).

TABLE 4
Gene editing efficiency at different loci for different AaCas12b

| AaCas12b variants | Indel (%) at CCR5-3 | Indel (%) at RNF2-5 |
|---|---|---|
| WT | 11.54 | 4.63 |
| E636K | 39.58 | 26.45 |
| Q639K | 24.23 | 12.68 |
| T647K | 28.84 | 17.31 |
| Q682K | 18.46 | 12.10 |
| I757K | 22.40 | 8.53 |
| E758K | 50.37 | 38.27 |
| E761K | 28.20 | 16.90 |
| Q854K | 24.24 | 17.08 |
| N857K (SEQ ID NO: 13) | 45.97 | 43.19 |
| D858K | 42.27 | 41.78 |
| N865K | 20.81 | 7.27 |
| Q866K | 12.39 | 2.70 |
| I994K | 14.20 | 8.66 |
| Q1093K | 20.60 | 23.15 |
| W1097K | 21.66 | 33.05 |

In a third group of AaCas12b mutants, each of the following amino acid residues was substituted with a hydrophobic amino acid residue (e.g., Y, F, M, or W): E758, E761, E863, N865, Q866, Q869, E956, and Q1093. The amino acid substitutions in AaCas12b enzymes and the corresponding gene editing efficiencies are shown in FIG. 5 and Table 5. AaCas12b mutants with the amino acid substitution E758W, E758Y, E758M, E761Y, N865W, N865Y, N865F, Q866M, Q869M, Q1093W, Q1093Y, Q1093F, or Q1093M displayed improved gene editing efficiency compared to wild-type AaCas12b at both tested loci. The indel frequencies of the AaCas12b N865W, N865Y, Q866M, Q869M, Q1093W, and Q1093Y mutants were significantly higher than the other AaCas12b mutants tested in this category (substitution with an hydrophobic amino acid residue).

TABLE 5
Gene editing efficiency at different loci for different AaCas12b

| AaCas12b variants | Indel (%) at CCR5-3 | Indel (%) at RNF2-5 |
|---|---|---|
| WT | 11.54 | 4.63 |
| E758W | 24.31 | 17.13 |
| E758Y | 34.73 | 17.20 |
| E758F | 11.57 | 3.87 |
| E758M | 22.39 | 9.52 |
| E761W | 5.17 | 1.87 |
| E761Y | 21.47 | 11.78 |
| E761F | 8.02 | 4.06 |
| E761M | 9.86 | 4.55 |
| E863W | 1.48 | 0.36 |
| E863Y | 5.58 | 0.52 |
| E863F | 1.49 | 0.35 |
| E863M | 2.86 | 0.48 |
| N865W (SEQ ID NO: 14) | 32.50 | 23.18 |
| N865Y (SEQ ID NO: 15) | 51.61 | 55.65 |
| N865F | 14.85 | 9.85 |
| N865M | 6.25 | 2.49 |
| Q866W | 2.95 | 0.27 |
| Q866Y | 1.24 | 0.27 |
| Q866F | 4.71 | 1.47 |
| Q866M (SEQ ID NO: 16) | 50.09 | 47.76 |
| Q869W | 7.93 | 0.61 |
| Q869Y | 8.47 | 1.83 |
| Q869F | 10.70 | 2.57 |
| Q869M (SEQ ID NO: 17) | 20.65 | 15.71 |
| E956W | 4.73 | 1.88 |
| E956Y | 5.57 | 1.71 |
| E956F | 4.90 | 2.85 |
| E956M | 2.66 | 0.59 |
| Q1093W (SEQ ID NO: 18) | 29.32 | 26.26 |
| Q1093Y (SEQ ID NO: 19) | 23.73 | 27.12 |
| Q1093F | 15.97 | 12.65 |
| Q1093M | 18.14 | 9.92 |

### Example 4: Combinations of Mutations from Examples 1-3 and Characterization of their Gene Editing Efficiencies

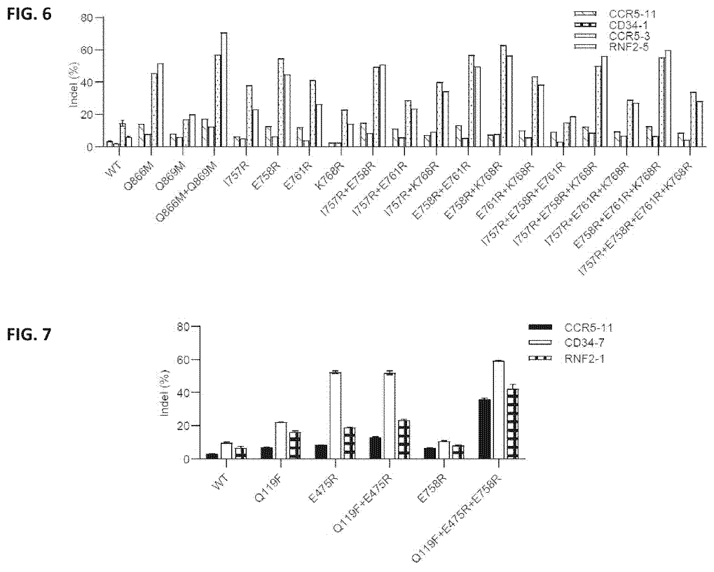

Amino acid substitutions screened in Examples 1, 2, and 3 with desired gene editing efficiencies, namely Q866M, Q869M, I757R, E758R, E761R, K768R, and 1757R, to make AaCas12b proteins with multiple mutations, namely Q866M+Q869M, I757R+E758R, I757R+E761R, I757R+K768R, E758R+E761R, E758R+K768R, E761R+K768R, I757R+E758R+E761R, I757R+E758R+K768R, I757R+E761R+K768R, E758R+E761R+K768R, and I757R+E758R+E761R+K768R. Nucleic acids encoding sgRNAs against target sites CCR5-3 (SEQ ID NO: 66), CCR5-11 (SEQ ID NO: 63), CD34-1 (SEQ ID NO: 68), and RNF2-5 (SEQ ID NO: 67) were designed, which comprise from 5′ to 3′: DNA encoding Aa-sg sgRNA scaffold sequence (SEQ ID NO: 23)—DNA encoding spacer sequence, and cloned into pUC19-U6 backbone. Wild-type AaCas12b (SEQ ID NO: 1) served as control. Using Lipofectamine 3000 (Invitrogen), 600 ng of the plasmid encoding an AaCas12b protein as named above and 300 ng of the plasmid encoding sgRNA were transfected into HEK293T cells in each well of a 24-well culture dish. Their gene editing efficiencies are shown in FIG. 6 and Table 6. AaCas12b mutants with a combination of amino acid substitutions all displayed significantly improved gene editing efficiencies compared to wild-type AaCas12b at all tested loci. Certain AaCas12b combination mutants, such as Q866M+Q869M, E758R+E761R, E758R+E768R, I757R+E758R+K768R, and E758R+E761R+K768R, have improved gene editing efficiencies at certain loci compared to corresponding single mutants.

TABLE 6
Gene editing efficiency at different loci for different AaCas12b

| | Indel (%) at | Indel (%) at | Indel (%) at | Indel (%) at |
|---|---|---|---|---|
| AaCas12b variants | CCR5-3 | CCR5-11 | CD34-1 | RNF2-5 |
| WT | 14.52 | 3.42 | 2.16 | 6.17 |
| Q866M (SEQ ID NO: 16) | 45.78 | 14.21 | 7.98 | 51.87 |
| Q869M (SEQ ID NO: 17) | 17.16 | 8.03 | 6.11 | 20.01 |
| Q866M + Q869M | 56.97 | 17.48 | 12.39 | 70.75 |
| (SEQ ID NO: 20) | | | | |
| I757R (SEQ ID NO: 8) | 38.29 | 6.51 | 5.29 | 23.37 |
| E758R (SEQ ID NO: 9) | 54.69 | 12.97 | 6.55 | 44.75 |
| E761R (SEQ ID NO: 10) | 41.32 | 12.22 | 4.17 | 26.48 |
| K768R | 23.14 | 2.61 | 2.76 | 14.33 |
| I757R + E758R | 49.55 | 14.89 | 8.48 | 50.96 |
| I757R + E761R | 28.80 | 11.21 | 5.94 | 23.57 |
| I757R + K768R | 40.14 | 7.24 | 9.45 | 34.32 |
| E758R + E761R | 56.90 | 13.48 | 5.64 | 49.74 |
| E758R + K768R | 62.90 | 7.57 | 7.85 | 56.64 |
| E761R + K768R | 43.56 | 10.26 | 5.86 | 38.56 |
| I757R + E758R + E761R | 15.24 | 9.45 | 3.26 | 19.00 |
| I757R + E758R + K768R | 50.13 | 12.45 | 8.93 | 56.24 |
| I757R + E761R + K768R | 29.07 | 9.77 | 6.83 | 27.37 |
| E758R + E761R + K768R | 55.44 | 12.94 | 6.63 | 60.03 |
| I757R + E758R + E761R + K768R | 34.10 | 8.81 | 4.56 | 28.23 |

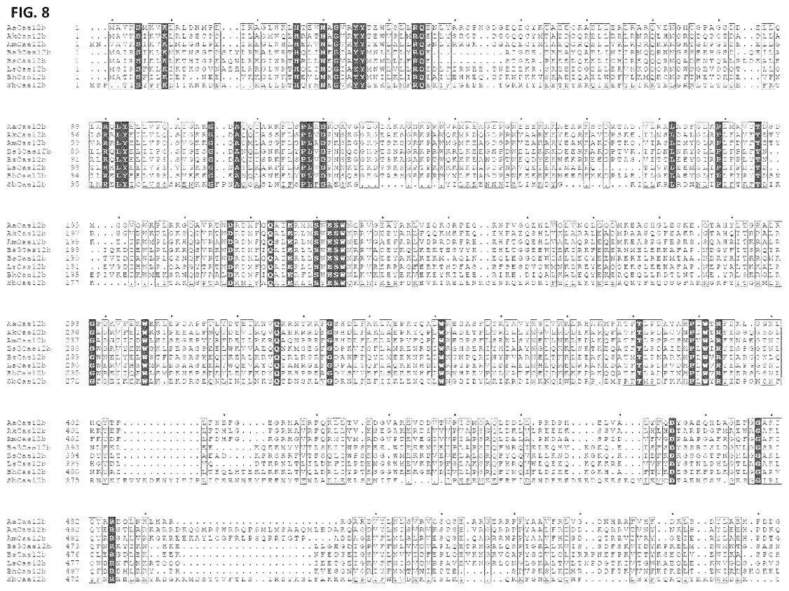

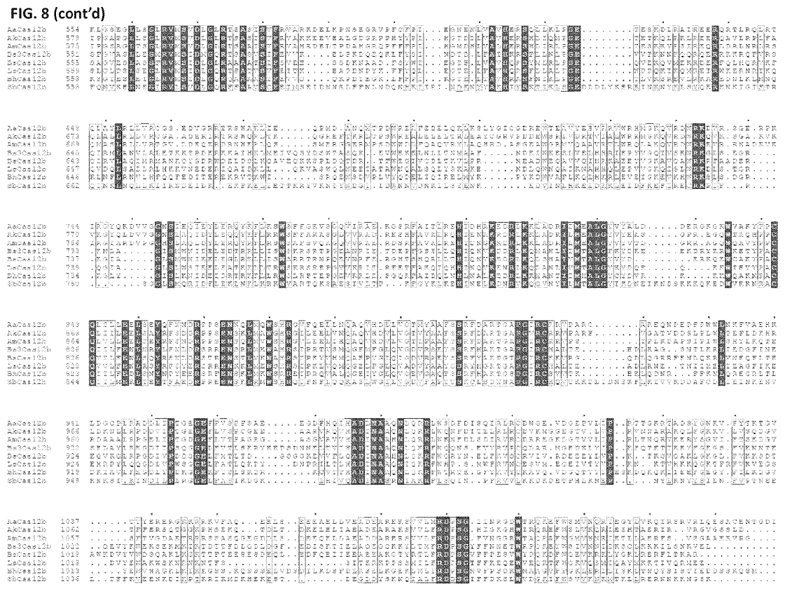

AaCas12b-Q119F+E475R and AaCas12b-Q119F+E475R+E758R were generated as described above. Same sgRNA encoding plasmids in Example 1 were used here. Wild-type AaCas12b (SEQ ID NO: 1) served as control. Using Lipofectamine 3000 (Invitrogen), 600 ng of the plasmid encoding an AaCas12b protein as named above and 300 ng of the plasmid encoding sgRNA were transfected into HEK293T cells in each well of a 24-well culture dish. Their gene editing efficiencies are shown in FIG. 7 and Table 7. Results showed that both AaCas12b-Q119F+E475R and AaCas12b-Q119F+E475R+E758R significantly improved gene editing efficiencies compared to wild-type AaCas12b at all tested loci. AaCas12b-Q119F+E475R+E758R displayed the most significant improvement in the gene editing efficiency with respect to the wild-type AaCas12b or corresponding AaCas12b variant with single substitution at all tested loci: CCR5-11, CD34-7, and RNF2-1.

TABLE 7
Gene editing efficiency at different loci for different AaCas12b

| AaCas12b variants | Indel (%) at CCR5-11 | Indel (%) at CD34-7 | Indel (%) at RNF2-1 |
|---|---|---|---|
| WT | 3.06 | 9.71 | 6.72 |
| Q119F (SEQ ID NO: 5) | 7.02 | 22.02 | 16.21 |
| E475R (SEQ ID NO: 3) | 8.29 | 52.13 | 18.87 |
| Q119F + E475R | 12.95 | 51.89 | 23.39 |
| (SEQ ID NO: 21) | | | |
| E758R (SEQ ID NO: 9) | 6.46 | 10.87 | 8.31 |
| Q119F + E475R + E758R | 35.68 | 59.39 | 42.22 |
| (SEQ ID NO: 22) | | | |

### Example 5: Enhancement of Gene Editing Activity of Engineered AaCas12b Using sgRNA with Engineered Scaffold

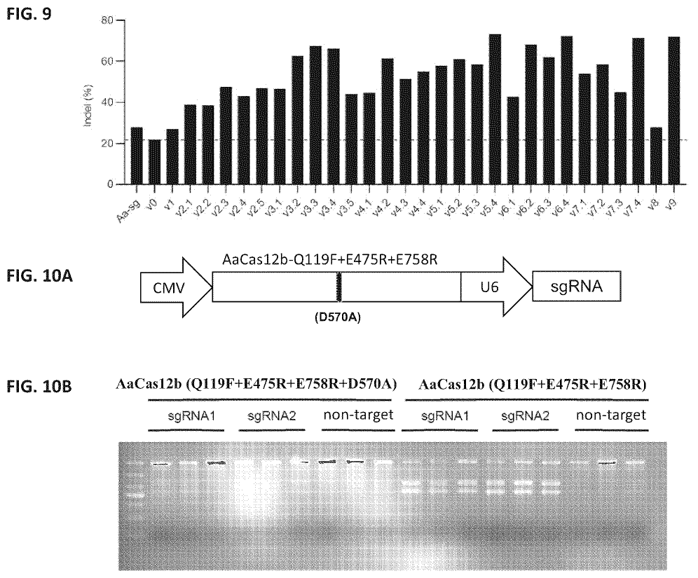

In this example, various sgRNAs with engineered scaffolds were tested for gene editing activity using the AaCas12b mutant from Example 4 (Q119F+E475R+E758R). Nucleic acid encoding sgRNA against target site CCR5-11 (SEQ ID NO: 63) was designed, which comprise from 5′ to 3′: DNA encoding sgRNA scaffold sequence—DNA encoding spacer sequence, and cloned into pUC19-U6 backbone. Using Lipofectamine 3000 (Invitrogen), 600 ng of the plasmid encoding the AaCas12b variant protein and 300 ng of a plasmid encoding sgRNA with engineered scaffold (SEQ ID NOs: 25-53; modified based off AacCas12b sgRNA scaffold V0), AacCas12b sgRNA scaffold (SEQ ID NO: 24; V0, control; H. Yang et al., Cell. 2016; 167(7):1814-1828.e12), or AaCas12b Aa-sg scaffold (SEQ ID NO: 23; control) were transfected into HEK293T cells in each well of a 24-well culture dish. Their gene editing efficiencies are shown in FIG. 9. Data in FIG. 9 showed that all of the sgRNA \hat{ }ngineered scaffolds significantly improved gene editing efficiencies of AaCas12b (Q119F+E475R+E758R) variant compared to the AacCas12b sgRNA scaffold (V0). All of the sgRNA engineered scaffolds (except for V1 and V8), also significantly improved gene editing efficiencies of AaCas12b (Q119F+E475R+E758R) variant compared to the Aa-sg scaffold.

### Example 6: Engineered AaCas12b with Inactivated Nuclease Activity

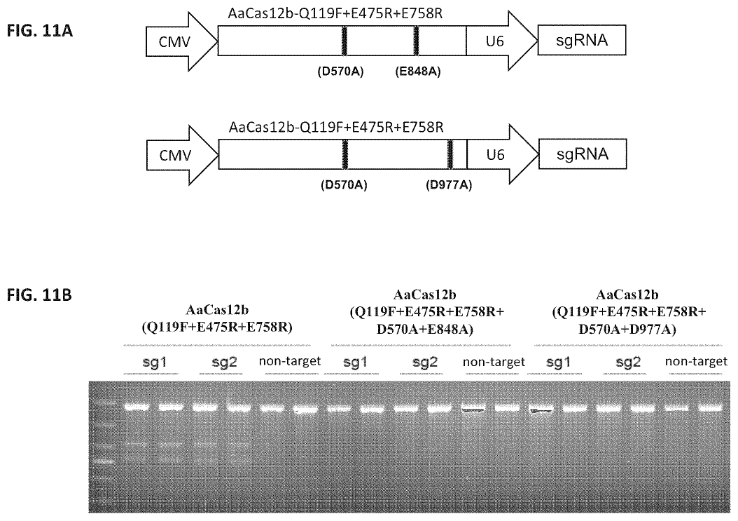

To generate a deactivated AaCas12b protein, the AaCas12b (Q119F+E475R+E758R) variant (SEQ ID NO: 22) from Example 4 were further modified to comprise an additional single point mutation in the nucleolytic domain (D570A) (FIG. 10A). A plasmid co-encoding i) AaCas12b (Q119F+E475R+E758R) or AaCas12b (Q119F+E475R+E758R+D570A) (SEQ ID NO: 79) under the control of a CMV promoter, and ii) a control sgRNA (not targeting any sequence within hemoglobin subunit gamma 1/2 (HBG1/2)), sgRNA1 (against target sequence SEQ ID NO: 70 of HBG1/2), or sgRNA2 (against target sequence SEQ ID NO: 71 of HBG1/2) under the control of a U6 promoter, was transfected into HEK293 cells (Table 8; see FIG. 10A for plasmid construction), using similar methods as described above. SgRNA scaffold (V9) (SEQ ID NO: 53) was used to construct these sgRNAs. 3 days post-transfection, genomic DNA was extracted from the transfected cells. T7 endonuclease I (T7EI) mismatch detection assay was performed to determine the cleavage efficiency (M. Crispo et al., PLoS One. 2015; 10(8):e0136690). The primer sequences used in T7EI assay are listed in Table 9.

As shown in FIG. 10B, the catalytic activity of AaCas12b (Q119F+E475R+E758R+D570A) was dramatically reduced compared to AaCas12b (Q119F+E475R+E758R) in cleaving two different target sites of HBG1/2 guided by either sgRNA1 or sgRNA2.

TABLE 8
PAM and target site for sgRNAs
targeting HBG1/2

| sgRNA | PAM | Target sequence |
|---|---|---|
| sgRNA1 | TTG | AGATAGTGTGGGGAAGGGGC |
| | | (SEQ ID NO: 70) |
| sgRNA2 | TTT | GCATTGAGATAGTGTGGGGA |
| | | (SEQ ID NO: 71) |

TABLE 9
Primer sequences used in T7EI assay

| SEQ ID NO | Primer sequence |
|---|---|
| 69 | TCCTGCACTGAAACTGTTGC |
| 78 | TCCTGAGAAGCGACCTGGA |

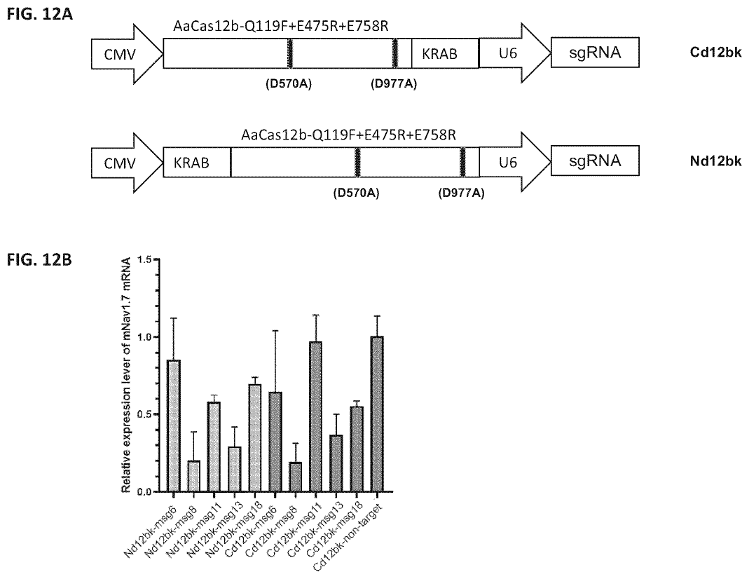

To further reduce the nuclease activity of the engineered AaCas12b, an additional point mutation was introduced into AaCas12b (Q119F+E475R+E758R+D570A) to generate AaCas12b (Q119F+E475R+E758R+D570A+E848A) (SEQ ID NO: 80) or AaCas12b (Q119F+E475R+E758R+D570A+D977A) (SEQ ID NO: 81) (FIG. 11A). A plasmid co-encoding i) AaCas12b (Q119F+E475R+E758R), AaCas12b (Q119F+E475R+E758R+D570A+E848A), or AaCas12b (Q119F+E475R+E758R+D570A+D977A) under the control of a CMV promoter, and ii) sgRNA1 (against target sequence SEQ ID NO: 70 of HBG1/2) or sgRNA2 (against target sequence SEQ ID NO: 71 of HBG1/2) under the control of a U6 promoter, was transfected into HEK293 cells (see FIG. 11A for plasmid construction), using similar methods as described above. As negative control, a plasmid encoding AaCas12b (Q119F+E475R+E758R), AaCas12b (Q119F+E475R+E758R+D570A+E848A), or AaCas12b (Q119F+E475R+E758R+D570A+D977A) and a control sgRNA (not targeting any sequence within hemoglobin subunit gamma 1/2 (HBG1/2) was similarly transfected into HEK293 cells without any sequence encoding sgRNA. As shown in FIG. 11B, both AaCas12b (Q119F+E475R+E758R+D570A+E848A) and AaCas12b (Q119F+E475R+E758R+D570A+D977A) completely abolished nuclease activity of AaCas12b (Q119F+E475R+E758R).

### Example 7: Transcription Repression Using an Engineered AaCas12b Fusion Protein

AaCas12b (Q119F+E475R+E758R+D570A+D977A) (SEQ ID NO: 81) from Example 6 was further engineered to generate a fusion protein to silence transcription of a target gene. AaCas12b (Q119F+E475R+E758R+D570A+D977A) (flanked by two copies of the nuclear localization sequence, NLS) was fused with a transcription repression module, Kruppel associated box (KRAB) domain of ZIM3 (SEQ ID NO: 72), which can recruit repressive chromatin modifiers. KRAB was either fused at the C-terminus of AaCas12b (Q119F+E475R+E758R+D570A+D977A) or at the N-terminus, and the fusion proteins were named Cd12bk and Nd12bk, respectively. The same plasmid also encodes an sgRNA specifically recognizing different target sites in SCN9A gene (encoding voltage-gated sodium channel 1.7 Naᵥ1.7) under the control of a U6 promoter (FIG. 12A; Table 10). SgRNA scaffold (V9) (SEQ ID NO: 53) was used to construct these sgRNAs.

To examine whether Cd12bk and Nd12bk fusion proteins could recruit chromatin-modifying complexes to silence transcription of SCN9A, the plasmid encoding the fusion proteins and an sgRNA was transfected into Neuro 2A (N2a; mouse neural crest-derived cell line) cells. As a control, a plasmid encoding Cd12bk with a control sgRNA (not targeting any sequence within SCN9A) was similarly transfected into N2a cells. 3 days post-transfection, the transfected cells were collected and RNA was extracted using RNA extraction Kit (Vazyme, Cat. No. RC112-01). The mRNA levels of Nav1.7 in each sample was determined by qPCR. Data was normalized to Cd12bk with the control sgRNA (“Cd12bk-non-target”). As shown in FIG. 12B, Cd12bk or Nd12bk together with sgRNA-msg6, sgRNA-msg8, sgRNA-msg13, or sgRNA-msg18 could greatly inhibit the transcription of SCN9A, with sgRNA-msg8 and sgRNA-msg13 showing the most inhibition. Nd12bk together with sgRNA-msg11 could also greatly inhibit the transcription of SCN9A. These results demonstrated that dAaCas12b, e.g., AaCas12b (Q119F+E475R+E758R+D570A+D977A), fused with KRAB can be used as a targeted transcriptional regulatory tool in eukaryotic cells.

TABLE 10
PAM and target site for sgRNAs targeting SCN9A

| sgRNA | PAM | Target site |
|---|---|---|
| msg6 | TTA | GCTGCCCGCCACACTGGCGC (SEQ ID NO: 73) |
| msg8 | TTG | GGCGTGGTGATGCTAGGGAT (SEQ ID NO: 74) |
| msg11 | TTC | TAGTCTGCTCAGGATGAAGC (SEQ ID NO: 75) |
| msg13 | TTC | AATCCTGCCCACTGTGCAGG (SEQ ID NO: 76) |
| msg18 | TTC | CCTTGGATCAGAATCCGCAG (SEQ ID NO: 77) |

Although the embodiments of the present invention have been described above with reference to the accompanying drawings, the present invention is not limited to the above specific embodiments and application fields. The above specific embodiments are only illustrative, instructive, and not restrictive. Under the enlightenment of this specification and without departing from the scope of protection of the claims of the present invention, those of ordinary skill in the art can also make many forms, which all belong to the protection of the present invention.

EXEMPLARY SEQUENCES

| # | Sequences |
|---|---|
| SEQ ID NO: 1 | MAVKSMKVKLRLDNMPEIRAGLWKLHTEVN |
| Wild type | AGVRYYTEWLSLLRQENLYRRSPNGDGEQE |
| AaCas12b | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 2 | MAVKSMKVKLRLDNMPEIRAGLWKLHTEVN |
| AaCas12b-D116R | AGVRYYTEWLSLLRQENLYRRSPNGDGEQE |
| | CYKTAEECKAELLERLRARQVENGHCGPAG |
| | SDDELLQLARQLYELLVPQAIGAKGRAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 3 | MAVKSMKVKLRLDNMPEIRAGLWKLHTEVN |
| AaCas12b-E475R | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGREGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 4 | MAVKSMKVKLRLDNMPEIRAGLWKLHTEVN |
| AaCas12b-Q119Y | AGVRYYTEWLSLLRQENLYRRSPNGDGEQE |
| | CYKTAEECKAELLERLRARQVENGHCGPAG |
| | SDDELLQLARQLYELLVPQAIGAKGDAQYI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 5 | MAVKSMKVKLRLDNMPEIRAGLWKLHTEVN |
| AaCas12b-Q119F | AGVRYYTEWLSLLRQENLYRRSPNGDGEQE |
| | CYKTAEECKAELLERLRARQVENGHCGPAG |
| | SDDELLQLARQLYELLVPQAIGAKGDAQFI |
| | ARKFLSPLADKDAVGGLGIAKAGNKPRWVR |
| | MREAGEPGWEEEKAKAEARKSTDRTADVLR |
| | ALADFGLKPJ.MRVYTDSDMSSVQWKPLRK |
| | GQAVRTWDRDMFQQAIERMMSWESWNQRVG |
| | EAYAKLVEQKSRFEQKNFVGQEHLVQLVNQ |
| | LQQDMKEASHGLESKEQTAHYLTGRALRGS |
| | DKVFEKWEKLDPDAPFDLYDTEIKNVQRRN |
| | TRRFGSHDLFAKLAEPKYQALWREDASFLT |
| | RYAVYNSIVRKLNHAKMFATFTLPDATAHP |
| | IWTRFDKLGGNLHQYTFLFNEFGEGRHAIR |
| | FQKLLTVEDGVAKEVDDVTVPISMSAQLDD |
| | LLPRDPHELVALYFQDYGAEQHLAGEFGGA |
| | KIQYRRDQLNHLHARRGARDVYLNLSVRVQ |
| | SQSEARGERRPPYAAVFRLVGDNHRAFVHF |
| | DKLSDYLAEHPDDGKLGSEGLLSGLRVMSV |
| | DLGLRTSASISVFRVARKDELKPNSEGRVP |
| | FCFPIEGNENLVAVHERSQLLKLPGETESK |
| | DLRAIREERQRTLRQLRTQLAYLRLLVRCG |
| | SEDVGRRERSWAKLIEQPMDANQMTPDWRE |
| | AFEDELQKLKSLYGICGDREWTEAVYESVR |
| | RVWRHMGKQVRDWRKDVRSGERPKIRGYQK |
| | DVVGGNSIEQIEYLERQYKFLKSWSFFGKV |
| | SGQVIRAEKGSRFAITLREHIDHAKEDRLK |
| | KLADRIIMEALGYVYALDDERGKGKWVAKY |
| | PPCQLILLEELSEYQFNNDRPPSENNQLMQ |
| | WSHRGVFQELLNQAQVHDLLVGTMYAAFSS |
| | RFDARTGAPGIRCRRVPARCAREQNPEPFP |
| | WWLNKFVAEHKLDGCPLRADDLIPTGEGEF |
| | FVSPFSAEEGDFHQIHADLNAAQNLQRRLW |
| | SDFDISQIRLRCDWGEVDGEPVLIPRTTGK |
| | RTADSYGNKVFYTKTGVTYYERERGKKRRK |
| | VFAQEELSEEEAELLVEADEAREKSVVLMR |
| | DPSGIINRGDWTRQKEFWSMVNQRIEGYLV |
| | KQIRSRVRLQESACENTGDI |
| SEQ ID NO: 6 | MAVKSMKVKLRLDNMPEIRAGLWKLHTEVN |
| AaCas12b-Q119W | AGVRYYTEWLSLLRQENLYRRSPNGDGEQE |
| | CYKTAEECKAELLERLRARQVENGHCGPAG |
| | SDDELLQLARQLYELLVPQAIGAKGDAQWI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 7 | MAVKSMKVKLRLDNMPEIRAGLWKLHTEVN |
| AaCas12b-E636R | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIRRERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 8 | MAVKSMKVKLRLDNMPEIRAGLWKLHTEVN |
| AaCas12b-I757R | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSREQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 9 | MAVKSMKVKLRLDNMPEIRAGLWKLHTEVN |
| AaCas12b-E758R | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIRQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 10 | MAVKSMKVKLRLDNMPEIRAGLWKLHTEVN |
| AaCas12b-E761R | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIRYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 11 | MAVKSMKVKLRLDNMPEIRAGLWKLHTEVN |
| AaCas12b-Q854R | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHATIR |
| | FQKLLTVEDGVAKEVDDVTVPISMSAQLDD |
| | LLPRDPHELVALYFQDYGAEQHLAGEFGGA |
| | KIQYRRDQLNHLHARRGARDVYLNLSVRVQ |
| | SQSEARGERRPPYAAVFRLVGDNHRAFVHF |
| | DKLSDYLAEHPDDGKLGSEGLLSGLRVMSV |
| | DLGLRTSASISVFRVARKDELKPNSEGRVP |
| | FCFPIEGNENLVAVHERSQLLKLPGETESK |
| | DLRAIREERQRTLRQLRTQLAYLRLLVRCG |
| | SEDVGRRERSWAKLIEQPMDANQMTPDWRE |
| | AFEDELQKLKSLYGICGDREWTEAVYESVR |
| | RVWRHMGKQVRDWRKDVRSGERPKIRGYQK |
| | DVVGGNSIEQIEYLERQYKFLKSWSFFGKV |
| | SGQVIRAEKGSRFAITLREHIDHAKEDRLK |
| | KLADRIIMEALGYVYALDDERGKGKWVAKY |
| | PPCQLILLEELSEYRFNNDRPPSENNQLMQ |
| | WSHRGVFQELLNQAQVHDLLVGTMYAAFSS |
| | RFDARTGAPGIRCRRVPARCAREQNPEPFP |
| | WWLNKFVAEHKLDGCPLRADDLIPTGEGEF |
| | FVSPFSAEEGDFHQIHADLNAAQNLQRRLW |
| | SDFDISQIRLRCDWGEVDGEPVLIPRTTGK |
| | RTADSYGNKVFYTKTGVTYYERERGKKRRK |
| | VFAQEELSEEEAELLVEADEAREKSVVLMR |
| | DPSGIINRGDWTRQKEFWSMVNQRIEGYLV |
| | KQIRSRVRLQESACENTGDI |
| SEQ ID NO: 12 | MAVKSMKVKLRLDNMPEIRAGLWKLHTEVN |
| AaCas12b-D858R | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNRRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 13 | MAVKSMKVKLRLDNMPEIRAGLWKLHTEVN |
| AaCas12b-N857K | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNKDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 14 | MAVKSMKVKLRLDNMPEIRAGLWKLHTEVN |
| AaCas12b-N865W | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENWQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 15 | MAVKSMKVKLRLDNMPEIRAGLWKLHTEVN |
| AaCas12b-N865Y | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENYQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 16 | MAVKSMKVKLRLDNMPEIRAGLWKLHTEVN |
| AaCas12b-Q866M | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNMLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 17 | MAVKSMKVKLRLDNMPEIRAGLWKLHTEVN |
| AaCas12b-Q869M | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMMW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 18 | MAVKSMKVKLRLDNMPEIRAGLWKLHTEVN |
| AaCas12b-Q1093W | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRWKEFWSMVNQRIEGYLVK |
| | QIRSRVRLQESACENTGDI |
| SEQ ID NO: 19 | MAVKSMKVKLRLDNMPEIRAGLWKLHTEVN |
| AaCas12b-Q1093Y | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRYKEFWSMVNQRIEGYLVK |
| | QIRSRVRLQESACENTGDI |
| SEQ ID NO: 20 | MAVKSMKVKLRLDNMPEIRAGLWKLHTEVN |
| Q866M + Q869M | AGVRYYTEWLSLLRQENLYRRSPNGDGEQE |
| | CYKTAEECKAELLERLRARQVENGHCGPAG |
| | SDDELLQLARQLYELLVPQAIGAKGDAQQI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGEFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIFYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNMLMMW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 21 | MAVKSMKVKLRLDNMPEIRAGLWKLHTEVN |
| AaCas12b- | AGVRYYTEWLSLLRQENLYRRSPNGDGEQE |
| Q119F + E475R | CYKTAEECKAELLERLRARQVENGHCGPAG |
| | SDDELLQLARQLYELLVPQAIGAKGDAQFI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGRFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIEQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 22 | MAVKSMKVKLRLDNMPEIRAGLWKLHTEVN |
| AaCas12b- | AGVRYYTEWLSLLRQENLYRRSPNGDGEQE |
| Q119F + E475R + | CYKTAEECKAELLERLRARQVENGHCGPAG |
| E758R | SDDELLQLARQLYELLVPQAIGAKGDAQFI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGRFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIRQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 79 | MAVKSMKVKLRLDNMPEIRAGLWKLHTEVN |
| AaCas12b- | AGVRYYTEWLSLLRQENLYRRSPNGDGEQE |
| Q119F + E475R + | CYKTAEECKAELLERLRARQVENGHCGPAG |
| E758R + D570A | SDDELLQLARQLYELLVPQAIGAKGDAQFI |
| | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGRFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVA |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIRQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 80 | MAVKSMKVKLRLDNMPEIRAGLWKLHTEVN |
| AaCas12b- | AGVRYYTEWLSLLRQENLYRRSPNGDGEQE |
| Q119F + E475R + | CYKTAEECKAELLERLRARQVENGHCGPAG |
| E758R + D570A | SDDELLQLARQLYELLVPQAIGAKGDAQFI |
| + E848A | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGRFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVA |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIRQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLAELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHADLNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 81 | MAVKSMKVKLRLDNMPEIRAGLWKLHTEVN |
| AaCas12b- | AGVRYYTEWLSLLRQENLYRRSPNGDGEQE |
| Q119F + E475R | CYKTAEECKAELLERLRARQVENGHCGPAG |
| + E758R + D570A | SDDELLQLARQLYELLVPQAIGAKGDAQFI |
| + D977A | ARKFLSPLADKDAVGGLGIAKAGNKPRWVR |
| | MREAGEPGWEEEKAKAEARKSTDRTADVLR |
| | ALADFGLKPLMRVYTDSDMSSVQWKPLRKG |
| | QAVRTWDRDMFQQAIERMMSWESWNQRVGE |
| | AYAKLVEQKSRFEQKNFVGQEHLVQLVNQL |
| | QQDMKEASHGLESKEQTAHYLTGRALRGSD |
| | KVFEKWEKLDPDAPFDLYDTEIKNVQRRNT |
| | RRFGSHDLFAKLAEPKYQALWREDASFLTR |
| | YAVYNSIVRKLNHAKMFATFTLPDATAHPI |
| | WTRFDKLGGNLHQYTFLFNEFGEGRHAIRF |
| | QKLLTVEDGVAKEVDDVTVPISMSAQLDDL |
| | LPRDPHELVALYFQDYGAEQHLAGRFGGAK |
| | IQYRRDQLNHLHARRGARDVYLNLSVRVQS |
| | QSEARGERRPPYAAVFRLVGDNHRAFVHFD |
| | KLSDYLAEHPDDGKLGSEGLLSGLRVMSVA |
| | LGLRTSASISVFRVARKDELKPNSEGRVPF |
| | CFPIEGNENLVAVHERSQLLKLPGETESKD |
| | LRAIREERQRTLRQLRTQLAYLRLLVRCGS |
| | EDVGRRERSWAKLIEQPMDANQMTPDWREA |
| | FEDELQKLKSLYGICGDREWTEAVYESVRR |
| | VWRHMGKQVRDWRKDVRSGERPKIRGYQKD |
| | VVGGNSIRQIEYLERQYKFLKSWSFFGKVS |
| | GQVIRAEKGSRFAITLREHIDHAKEDRLKK |
| | LADRIIMEALGYVYALDDERGKGKWVAKYP |
| | PCQLILLEELSEYQFNNDRPPSENNQLMQW |
| | SHRGVFQELLNQAQVHDLLVGTMYAAFSSR |
| | FDARTGAPGIRCRRVPARCAREQNPEPFPW |
| | WLNKFVAEHKLDGCPLRADDLIPTGEGEFF |
| | VSPFSAEEGDFHQIHAALNAAQNLQRRLWS |
| | DFDISQIRLRCDWGEVDGEPVLIPRTTGKR |
| | TADSYGNKVFYTKTGVTYYERERGKKRRKV |
| | FAQEELSEEEAELLVEADEAREKSVVLMRD |
| | PSGIINRGDWTRQKEFWSMVNQRIEGYLVK |
| | QIRSRVRLQESACENTGDI |
| SEQ ID NO: 23 | UCUAAAGGACAGAAUUUUUCAACGGGUGUG |
| AaCas12b sgRNA | CCAAUGGCCACUUUCCAGGUGGCAAAGCCC |
| scaffold Aa-sg | GUUGAACUUCUCAAAAAGAACGCUCGCUCA |
| | GUGUUCUGACGUCGGAUCACUGAGCGAGCG |
| | AUCUGAGAAGUGGCAC |
| SEQ ID NO: 24 | GGUCUAGAGGACAGAAUUUUUCAACGGGUG |
| sgRNA scaffold V0 | UGCCAAUGGCCACUUUCCAGGUGGCAAAGC |
| (scaffold of | CCGUUGAGCUUCUCAAAUCUGAGAAGUGGC |
| AacCas12b) | AC |
| SEQ ID NO: 25 | GGUCUAGAGGACAGAAGUGCUCAACGGGUG |
| sgRNA scaffold V1 | UGCCAAUGGCCACUUUCCAGGUGGCAAAGC |
| | CCGUUGAGCUUCUCAAAUCUGAGAAGUGGC |
| | AC |
| SEQ ID NO: 26 | GGUCUAGAGGACAGAAGUGCUCAACGGGUG |
| sgRNA scaffold | UGCCAAUGGCCACUUUCCAGGUGGCAAAGC |
| V2.1 | CCGUUGAGCUUCUCAgAAUCcUGAGAAGUG |
| | GCAC |
| SEQ ID NO: 27 | GGUCUAGAGGACAGAAGUGCUCAACGGGUG |
| sgRNA scaffold | UGCCAAUGGCCACUUUCCAGGUGGCAAAGC |
| V2.2 | CCGUUGAGCUUCUCAggAAUCccUGAGAAG |
| | UGGCAC |
| SFO ID NO: 28 | GGUCUAGAGGACAGAAGUGCUCAACGGGUG |
| sgRNA scaffold | UGCCAAUGGCCACUUUCCAGGUGGCAAAGC |
| V2.3 | CCGUUGAGCUUCUCAgggAAUCcccUGAGA |
| | AGUGGCAC |
| SEQ ID NO: 29 | GGUCUAGAGGACAGAAGUGCUCAACGGGUG |
| sgRNA scaffold | UGCCAAUGGCCACUUUCCAGGUGGCAAAGC |
| V2.4 | CCGUUGAGCUUCUCAgggcAAUCgcccUGA |
| | GAAGUGGCAC |
| SEQ ID NO: 30 | GGUCUAGAGGACAGAAGUGCUCAACGGGUG |
| sgRNA scaffold | UGCCAAUGGCCACUUUCCAGGUGGCAAAGC |
| V2.5 | CCGUUGAGCUUCUCAgggccAAUCggcccU |
| | GAGAAGUGGCAC |
| SEQ ID NO: 31 | GGcUCUAGAGGAgCAGAAGUGCUCAACGGG |
| sgRNA scaffold | UGUGCCAAUGGCCACUUUCCAGGUGGCAAA |
| V3.1 | GCCCGUUGAGCUUCUCAAAUCUGAGAAGUG |
| | GCAC |
| SEQ ID NO: 32 | GGccUCUAGAGGAggCAGAAGUGCUCAACG |
| sgRNA scaffold | GGUGUGCCAAUGGCCACUUUCCAGGUGGCA |
| V3.2 | AAGCCCGUUGAGCUUCUCAAAUCUGAGAAG |
| | UGGCAC |
| SEQ ID NO: 33 | GGcccUCUAGAGGAgggCAGAAGUGCUCAA |
| sgRNA scaffold | CGGGUGUGCCAAUGGCCACUUUCCAGGUGG |
| V3.3 | CAAAGCCCGUUGAGCUUCUCAAAUCUGAGA |
| | AGUGGCAC |
| SEQ ID NO: 34 | GGcccgUCUAGAGGAcgggCAGAAGUGCUC |
| sgRNA scaffold | AACGGGUGUGCCAAUGGCCACUUUCCAGGU |
| V3.4 | GGCAAAGCCCGUUGAGCUUCUCAAAUCUGA |
| | GAAGUGGCAC |
| SEQ ID NO: 35 | GGcccggUCUAGAGGAccgggCAGAAGUGC |
| sgRNA scaffold | UCAACGGGUGUGCCAAUGGCCACUUUCCAG |
| V3.5 | GUGGCAAAGCCCGUUGAGCUUCUCAAAUCU |
| | GAGAAGUGGCAC |
| SEQ ID NO: 36 | GGcUCUAGAGGAgCAGAAGUGCUCAACGGG |
| sgRNA scaffold | UGUGCCAAUGGCCACUUUCCAGGUGGCAAA |
| V4.1 | GCCCGUUGAGCUUCUCAgggccAAUCggcc |
| | cUGAGAAGUGGCAC |
| SEQ ID NO: 37 | GGccUCUAGAGGAggCAGAAGUGCUCAACG |
| sgRNA scaffold | GGUGUGCCAAUGGCCACUUUCCAGGUGGCA |
| V4.2 | AAGCCCGUUGAGCUUCUCAgggccAAUCgg |
| | cccUGAGAAGUGGCAC |
| SEQ ID NO: 38 | GGcccUCUAGAGGAgggCAGAAGUGCUCAA |
| sgRNA scaffold | CGGGUGUGCCAAUGGCCACUUUCCAGGUGG |
| V4.3 | CAAAGCCCGUUGAGCUUCUCAgggccAAUC |
| | ggcccUGAGAAGUGGCAC |
| SEQ ID NO: 39 | GGcccgUCUAGAGGAcgggCAGAAGUGCUC |
| sgRNA scaffold | AACGGGUGUGCCAAUGGCCACUUUCCAGGU |
| V4.4 | GGCAAAGCCCGUUGAGCUUCUCAgggccAA |
| | UCggcccUGAGAAGUGGCAC |
| SEQ ID NO: 40 | GGcUCUAGAGGAgCAGAAGUGCUCAACGGG |
| sgRNA scaffold | UGUGCCAAUGGCCACUUUCCAGGUGGCAAA |
| V5.1 | GCCCGUUGAGCUUCUCAggAAUCccUGAGA |
| | AGUGGCAC |
| SEQ ID NO: 41 | GGccUCUAGAGGAggCAGAAGUGCUCAACG |
| sgRNA scaffold | GGUGUGCCAAUGGCCACUUUCCAGGUGGCA |
| V5.2 | AAGCCCGUUGAGCUUCUCAggAAUCccUGA |
| | GAAGUGGCAC |
| SEQ ID NO: 42 | GGcccUCUAGAGGAgggCAGAAGUGCUCAA |
| sgRNA scaffold | CGGGUGUGCCAAUGGCCACUUUCCAGGUGG |
| V5.3 | CAAAGCCCGUUGAGCUUCUCAggAAUCccU |
| | GAGAAGUGGCAC |
| SEQ ID NO: 43 | GGcccgUCUAGAGGAcgggCAGAAGUGCUC |
| sgRNA scaffold | AACGGGUGUGCCAAUGGCCACUUUCCAGGU |
| V5.4 | GGCAAAGCCCGUUGAGCUUCUCAggAAUCc |
| | cUGAGAAGUGGCAC |
| SEQ ID NO: 44 | GGcUCUAGAGGAgCAGAAGUGCUCAACGGG |
| sgRNA scaffold | UGUGCCAAUGGCCACUUUCCAGGUGGCAAA |
| V6.1 | GCCCGUUGAGCUUCUCAgggAAUCcccUGA |
| | GAAGUGGCAC |
| SEQ ID NO: 45 | GGccUCUAGAGGAggCAGAAGUGCUCAACG |
| sgRNA scaffold | GGUGUGCCAAUGGCCACUUUCCAGGUGGCA |
| V6.2 | AAGCCCGUUGAGCUUCUCAgggAAUCcccU |
| | GAGAAGUGGCAC |
| SEQ ID NO: 46 | GGcccUCUAGAGGAgggCAGAAGUGCUCAA |
| sgRNA scaffold | CGGGUGUGCCAAUGGCCACUUUCCAGGUGG |
| V6.3 | CAAAGCCCGUUGAGCUUCUCAgggAAUCcc |
| | cUGAGAAGUGGCAC |
| SEQ ID NO: 47 | GGcccgUCUAGAGGAcgggCAGAAGUGCUC |
| sgRNA scaffold | AACGGGUGUGCCAAUGGCCACUUUCCAGGU |
| V6.4 | GGCAAAGCCCGUUGAGCUUCUCAgggAAUC |
| | cccUGAGAAGUGGCAC |
| SEQ ID NO: 48 | GGcUCUAGAGGAgCAGAAGUGCUCAACGGG |
| sg″ NA scaffold | UGUGCCAAUGGCCACUUUCCAGGUGGCAAA |
| V7.1 | GCCCGUUGAGCUUCUCAgggcAAUCgcccU |
| | GAGAAGUGGCAC |
| SEQ ID NO: 49 | GGccUCUAGAGGAggCAGAAGUGCUCAACG |
| sgRNA scaffold | GGUGUGCCAAUGGCCACUUUCCAGGUGGCA |
| V7.2 | AAGCCCGUUGAGCUUCUCAgggcAAUCgcc |
| | cUGAGAAGUGGCAC |
| SEQ ID NO: 50 | GGcccUCUAGAGGAgggCAGAAGUGCUCAA |
| sgRNA scaffold | CGGGUGUGCCAAUGGCCACUUUCCAGGUGG |
| V7.3 | CAAAGCCCGUUGAGCUUCUCAgggcAAUCg |
| | cccUGAGAAGUGGCAC |
| SEQ ID NO: 51 | GGcccgUCUAGAGGAcgggCAGAAGUGCUC |
| sgRNA scaffold | AACGGGUGUGCCAAUGGCCACUUUCCAGGU |
| V7.4 | GGCAAAGCCCGUUGAGCUUCUCAgggcAAU |
| | CgcccUGAGAAGUGGCAC |
| SEQ ID NO: 52 | GUCUAAAGGACAGAAUUUUUCAACGGGUGU |
| sgRNA scaffold V8 | GCCAAUGGCCACUUUCCAGGUGGCAAAGCC |
| | CGUUGAACUUCAAGCGAAGUGGCAC |
| SEQ ID NO: 53 | GGUCGUCUAUAGGACGGCGAGUUUUUCAAC |
| sgRNA scaffold V9 | GGGUGUGCCAAUGGCCACUUUCCAGGUGGC |
| | AAAGCCCGUUGAGCUUCAAAGAAGUGGCAC |
| SEQ ID NO: 54 | MAVKSIKVKLRLSECPDILAGMWQLHRATN |
| AkCas12b | AGVRYYTEWVSLMRQEILYSRGPDGGQQCY |
| | MTAEDCQRELLRRLRNRQLHNGRQDQPGTD |
| | ADLLAISRRLYEILVLQSIGKRGDAQQIAS |
| | SFLSPLVDPNSKGGRGEAKSGRKPAWQKMR |
| | DQGDPRWVAAREKYEQRKAVDPSKEILNSL |
| | DALGLRPLFAVFTETYRSGVDWKPLGKSQG |
| | VRTWDRDMFQQALERLMSWESWNRRVGEEY |
| | ARLFQQKMKFEQEHFAEQSHLVKLARALEA |
| | DMRAASQGFEAKRGTAHQITRRALRGADRV |
| | FEIWKSIPEEALFSQYDEVIRQVQAEKRRD |
| | FGSHDLFAKLAEPKYQPLWRADETFLTRYA |
| | LYNGVLRDLEKARQFATFTLPDACVNPIWT |
| | RFESSQGSNLHKYEFLFDHLGPGRHAVRFQ |
| | RLLVVESEGAKERDSVVVPVAPSGQLDKLV |
| | LREEEKSSVALHLHDTARPDGFMAEWAGAK |
| | LQYERSTLARKARRDKQGMRSWRRQPSMLM |
| | SAAQMLEDAKQAGDVYLNISVRVKSPSEVR |
| | GQRRPPYAALFRIDDKQRRVTVNYNKLSAY |
| | LEEHPDKQIPGAPGLLSGLRVMSVDLGLRT |
| | SASISVFRVAKKEEVEALGDGRPPHYYPIH |
| | GTDDLVAVHERSHLIQMPGETETKQLRKLR |
| | EERQAVLRPLFAQLALLRLLVRCGAADERI |
| | RTRSWQRLTKQGREFTKRLTPSWREALELE |
| | LTRLEAYCGRVPDDEWSRIVDRTVIALWRR |
| | MGKQVRDWRKQVKSGAKVKVKGYQLDVVGG |
| | NSLAQIDYLEQQYKFLRRWSFFARASGLVV |
| | RADRESHFAVALRQHIENAKRDRLKKLADR |
| | ILMEALGYVYEASGPREGQWTAQHPPCQLI |
| | ILEELSAYRFSDDRPPSENSKLMAWGHRGI |
| | LEELVNQAQVHDVLVGTVYAAFSSRFDART |
| | GAPGVRCRRVPARFVGATVDDSLPLWLTEF |
| | LDKHRLDKNLLRPDDVIPTGEGEFLVSPCG |
| | EEAARVRQVHADINAAQNLQRRLWQNFDIT |
| | ELRLRCDVKMGGEGTVLVPRVNNARAKQLF |
| | GKKVLVSQDGVTFFERSQTGGKPHSEKQTD |
| | LTDKELELIAEADEARAKSVVLFRDPSGHI |
| | GKGHWIRQREFWSLVKQRIESHTAERIRVR |
| | GVGSSLD |
| SEQ ID NO: 55 | MNVAVKSIKVKLMLGHLPEIREGLWHLHEA |
| AmCas12b | VNLGVRYYTEWLALLRQGNLYRRGKDGAQE |
| | CYMTAEQCRQELLVRLRDRQKRNGHTGDPG |
| | TDEELLGVARRLYELLVPQSVGKKGQAQML |
| | ASGFLSPLADPKSEGGKGTSKSGRKPAWMG |
| | MKEAGDSRWVEAKARYEANKAKDPTKQVIA |
| | SLEMYGLRPLFDVFTETYKTIRWMPLGKHQ |
| | GVRAWDRDMFQQSLERLMSWESWNERVGAE |
| | FARLVDRRDRFREKHFTGQEHLVALAQRLE |
| | QEMKEASPGFESKSSQAHRITKRALRGADG |
| | IIDDWLKLSEGEPVDRFDEILRKRQAQNPR |
| | RFGSHDLFLKLAEPVFQPLWREDPSFLSRW |
| | ASYNEVLNKLEDAKQFATFTLPSPCSNPVW |
| | ARFENAEGTNIFKYDFLFDHFGKGRHGVRF |
| | QRMIVMRDGVPTEVEGIVVPIAPSRQLDAL |
| | APNDAASPIDVFVGDPAAPGAFRGQFGGAK |
| | IQYRRSALVRKGRREEKAYLCGFRLPSQRR |
| | TGTPADDAGEVFLNLSLRVESQSEQAGRRN |
| | PPYAAVFHISDQTRRVIVRYGEIERYLAEH |
| | PDTGIPGSRGLTSGLRVMSVDLGLRTSAAI |
| | SVFRVAHRDELTPDAHGRQPFFFPIHGMDH |
| | LVALHERSHLIRLPGETESKKVRSIREQRL |
| | DRLNRLRSQMASLRLLVRTGVLDEQKRDRN |
| | WERLQSSMERGGERMPSDWWDLFQAQVRYL |
| | AQHRDASGEAWGRMVQAAVRTLWRQLAKQV |
| | RDWRKEVRRNADKVKIRGIARDVPGGHSLA |
| | QLDYLERQYRFLRSWSAFSVQAGQVVRAER |
| | DSRFAVALREHIDNGKKDRLKKLADRILME |
| | ALGYVYVTDGRRAGQWQAVYPPCQLVLLEE |
| | LSEYRFSNDRPPSENSQLMVWSHRGVLEEL |
| | IHQAQVHDVLVGTIPAAFSSRFDARTGAPG |
| | IRCRRVPSIPLKDAPSIPIWLSHYLKQTER |
| | DAAALRPGELIPTGDGEFLVTPAGRGASGV |
| | RVVHADINAAHNLQRRLWENFDLSDIRVRC |
| | DRREGKDGTVVLIPRLTNQRVKERYSGVIF |
| | TSEDGVSFTVGDAKTRRRSSASQGEGDDLS |
| | DEEQELLAEADDARERSVVLFRDPSGFVNG |
| | GRWTAQRAFWGMVHNRIETLLAERFSVSGA |
| | AEKVRG |
| SEQ ID NO: 56 | MAIRSIKLKMKTNSGTDSIYLRKALWRTHQ |
| Bs3Cas12b | LINEGIAYYMNLLTLYRQEAIGDKTKEAYQ |
| | AELINIIRNQQRNNGSSEEHGSDQEILALL |
| | RQLYELIIPSSIGESGDANQLGNKFLYPLV |
| | DPNSQSGKGTSNAGRKPRWKRLKEEGNPDW |
| | ELEKKKDEERKAKDPTVKIFDNLNKYGLLP |
| | LFPLFTNIQKDIEWLPLGKRQSVRKWDKDM |
| | FIQAIERLLSWESWNRRVADEYKQLKEKTE |
| | SYYKEHLTGGEEWIEKIRKFEKERNMELEK |
| | NAFAPNDGYFITSRQIRGWDRVYEKWSKLP |
| | ESASPEELWKVVAEQQNKMSEGFGDPKVFS |
| | FLANRENRDIWRGHSERIYHIAAYNGLQKK |
| | LSRTKEQATFTLPDAIEHPLWIRYESPGGT |
| | NLNLFKLEEKQKKNYYVTLSKIIWPSEEKW |
| | IEKENIEIPLAPSIQFNRQIKLKQHVKGKQ |
| | EISFSDYSSRISLDGVLGGSRIQFNRKYIK |
| | NHKELLGEGDIGPVFFNLVVDVAPLQETRN |
| | GRLQSPIGKALKVISSDFSKVIDYKPKELM |
| | DWMNTGSASNSFGVASLLEGMRVMSIDMGQ |
| | RTSASVSIFEVVKELPKDQEQKLFYSINDT |
| | ELFAIHKRSFLLNLPGEVVTKNNKQQRQER |
| | RKKRQFVRSQIRMLANVLRLETKKTPDERK |
| | KAIHKLMEIVQSYDSWTASQKEVWEKELNL |
| | LTNMAAFNDEIWKESLVELHHRIEPYVGQI |
| | VSKWRKGLSEGRKNLAGISMWNIDELEDTR |
| | RLLISWSKRSRTPGEANRIETDEPFGSSLL |
| | QHIQNVKDDRLKQMANLIIMTALGFKYDKE |
| | EKDRYKRWKETYPACQIILFENLNRYLFNL |
| | DRSRRENSRLMKWAHRSIPRTVSMQGEMFG |
| | LQVGDVRSEYSSRFHAKTGAPGIRCHALTE |
| | EDLKAGSNTLKRLIEDGFINESELAYLKKG |
| | DIIPSQGGELFVTLSKRYKKDSDNNELTVI |
| | HADINAAQNLQKRFWQQNSEVYRVPCQLAR |
| | MGEDKLYIPKSQTETIKKYFGKGSFVKNNT |
| | EQEVYKWEKSEKMKIKTDTTFDLQDLDGFE |
| | DISKTIELAQEQQKKYLTMFRDPSGYFFNN |
| | ETWRPQKEYWSIVNNIIKSCLKKKILSNKV |
| | EL |
| SEQ ID NO: 57 | MAIRSIKLKLKTHTGPEAQNLRKGIWRTHR |
| BsCas12b | LLNEGVAYYMKMLLLFRQESTGERPKEELQ |
| | EELICHIREQQQRNQADKNTQALPLDKALE |
| | ALRQLYELLVPSSVGQSGDAQIISRKFLSP |
| | LVDPNSEGGKGTSKAGAKPTWQKKKEANDP |
| | TWEQDYEKWKKRREEDPTASVITTLEEYGI |
| | RPIFPLYTNTVTDIAWLPLQSNQFVRTWDR |
| | DMLQQAIERLLSWESWNKRVQEEYAKLKEK |
| | MAQLNEQLEGGQEWISLLEQYEENRERELR |
| | ENMTAANDKYRITKRQMKGWNELYELWSTF |
| | PASASHEQYKEALKRVQQRLRGRFGDAHFF |
| | QYLMEEKNRLIWKGNPQRIHYFVARNELTK |
| | RLEEAKQSATMTLPNARKHPLWVRFDARGG |
| | NLQDYYLTAEADKPRSRRFVTFSQLIWPSE |
| | SGWMEKKDVEVELALSRQFYQQVKLLKNDK |
| | GKQKIEFKDKGSGSTFNGHLGGAKLQLERG |
| | DLEKEEKNFEDGEIGSVYLNVVIDFEPLQE |
| | VKNGRVQAPYGQVLQLIRRPNEFPKVTTYK |
| | SEQLVEWIKASPQHSAGVESLASGFRVMSI |
| | DLGLRAAAATSIFSVEESSDKNAADFSYWI |
| | EGTPLVAVHQRSYMLRLPGEQVEKQVMEKR |
| | DERFQLHQRVKFQIRVLAQIMRMANKQYGD |
| | RWDELDSLKQAVEQKKSPLDQTDRTFWEGI |
| | VCDLTKVLPRNEADWEQAVVQIHRKAEEYV |
| | GKAVQAWRKRFAADERKGIAGLSMWNIEEL |
| | EGLRKLLISWSRRTRNPQEVNRFERGHTSH |
| | QRLLTHIQNVKEDRLKQLSHAIVMTALGYV |
| | YDERKQEWCAEYPACQVILFENLSQYRSNL |
| | DRSTKENSTLMKWAHRSIPKYVHMQAEPYG |
| | IQIGDVRAEYSSRFYAKTGTPGIRCKKVRG |
| | QDLQGRRFENLQKRLVNEQFLTEEQVKQLR |
| | PGDIVPDDSGELFMTLTDGSGSKEVVFLQA |
| | DINAAHNLQKRFWQRYNELFKVSCRVIVRD |
| | EEEYLVPKTKSVQAKLGKGLFVKKSDTAWK |
| | DVYVWDSQAKLKGKTTFTEESESPEQLEDF |
| | QEIIEEAEEAKGTYRTLFRDPSGVFFPESV |
| | WYPQKDFWGEVKRKLYGKLRERFLTKAR |
| SEQ ID NO: 58 | MSIRSFKLKIKTKSGVNAEELRRGLWRTHQ |
| LsCas12b | LINDGIAYYMNWLVLLRQEDLFIRNEETNE |
| | IEKRSKEEIQGELLERVHKQQQRNQWSGEV |
| | DDQTLLQTLRHLYEEIVPSVIGKSGNASLK |
| | ARFFLGPLVDPNNKTTKDVSKSGPTPKWKK |
| | MKDAGDPNWVQEYEKYMAERQTLVRLEEMG |
| | LIPLFPMYTDEVGDIHWLPQASGYTRTWDR |
| | DMFQQAIERLLSWESWNRRVRERRAQFEKK |
| | THDFASRFSESDVQWMNKLREYEAQQEKSL |
| | EENAFAPNEPYALTKKALRGWERVYHSWMR |
| | LDSAASEEAYWQEVATCQTAMRGEFGDPAI |
| | YQFLAQKENHDIWRGYPERVIDFAELNHLQ |
| | RELRRAKEDATFTLPDSVDHPLWVRYEAPG |
| | GTNIHGYDLVQDTKRNLTLILDKFILPDEN |
| | GSWHEVKKVPFSLAKSKQFHRQVWLQEEQK |
| | QKKREVVFYDYSTNLPHLGTLAGAKLQWDR |
| | NFLNKRTQQQIEETGEIGKVFFNISVDVRP |
| | AVEVKNGRLQNGLGKALTVLTHPDGTKIVT |
| | GWKAEQLEKWVGESGRVSSLGLDSLSEGLR |
| | VMSIDLGQRTSATVSVFEITKEAPDNPYKF |
| | FYQLEGTELFAVHQRSFLLALPGENPPQKI |
| | KQMREIRWKERNRIKQQVDQLSAILRLHKK |
| | VNEDERIQAIDKLLQKVASWQLNEEIATAW |
| | NQALSQLYSKAKENDLQWNQAIKNAHHQLE |
| | PVVGKQISLWRKDLSTGRQGIAGLSLWSIE |
| | ELEATKKLLTRWSKRSREPGVVKRIERFET |
| | FAKQIQHHINQVKENRLKQLANLIVMTALG |
| | YKYDQEQKKWIEVYPACQVVLFENLRSYRF |
| | SYERSRRENKKLMEWSHRSIPKLVQMQGEL |
| | FGLQVADVYAAYSSRYHGRTGAPGIRCHAL |
| | TEADLRNETNIIHELIEAGFIKEEHRPYLQ |
| | QGDLVPWSGGELFATLQKPYDNPRILTLHA |
| | DINAAQNIQKRFWHPSMWFRVNCESVMEGE |
| | IVTYVPKNKTVHKKQGKTFRFVKVEGSDVY |
| | EWAKWSKNRNKNTFSSITERKPPSSMILFR |
| | DPSGTFFKEQEWVEQKTFWGKVQSMIQAYM |
| | KKTIVQRMEE |
| SEQ ID NO: 59 | MATRSFILKIEPNEEVKKGLWKTHEVLNHG |
| BhCas12b | IAYYMNILKLIRQEAIYEHHEQDPKNPKKV |
| | SKAEIQAELWDFVLKMQKCNSFTHEVDKDE |
| | VFNILRELYEELVPSSVEKKGEANQLSNKF |
| | LYPLVDPNSQSGKGTASSGRKPRWYNLKIA |
| | GDPSWEEEKKKWEEDKKKDPLAKILGKLAE |
| | YGLIPLFIPYTDSNEPIVKEIKWMEKSRNQ |
| | SVRRLDKDMFIQALERFLSWESWNLKVKEE |
| | YEKVEKEYKTLEERIKEDIQALKALEQYEK |
| | ERQEQLLRDTLNTNEYRLSKRGLRGWREII |
| | QKWLKMDENEPSEKYLEVFKDYQRKHPREA |
| | GDYSVYEFLSKKENHFIWRNHPEYPYLYAT |
| | FCEIDKKKKDAKQQATFTLADPINHPLWVR |
| | FEERSGSNLNKYRILTEQLHTEKLKKKLTV |
| | QLDRLIYPTESGGWEEKGKVDIVLLPSRQF |
| | YNQIFLDIEEKGKHAFTYKDESIKFPLKGT |
| | LGGARVQFDRDHLRRYPHKVESGNVGRIYF |
| | NMTVNIEPTESPVSKSLKIHRDDFPKVVNF |
| | KPKELTEWIKDSKGKKLKSGIESLEIGLRV |
| | MSIDLGQRQAAAASI |
| SEQ ID NO: 60 | FEVVDQKPDIEGKLFFPIKGTELYAVHRAS |
| SbCas12b | FNIKLPGETLVKSREVLRKAREDNLKLMNQ |
| | KLNFLRNVLHFQQFEDITEREKRVTKWISR |
| | QENSDVPLVYQDELIQIRELMYKPYKDWVA |
| | FLKQLHKRLEVEIGKEVKHWRKSLSDGRKG |
| | LYGISLKNIDEIDRTRKFLLRWSLRPTEPG |
| | EVRRLEPGQRFAIDQLNHLNALKEDRLKKM |
| | ANTIIMHALGYCYDVRKKKWQAKNPACQII |
| | LFEDLSNYNPYEERSRFENSKLMKWSRREI |
| | PRQVALQGEIYGLQVGEVGAQFSSRFHAKT |
| | GSPGIRCSVVTKEKLQDNRFFKNLQREGRL |
| | TLDKIAVLKEGDLYPDKGGEKFISLSKDRK |
| | CVTTHADINAAQNLQKRFWTRTHGFYKVYC |
| | KAYQVDGQTVYIPESKDQKQKIIEEFGEGY |
| | FILKDGVYEWVNAGKLKIKKGSSKQSSSEL |
| | VDSDILKDSFDLASELKGEKLMLYRDPSGN |
| | VFPSDKWMAAGVFFGKLERILISKLTNQYS |
| | ISTIEDDSSKQSMMSFTISYPFKLIIKNKD |
| | EAKALLDTHQYMNEGVKYYLEKLLMFRQEK |
| | IFIGEDETGKRIYIEETEYKKQIEEFYLIK |
| | KTELGRNLTLTLDEFKTLMRELYICLVSSS |
| | MENKKGFPNAQQASLNIFSPLFDAESKGYI |
| | LKEENNNISLIHKDYGKILLKRLRDNNLIP |
| | IFTKFTDIKKITAKLSPTALDRMIFAQAIE |
| | KLLSYESWCKLMIKERFDKEVKIKELENKC |
| | ENKQERDKIFEILEKYEEERQKTFEQDSGF |
| | AKKGKFYITGRMLKGFDEIKEKWLKEKDRS |
| | EQNLINILNKYQTDNSKLVGDRNLFEFIIK |
| | LENQCLWNGDIDYLKIKRDINKNQIWLDRP |
| | EMPRFTMPDFKKHPLWYRYEDPSNSNFRNY |
| | KIEVVKDENYITIPLITERNNEYFEENYTF |
| | NLAKLKKLSENITFIPKSKNKEFEFIDSND |
| | EEEDKKDQKKSKQYIKYCDTAKNTSYGKSG |
| | GIRLYFNRNELENYKDGKKMDSYTVFTLSI |
| | RDYKSLFAKEKLQPQIFNTVDNKITSLKIQ |
| | KKFGNEEQTNFLSYFTQNQITKKDWMDEKT |
| | FQNVKELNEGIRVLSVDLGQRFFAAVSCFE |
| | IMSEIDNNKLFFNLNDQNHKIIRINDKNYY |
| | AKHIYSKTIKLSGEDDDLYKERKINKNYKL |
| | SYQERKNKIGIFTRQINKLNQLLKIIRNDE |
| | IDKEKFKELIETTKRYVKNTYNDGIIDWNN |
| | VDNKILSYENKEDVINLHKELDKKLEIDFK |
| | EFIRECRKPIFRSGGLSMQRIDFLEKLNKL |
| | KRKWVARTQKSAESIVLTPKFGYKLKEHIN |
| | ELKDNRVKQGVNYILMTALGYIKDNEIKND |
| | SKKKQKEDWVKKNRACQIILMEKLTEYTFA |
| | EDRPREENSKLRMWSHRQIFNFLQQKASLW |
| | GILVGDVFAPYTSKCLSDNNAPGIRCHQVT |
| | KKDLIDNSWFLKIVVKDDAFCDLIEINKEN |
| | VKNKSIKINDILPLRGGELFASIKDGKLHI |
| | VQADINASRNIAKRFLSQINPFRVVLKKDK |
| | DETFHLKNEPNYLKNYYSILNFVPTNEELT |
| | FFKVEENKDIKPTKRIKMDKHEKESTDEGD |
| | DYSKNQIALFRDDSGIFFDKSLWVDGKIFW |
| | SVVKNKMTKLLRERNNKKNGSK |
| SEQ ID NO: 61 | PKKKRKVPG |
| NLS | |
| SEQ ID NO: 62 | ASPKKKRKV |
| NLS | |
| SEQ ID NO: 82 | PKKKRKV |
| NLS | |
| SEQ ID NO: 63 | CTCTTCTGGGCTCCCTACAA |
| CCR5-11 target site | |
| SEQ ID NO: 64 | AACCACTAGCACTAGCCTTG |
| CD34-7 target site | |
| SEQ ID NO: 65 | TACAGGAGGCAATAACAGAT |
| RNF2-1 target site | |
| SEQ ID NO: 66 | AGGCCAAAGAATTCCTGGAA |
| CCR5-3 target site | |
| SEQ ID NO: 67 | GGCACATTAATTCACTGTGT |
| RNF2-5 target site | |
| SEQ ID NO: 68 | CAGGTGACAGGCTAGGCTTC |
| CD34-1 target site | |
| SEQ ID NO: 72 | MNNSQGRVTFEDVTVNFTQGEWQRLNPEQR |
| Krüppel | NLYRDVMLENYSNLVSVGQGETTKPDVILR |
| associated | LEQGKEPWLEEEEVLGSGRAEKNGDIGGQI |
| box (KRAB) | WKPKDVKESL |
| domain | |

1. An engineered Cas12b nuclease, comprising one, two, or three types of mutations with respect to a reference Cas12b nuclease, wherein the mutations comprise:
(1) substitution of one or more amino acid residues in the reference Cas12b nuclease that interact with a protospacer adjacent motif (PAM) with a positively charged amino acid residue; and/or
(2) substitution of one or more amino acid residues in the reference Cas12b nuclease that are involved in opening DNA double strands with an amino acid residue having an aromatic ring; and/or
(3) substitution of one or more amino acid residues in the RuvC domain of the reference Cas12b nuclease that interact with a single-stranded DNA substrate with a positively charged amino acid residue or a hydrophobic amino acid residue.

2. (canceled)

3. The engineered Cas12b nuclease of claim 1, wherein the reference Cas12b nuclease comprises the amino acid sequence of SEQ ID NO: 1.

4-5. (canceled)

6. The engineered Cas12b nuclease of claim 1, wherein the engineered Cas12b nuclease comprises substitution of one or more amino acid residues in the reference Cas12b nuclease that interact with PAM with a positively charged amino acid residue, wherein the one or more amino acid residues that interact with PAM are in one or more of the following positions: 116, 123, 130, 132, 144, 145, 153, 173, 222, 395, 400, and 475; and wherein the amino acid residue numbering is according to SEQ ID NO: 1.

7-12. (canceled)

13. The engineered Cas12b nuclease of claim 1, wherein the engineered Cas12b nuclease comprises substitution of one or more amino acid residues in the reference Cas12b nuclease that are involved in opening the DNA double strands with an amino acid residue having an aromatic ring, wherein the one or more amino acid residues that are involved in opening the DNA double strands are in one or more of the following positions: 118 and 119; and wherein the amino acid residue numbering is according to SEQ ID NO: 1.

14-18. (canceled)

19. The engineered Cas12b nuclease of claim 1, wherein the engineered Cas12b nuclease comprises substitution of one or more amino acid residues in the reference Cas12b nuclease that are in the RuvC domain and interact with the single-stranded DNA substrate with a positively charged amino acid residue or a hydrophobic amino acid residue, wherein the one or more amino acid residues that are in the RuvC domain and interact with the single-stranded DNA substrate are in one or more of the following positions: 300, 301, 304, 329, 636, 639, 647, 682, 757, 758, 761, 764, 768, 852, 854, 856, 857, 858, 860, 862, 863, 865, 866, 867, 869, 938, 956, 957, 958, 994, 1093, and 1097; and wherein the amino acid residue numbering is according to SEQ ID NO: 1.

20-27. (canceled)

28. The engineered Cas12b nuclease of claim 1, wherein the engineered Cas12b nuclease comprises any one of the following substitutions or combinations thereof: (1) D116R; (2) E475R; (3) Q119F+E475R; (4) Q119F+E475R+E758R; (5) Q119Y; (6) Q119F; (7) Q119W; (8) I757R; (9) E758R; (10) E761R; (11) K768R; (12) I757R+E758R; (13) I757R+E761R; (14) I757R+K768R; (15) E758R+E761R; (16) E758R+K768R; (17) E761R+K768R; (18) I757R+E758R+E761R; (19) I757R+E758R+K768R; (20) I757R+E761R+K768R; (21) E758R+E761R+K768R; (22) I757R+E758R+E761R+K768R; (23) Q866M; (24) Q869M; (25) Q866M+Q869M; (26) E636R; (27) Q854R; (28) N857K; (29) N865W; (30) N865Y; (31) Q1093W; (32) Q1093Y; and (33) D858R; and wherein the amino acid residue numbering is according to SEQ ID NO: 1.

29. The engineered Cas12b nuclease of claim 1, wherein the engineered Cas12b nuclease comprises any one of the following substitutions or combinations thereof: (1) Q866M+Q869M; (2) Q119F+E475R; and (3) Q119F+E475R+E758R; and wherein the amino acid residue numbering is according to SEQ ID NO: 1.

30. The engineered Cas12b nuclease of claim 1, wherein the engineered Cas12b nuclease comprises an amino acid sequence of any one of SEQ ID NOs: 20-22.

31. (canceled)

32. The engineered Cas12b nuclease of claim 1, further comprising one or more mutations in the reference Cas12b nuclease that increase flexibility of a flexible region comprising amino acid residues 855-859, wherein the amino acid residue numbering is according to SEQ ID NO: 1, and wherein the one or more mutations that increase flexibility comprises N856G.

33. An engineered Cas12b effector protein comprising the engineered Cas12b nuclease of claim 1 or a functional derivative thereof.

34-38. (canceled)

39. The engineered Cas12b effector protein of claim 33, wherein the engineered Cas12b effector protein further comprises a functional domain fused to the engineered Cas12b nuclease or functional derivative thereof.

40-42. (canceled)

43. A single guide RNA (sgRNA) comprising the sequence of any one of SEQ ID NOs: 25-53.

44. An engineered CRISPR-Cas12b system, comprising:
(I)(a) the engineered Cas12b nuclease of claim 1, or an engineered Cas12b effector protein comprising thereof, or a nucleic acid encoding thereof; and
(b) a guide RNA (gRNA) comprising a guide sequence complementary to a target sequence of a target nucleic acid, or a nucleic acid encoding the gRNA,
wherein the engineered Cas12b nuclease or the engineered Cas12b effector protein and the gRNA are capable of forming a CRISPR complex that specifically binds to the target nucleic acid and inducing a modification of the target nucleic acid; or
(II) (a) a Cas12b nuclease or a Cas12b effector protein comprising the amino acid sequence of any of SEQ ID NOs: 1-22 and 79-81, or a nucleic acid encoding thereof; and
(b) a gRNA comprising a guide sequence complementary to a target sequence of a target nucleic acid, or a nucleic acid encoding the gRNA, wherein the gRNA comprises an engineered scaffold comprising the sequence of any of SEQ ID NOs: 25-53;
wherein the Cas12b nuclease or the Cas12b effector protein and the gRNA are capable of forming a CRISPR complex that specifically binds to the target nucleic acid and inducing a modification of the target nucleic acid.

45-55. (canceled)

56. A method of detecting a target nucleic acid in a sample, comprising:
(a) contacting the sample with the engineered CRISPR-Cas12b system of claim 44 and a labeled detector nucleic acid, wherein the gRNA comprises a guide sequence complementary to a target sequence of the target nucleic acid, and wherein the labeled detector nucleic acid is single-stranded and does not hybridize with the guide sequence of the gRNA; and
(b) measuring a detectable signal produced by cleavage of the labeled detector nucleic acid by the engineered Cas12b nuclease or effector protein thereof, or by the Cas12b nuclease or effector protein thereof, thereby detecting the target nucleic acid.

57. A method of modifying a target nucleic acid comprising a target sequence, comprising contacting the target nucleic acid with the engineered CRISPR-Cas12b system of claim 44.

58-67. (canceled)

68. A method of treating a disease or a condition associated with a target nucleic acid in a cell of an individual, comprising modifying the target nucleic acid in the cell of the individual using the engineered CRISPR-Cas12b system of claim 44, thereby treating the disease or the condition.

69. (canceled)

70. An engineered cell comprising a modified target nucleic acid, wherein the target nucleic acid is modified using the method of claim 57.

71. An engineered non-human animal comprising one or more engineered cells of claim 70.


SEQUENCE LISTING (82 sequences)

SEQ ID NO: 1 - protein, 1129 aa, Alicyclobacillus acidiphilus
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 2 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGRAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 3 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGRFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 4 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQYIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 5 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQFIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 6 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQWIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 7 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIRRERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 8 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSREQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 9 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIRQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 10 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIRYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 11 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYRFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 12 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNRRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 13 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNKDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 14 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENWQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 15 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENYQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 16 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNMLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 17 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMMWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 18 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRWKEFWSMVNQRIEGYLVKQIRSRVRLQESACENTGDI

SEQ ID NO: 19 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRYKEFWSMVNQRIEGYLVKQIRSRVRLQESACENTGDI

SEQ ID NO: 20 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNMLMMWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 21 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQFIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGRFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 22 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQFIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGRFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIRQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 23 - RNA, 136 nt, synthetic construct
  source 1..136: mol_type = other RNA
tctaaaggacagaatttttcaacgggtgtgccaatggccactttccaggtggcaaagcccgttgaacttctcaaaaagaacgctcgctcagtgttctgacgtcggatcactgagcgagcgatctgagaagtggcac

SEQ ID NO: 24 - RNA, 92 nt, synthetic construct
  source 1..92: mol_type = other RNA
ggtctagaggacagaatttttcaacgggtgtgccaatggccactttccaggtggcaaagcccgttgagcttctcaaatctgagaagtggcac

SEQ ID NO: 25 - RNA, 92 nt, synthetic construct
  source 1..92: mol_type = other RNA
ggtctagaggacagaagtgctcaacgggtgtgccaatggccactttccaggtggcaaagcccgttgagcttctcaaatctgagaagtggcac

SEQ ID NO: 26 - RNA, 94 nt, synthetic construct
  source 1..94: mol_type = other RNA
ggtctagaggacagaagtgctcaacgggtgtgccaatggccactttccaggtggcaaagcccgttgagcttctcagaatcctgagaagtggcac

SEQ ID NO: 27 - RNA, 96 nt, synthetic construct
  source 1..96: mol_type = other RNA
ggtctagaggacagaagtgctcaacgggtgtgccaatggccactttccaggtggcaaagcccgttgagcttctcaggaatccctgagaagtggcac

SEQ ID NO: 28 - RNA, 98 nt, synthetic construct
  source 1..98: mol_type = other RNA
ggtctagaggacagaagtgctcaacgggtgtgccaatggccactttccaggtggcaaagcccgttgagcttctcagggaatcccctgagaagtggcac

SEQ ID NO: 29 - RNA, 100 nt, synthetic construct
  source 1..100: mol_type = other RNA
ggtctagaggacagaagtgctcaacgggtgtgccaatggccactttccaggtggcaaagcccgttgagcttctcagggcaatcgccctgagaagtggcac

SEQ ID NO: 30 - RNA, 102 nt, synthetic construct
  source 1..102: mol_type = other RNA
ggtctagaggacagaagtgctcaacgggtgtgccaatggccactttccaggtggcaaagcccgttgagcttctcagggccaatcggccctgagaagtggcac

SEQ ID NO: 31 - RNA, 94 nt, synthetic construct
  source 1..94: mol_type = other RNA
ggctctagaggagcagaagtgctcaacgggtgtgccaatggccactttccaggtggcaaagcccgttgagcttctcaaatctgagaagtggcac

SEQ ID NO: 32 - RNA, 96 nt, synthetic construct
  source 1..96: mol_type = other RNA
ggcctctagaggaggcagaagtgctcaacgggtgtgccaatggccactttccaggtggcaaagcccgttgagcttctcaaatctgagaagtggcac

SEQ ID NO: 33 - RNA, 98 nt, synthetic construct
  source 1..98: mol_type = other RNA
ggccctctagaggagggcagaagtgctcaacgggtgtgccaatggccactttccaggtggcaaagcccgttgagcttctcaaatctgagaagtggcac

SEQ ID NO: 34 - RNA, 100 nt, synthetic construct
  source 1..100: mol_type = other RNA
ggcccgtctagaggacgggcagaagtgctcaacgggtgtgccaatggccactttccaggtggcaaagcccgttgagcttctcaaatctgagaagtggcac

SEQ ID NO: 35 - RNA, 102 nt, synthetic construct
  source 1..102: mol_type = other RNA
ggcccggtctagaggaccgggcagaagtgctcaacgggtgtgccaatggccactttccaggtggcaaagcccgttgagcttctcaaatctgagaagtggcac

SEQ ID NO: 36 - RNA, 104 nt, synthetic construct
  source 1..104: mol_type = other RNA
ggctctagaggagcagaagtgctcaacgggtgtgccaatggccactttccaggtggcaaagcccgttgagcttctcagggccaatcggccctgagaagtggcac

SEQ ID NO: 37 - RNA, 106 nt, synthetic construct
  source 1..106: mol_type = other RNA
ggcctctagaggaggcagaagtgctcaacgggtgtgccaatggccactttccaggtggcaaagcccgttgagcttctcagggccaatcggccctgagaagtggcac

SEQ ID NO: 38 - RNA, 108 nt, synthetic construct
  source 1..108: mol_type = other RNA
ggccctctagaggagggcagaagtgctcaacgggtgtgccaatggccactttccaggtggcaaagcccgttgagcttctcagggccaatcggccctgagaagtggcac

SEQ ID NO: 39 - RNA, 110 nt, synthetic construct
  source 1..110: mol_type = other RNA
ggcccgtctagaggacgggcagaagtgctcaacgggtgtgccaatggccactttccaggtggcaaagcccgttgagcttctcagggccaatcggccctgagaagtggcac

SEQ ID NO: 40 - RNA, 98 nt, synthetic construct
  source 1..98: mol_type = other RNA
ggctctagaggagcagaagtgctcaacgggtgtgccaatggccactttccaggtggcaaagcccgttgagcttctcaggaatccctgagaagtggcac

SEQ ID NO: 41 - RNA, 100 nt, synthetic construct
  source 1..100: mol_type = other RNA
ggcctctagaggaggcagaagtgctcaacgggtgtgccaatggccactttccaggtggcaaagcccgttgagcttctcaggaatccctgagaagtggcac

SEQ ID NO: 42 - RNA, 102 nt, synthetic construct
  source 1..102: mol_type = other RNA
ggccctctagaggagggcagaagtgctcaacgggtgtgccaatggccactttccaggtggcaaagcccgttgagcttctcaggaatccctgagaagtggcac

SEQ ID NO: 43 - RNA, 104 nt, synthetic construct
  source 1..104: mol_type = other RNA
ggcccgtctagaggacgggcagaagtgctcaacgggtgtgccaatggccactttccaggtggcaaagcccgttgagcttctcaggaatccctgagaagtggcac

SEQ ID NO: 44 - RNA, 100 nt, synthetic construct
  source 1..100: mol_type = other RNA
ggctctagaggagcagaagtgctcaacgggtgtgccaatggccactttccaggtggcaaagcccgttgagcttctcagggaatcccctgagaagtggcac

SEQ ID NO: 45 - RNA, 102 nt, synthetic construct
  source 1..102: mol_type = other RNA
ggcctctagaggaggcagaagtgctcaacgggtgtgccaatggccactttccaggtggcaaagcccgttgagcttctcagggaatcccctgagaagtggcac

SEQ ID NO: 46 - RNA, 104 nt, synthetic construct
  source 1..104: mol_type = other RNA
ggccctctagaggagggcagaagtgctcaacgggtgtgccaatggccactttccaggtggcaaagcccgttgagcttctcagggaatcccctgagaagtggcac

SEQ ID NO: 47 - RNA, 106 nt, synthetic construct
  source 1..106: mol_type = other RNA
ggcccgtctagaggacgggcagaagtgctcaacgggtgtgccaatggccactttccaggtggcaaagcccgttgagcttctcagggaatcccctgagaagtggcac

SEQ ID NO: 48 - RNA, 102 nt, synthetic construct
  source 1..102: mol_type = other RNA
ggctctagaggagcagaagtgctcaacgggtgtgccaatggccactttccaggtggcaaagcccgttgagcttctcagggcaatcgccctgagaagtggcac

SEQ ID NO: 49 - RNA, 104 nt, synthetic construct
  source 1..104: mol_type = other RNA
ggcctctagaggaggcagaagtgctcaacgggtgtgccaatggccactttccaggtggcaaagcccgttgagcttctcagggcaatcgccctgagaagtggcac

SEQ ID NO: 50 - RNA, 106 nt, synthetic construct
  source 1..106: mol_type = other RNA
ggccctctagaggagggcagaagtgctcaacgggtgtgccaatggccactttccaggtggcaaagcccgttgagcttctcagggcaatcgccctgagaagtggcac

SEQ ID NO: 51 - RNA, 108 nt, synthetic construct
  source 1..108: mol_type = other RNA
ggcccgtctagaggacgggcagaagtgctcaacgggtgtgccaatggccactttccaggtggcaaagcccgttgagcttctcagggcaatcgccctgagaagtggcac

SEQ ID NO: 52 - RNA, 85 nt, synthetic construct
  source 1..85: mol_type = other RNA
gtctaaaggacagaatttttcaacgggtgtgccaatggccactttccaggtggcaaagcccgttgaacttcaagcgaagtggcac

SEQ ID NO: 53 - RNA, 90 nt, synthetic construct
  source 1..90: mol_type = other RNA
ggtcgtctataggacggcgagtttttcaacgggtgtgccaatggccactttccaggtggcaaagcccgttgagcttcaaagaagtggcac

SEQ ID NO: 54 - protein, 1147 aa, Alicyclobacillus kakegawensis
MAVKSIKVKLRLSECPDILAGMWQLHRATNAGVRYYTEWVSLMRQEILYSRGPDGGQQCYMTAEDCQRELLRRLRNRQLHNGRQDQPGTDADLLAISRRLYEILVLQSIGKRGDAQQIASSFLSPLVDPNSKGGRGEAKSGRKPAWQKMRDQGDPRWVAAREKYEQRKAVDPSKEILNSLDALGLRPLFAVFTETYRSGVDWKPLGKSQGVRTWDRDMFQQALERLMSWESWNRRVGEEYARLFQQKMKFEQEHFAEQSHLVKLARALEADMRAASQGFEAKRGTAHQITRRALRGADRVFEIWKSIPEEALFSQYDEVIRQVQAEKRRDFGSHDLFAKLAEPKYQPLWRADETFLTRYALYNGVLRDLEKARQFATFTLPDACVNPIWTRFESSQGSNLHKYEFLFDHLGPGRHAVRFQRLLVVESEGAKERDSVVVPVAPSGQLDKLVLREEEKSSVALHLHDTARPDGFMAEWAGAKLQYERSTLARKARRDKQGMRSWRRQPSMLMSAAQMLEDAKQAGDVYLNISVRVKSPSEVRGQRRPPYAALFRIDDKQRRVTVNYNKLSAYLEEHPDKQIPGAPGLLSGLRVMSVDLGLRTSASISVFRVAKKEEVEALGDGRPPHYYPIHGTDDLVAVHERSHLIQMPGETETKQLRKLREERQAVLRPLFAQLALLRLLVRCGAADERIRTRSWQRLTKQGREFTKRLTPSWREALELELTRLEAYCGRVPDDEWSRIVDRTVIALWRRMGKQVRDWRKQVKSGAKVKVKGYQLDVVGGNSLAQIDYLEQQYKFLRRWSFFARASGLVVRADRESHFAVALRQHIENAKRDRLKKLADRILMEALGYVYEASGPREGQWTAQHPPCQLIILEELSAYRFSDDRPPSENSKLMAWGHRGILEELVNQAQVHDVLVGTVYAAFSSRFDARTGAPGVRCRRVPARFVGATVDDSLPLWLTEFLDKHRLDKNLLRPDDVIPTGEGEFLVSPCGEEAARVRQVHADINAAQNLQRRLWQNFDITELRLRCDVKMGGEGTVLVPRVNNARAKQLFGKKVLVSQDGVTFFERSQTGGKPHSEKQTDLTDKELELIAEADEARAKSVVLFRDPSGHIGKGHWIRQREFWSLVKQRIESHTAERIRVRGVGSSLD

SEQ ID NO: 55 - protein, 1146 aa, Alicyclobacillus macrosporangiidus
MNVAVKSIKVKLMLGHLPEIREGLWHLHEAVNLGVRYYTEWLALLRQGNLYRRGKDGAQECYMTAEQCRQELLVRLRDRQKRNGHTGDPGTDEELLGVARRLYELLVPQSVGKKGQAQMLASGFLSPLADPKSEGGKGTSKSGRKPAWMGMKEAGDSRWVEAKARYEANKAKDPTKQVIASLEMYGLRPLFDVFTETYKTIRWMPLGKHQGVRAWDRDMFQQSLERLMSWESWNERVGAEFARLVDRRDRFREKHFTGQEHLVALAQRLEQEMKEASPGFESKSSQAHRITKRALRGADGIIDDWLKLSEGEPVDRFDEILRKRQAQNPRRFGSHDLFLKLAEPVFQPLWREDPSFLSRWASYNEVLNKLEDAKQFATFTLPSPCSNPVWARFENAEGTNIFKYDFLFDHFGKGRHGVRFQRMIVMRDGVPTEVEGIVVPIAPSRQLDALAPNDAASPIDVFVGDPAAPGAFRGQFGGAKIQYRRSALVRKGRREEKAYLCGFRLPSQRRTGTPADDAGEVFLNLSLRVESQSEQAGRRNPPYAAVFHISDQTRRVIVRYGEIERYLAEHPDTGIPGSRGLTSGLRVMSVDLGLRTSAAISVFRVAHRDELTPDAHGRQPFFFPIHGMDHLVALHERSHLIRLPGETESKKVRSIREQRLDRLNRLRSQMASLRLLVRTGVLDEQKRDRNWERLQSSMERGGERMPSDWWDLFQAQVRYLAQHRDASGEAWGRMVQAAVRTLWRQLAKQVRDWRKEVRRNADKVKIRGIARDVPGGHSLAQLDYLERQYRFLRSWSAFSVQAGQVVRAERDSRFAVALREHIDNGKKDRLKKLADRILMEALGYVYVTDGRRAGQWQAVYPPCQLVLLEELSEYRFSNDRPPSENSQLMVWSHRGVLEELIHQAQVHDVLVGTIPAAFSSRFDARTGAPGIRCRRVPSIPLKDAPSIPIWLSHYLKQTERDAAALRPGELIPTGDGEFLVTPAGRGASGVRVVHADINAAHNLQRRLWENFDLSDIRVRCDRREGKDGTVVLIPRLTNQRVKERYSGVIFTSEDGVSFTVGDAKTRRRSSASQGEGDDLSDEEQELLAEADDARERSVVLFRDPSGFVNGGRWTAQRAFWGMVHNRIETLLAERFSVSGAAEKVRG

SEQ ID NO: 56 - protein, 1112 aa, Bacillus sp.
  source 1..1112: note = V3-13
MAIRSIKLKMKTNSGTDSIYLRKALWRTHQLINEGIAYYMNLLTLYRQEAIGDKTKEAYQAELINIIRNQQRNNGSSEEHGSDQEILALLRQLYELIIPSSIGESGDANQLGNKFLYPLVDPNSQSGKGTSNAGRKPRWKRLKEEGNPDWELEKKKDEERKAKDPTVKIFDNLNKYGLLPLFPLFTNIQKDIEWLPLGKRQSVRKWDKDMFIQAIERLLSWESWNRRVADEYKQLKEKTESYYKEHLTGGEEWIEKIRKFEKERNMELEKNAFAPNDGYFITSRQIRGWDRVYEKWSKLPESASPEELWKVVAEQQNKMSEGFGDPKVFSFLANRENRDIWRGHSERIYHIAAYNGLQKKLSRTKEQATFTLPDAIEHPLWIRYESPGGTNLNLFKLEEKQKKNYYVTLSKIIWPSEEKWIEKENIEIPLAPSIQFNRQIKLKQHVKGKQEISFSDYSSRISLDGVLGGSRIQFNRKYIKNHKELLGEGDIGPVFFNLVVDVAPLQETRNGRLQSPIGKALKVISSDFSKVIDYKPKELMDWMNTGSASNSFGVASLLEGMRVMSIDMGQRTSASVSIFEVVKELPKDQEQKLFYSINDTELFAIHKRSFLLNLPGEVVTKNNKQQRQERRKKRQFVRSQIRMLANVLRLETKKTPDERKKAIHKLMEIVQSYDSWTASQKEVWEKELNLLTNMAAFNDEIWKESLVELHHRIEPYVGQIVSKWRKGLSEGRKNLAGISMWNIDELEDTRRLLISWSKRSRTPGEANRIETDEPFGSSLLQHIQNVKDDRLKQMANLIIMTALGFKYDKEEKDRYKRWKETYPACQIILFENLNRYLFNLDRSRRENSRLMKWAHRSIPRTVSMQGEMFGLQVGDVRSEYSSRFHAKTGAPGIRCHALTEEDLKAGSNTLKRLIEDGFINESELAYLKKGDIIPSQGGELFVTLSKRYKKDSDNNELTVIHADINAAQNLQKRFWQQNSEVYRVPCQLARMGEDKLYIPKSQTETIKKYFGKGSFVKNNTEQEVYKWEKSEKMKIKTDTTFDLQDLDGFEDISKTIELAQEQQKKYLTMFRDPSGYFFNNETWRPQKEYWSIVNNIIKSCLKKKILSNKVEL

SEQ ID NO: 57 - protein, 1108 aa, Bacillus sp.
MAIRSIKLKLKTHTGPEAQNLRKGIWRTHRLLNEGVAYYMKMLLLFRQESTGERPKEELQEELICHIREQQQRNQADKNTQALPLDKALEALRQLYELLVPSSVGQSGDAQIISRKFLSPLVDPNSEGGKGTSKAGAKPTWQKKKEANDPTWEQDYEKWKKRREEDPTASVITTLEEYGIRPIFPLYTNTVTDIAWLPLQSNQFVRTWDRDMLQQAIERLLSWESWNKRVQEEYAKLKEKMAQLNEQLEGGQEWISLLEQYEENRERELRENMTAANDKYRITKRQMKGWNELYELWSTFPASASHEQYKEALKRVQQRLRGRFGDAHFFQYLMEEKNRLIWKGNPQRIHYFVARNELTKRLEEAKQSATMTLPNARKHPLWVRFDARGGNLQDYYLTAEADKPRSRRFVTFSQLIWPSESGWMEKKDVEVELALSRQFYQQVKLLKNDKGKQKIEFKDKGSGSTFNGHLGGAKLQLERGDLEKEEKNFEDGEIGSVYLNVVIDFEPLQEVKNGRVQAPYGQVLQLIRRPNEFPKVTTYKSEQLVEWIKASPQHSAGVESLASGFRVMSIDLGLRAAAATSIFSVEESSDKNAADFSYWIEGTPLVAVHQRSYMLRLPGEQVEKQVMEKRDERFQLHQRVKFQIRVLAQIMRMANKQYGDRWDELDSLKQAVEQKKSPLDQTDRTFWEGIVCDLTKVLPRNEADWEQAVVQIHRKAEEYVGKAVQAWRKRFAADERKGIAGLSMWNIEELEGLRKLLISWSRRTRNPQEVNRFERGHTSHQRLLTHIQNVKEDRLKQLSHAIVMTALGYVYDERKQEWCAEYPACQVILFENLSQYRSNLDRSTKENSTLMKWAHRSIPKYVHMQAEPYGIQIGDVRAEYSSRFYAKTGTPGIRCKKVRGQDLQGRRFENLQKRLVNEQFLTEEQVKQLRPGDIVPDDSGELFMTLTDGSGSKEVVFLQADINAAHNLQKRFWQRYNELFKVSCRVIVRDEEEYLVPKTKSVQAKLGKGLFVKKSDTAWKDVYVWDSQAKLKGKTTFTEESESPEQLEDFQEIIEEAEEAKGTYRTLFRDPSGVFFPESVWYPQKDFWGEVKRKLYGKLRERFLTKAR

SEQ ID NO: 58 - protein, 1090 aa, Laceyella sediminis
MSIRSFKLKIKTKSGVNAEELRRGLWRTHQLINDGIAYYMNWLVLLRQEDLFIRNEETNEIEKRSKEEIQGELLERVHKQQQRNQWSGEVDDQTLLQTLRHLYEEIVPSVIGKSGNASLKARFFLGPLVDPNNKTTKDVSKSGPTPKWKKMKDAGDPNWVQEYEKYMAERQTLVRLEEMGLIPLFPMYTDEVGDIHWLPQASGYTRTWDRDMFQQAIERLLSWESWNRRVRERRAQFEKKTHDFASRFSESDVQWMNKLREYEAQQEKSLEENAFAPNEPYALTKKALRGWERVYHSWMRLDSAASEEAYWQEVATCQTAMRGEFGDPAIYQFLAQKENHDIWRGYPERVIDFAELNHLQRELRRAKEDATFTLPDSVDHPLWVRYEAPGGTNIHGYDLVQDTKRNLTLILDKFILPDENGSWHEVKKVPFSLAKSKQFHRQVWLQEEQKQKKREVVFYDYSTNLPHLGTLAGAKLQWDRNFLNKRTQQQIEETGEIGKVFFNISVDVRPAVEVKNGRLQNGLGKALTVLTHPDGTKIVTGWKAEQLEKWVGESGRVSSLGLDSLSEGLRVMSIDLGQRTSATVSVFEITKEAPDNPYKFFYQLEGTELFAVHQRSFLLALPGENPPQKIKQMREIRWKERNRIKQQVDQLSAILRLHKKVNEDERIQAIDKLLQKVASWQLNEEIATAWNQALSQLYSKAKENDLQWNQAIKNAHHQLEPVVGKQISLWRKDLSTGRQGIAGLSLWSIEELEATKKLLTRWSKRSREPGVVKRIERFETFAKQIQHHINQVKENRLKQLANLIVMTALGYKYDQEQKKWIEVYPACQVVLFENLRSYRFSYERSRRENKKLMEWSHRSIPKLVQMQGELFGLQVADVYAAYSSRYHGRTGAPGIRCHALTEADLRNETNIIHELIEAGFIKEEHRPYLQQGDLVPWSGGELFATLQKPYDNPRILTLHADINAAQNIQKRFWHPSMWFRVNCESVMEGEIVTYVPKNKTVHKKQGKTFRFVKVEGSDVYEWAKWSKNRNKNTFSSITERKPPSSMILFRDPSGTFFKEQEWVEQKTFWGKVQSMIQAYMKKTIVQRMEE

SEQ ID NO: 59 - protein, 1108 aa, Bacillus hisashii
MATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 60 - protein, 1119 aa, unidentified
  source 1..1119: note = Spirochaetes bacterium
MSFTISYPFKLIIKNKDEAKALLDTHQYMNEGVKYYLEKLLMFRQEKIFIGEDETGKRIYIEETEYKKQIEEFYLIKKTELGRNLTLTLDEFKTLMRELYICLVSSSMENKKGFPNAQQASLNIFSPLFDAESKGYILKEENNNISLIHKDYGKILLKRLRDNNLIPIFTKFTDIKKITAKLSPTALDRMIFAQAIEKLLSYESWCKLMIKERFDKEVKIKELENKCENKQERDKIFEILEKYEEERQKTFEQDSGFAKKGKFYITGRMLKGFDEIKEKWLKEKDRSEQNLINILNKYQTDNSKLVGDRNLFEFIIKLENQCLWNGDIDYLKIKRDINKNQIWLDRPEMPRFTMPDFKKHPLWYRYEDPSNSNFRNYKIEVVKDENYITIPLITERNNEYFEENYTFNLAKLKKLSENITFIPKSKNKEFEFIDSNDEEEDKKDQKKSKQYIKYCDTAKNTSYGKSGGIRLYFNRNELENYKDGKKMDSYTVFTLSIRDYKSLFAKEKLQPQIFNTVDNKITSLKIQKKFGNEEQTNFLSYFTQNQITKKDWMDEKTFQNVKELNEGIRVLSVDLGQRFFAAVSCFEIMSEIDNNKLFFNLNDQNHKIIRINDKNYYAKHIYSKTIKLSGEDDDLYKERKINKNYKLSYQERKNKIGIFTRQINKLNQLLKIIRNDEIDKEKFKELIETTKRYVKNTYNDGIIDWNNVDNKILSYENKEDVINLHKELDKKLEIDFKEFIRECRKPIFRSGGLSMQRIDFLEKLNKLKRKWVARTQKSAESIVLTPKFGYKLKEHINELKDNRVKQGVNYILMTALGYIKDNEIKNDSKKKQKEDWVKKNRACQIILMEKLTEYTFAEDRPREENSKLRMWSHRQIFNFLQQKASLWGILVGDVFAPYTSKCLSDNNAPGIRCHQVTKKDLIDNSWFLKIVVKDDAFCDLIEINKENVKNKSIKINDILPLRGGELFASIKDGKLHIVQADINASRNIAKRFLSQINPFRVVLKKDKDETFHLKNEPNYLKNYYSILNFVPTNEELTFFKVEENKDIKPTKRIKMDKHEKESTDEGDDYSKNQIALFRDDSGIFFDKSLWVDGKIFWSVVKNKMTKLLRERNNKKNGSK

SEQ ID NO: 61 - protein, 9 aa, synthetic construct
PKKKRKVPG

SEQ ID NO: 62 - protein, 9 aa, synthetic construct
ASPKKKRKV

SEQ ID NO: 63 - DNA, 20 bp, Homo sapiens
  source 1..20: mol_type = unassigned DNA
ctcttctgggctccctacaa

SEQ ID NO: 64 - DNA, 20 bp, Homo sapiens
  source 1..20: mol_type = unassigned DNA
aaccactagcactagccttg

SEQ ID NO: 65 - DNA, 20 bp, Homo sapiens
  source 1..20: mol_type = unassigned DNA
tacaggaggcaataacagat

SEQ ID NO: 66 - DNA, 20 bp, Homo sapiens
  source 1..20: mol_type = unassigned DNA
aggccaaagaattcctggaa

SEQ ID NO: 67 - DNA, 20 bp, Homo sapiens
  source 1..20: mol_type = unassigned DNA
ggcacattaattcactgtgt

SEQ ID NO: 68 - DNA, 20 bp, Homo sapiens
  source 1..20: mol_type = unassigned DNA
caggtgacaggctaggcttc

SEQ ID NO: 69 - DNA, 20 bp, synthetic construct
  source 1..20: mol_type = other DNA
tcctgcactgaaactgttgc

SEQ ID NO: 70 - DNA, 20 bp, Homo sapiens
  source 1..20: mol_type = unassigned DNA
agatagtgtggggaaggggc

SEQ ID NO: 71 - DNA, 20 bp, Homo sapiens
  source 1..20: mol_type = unassigned DNA
gcattgagatagtgtgggga

SEQ ID NO: 72 - protein, 100 aa, Homo sapiens
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 73 - DNA, 20 bp, Mus musculus
  source 1..20: mol_type = unassigned DNA
gctgcccgccacactggcgc

SEQ ID NO: 74 - DNA, 20 bp, Mus musculus
  source 1..20: mol_type = unassigned DNA
ggcgtggtgatgctagggat

SEQ ID NO: 75 - DNA, 20 bp, Mus musculus
  source 1..20: mol_type = unassigned DNA
tagtctgctcaggatgaagc

SEQ ID NO: 76 - DNA, 20 bp, Mus musculus
  source 1..20: mol_type = unassigned DNA
aatcctgcccactgtgcagg

SEQ ID NO: 77 - DNA, 20 bp, Mus musculus
  source 1..20: mol_type = unassigned DNA
ccttggatcagaatccgcag

SEQ ID NO: 78 - DNA, 19 bp, synthetic construct
  source 1..19: mol_type = other DNA
tcctgagaagcgacctgga

SEQ ID NO: 79 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQFIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGRFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVALGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIRQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 80 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQFIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGRFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVALGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIRQIEYLERQYKFLKSWSFFGKVSGQVIRAEKGSRFAITLREHIDHAKEDRLKKLADRIIMEALGYVYALDDERGKGKWVAKYPPCQLILLAELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 81 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQFIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGRFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVALGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIRQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHAALNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 82 - protein, 7 aa, synthetic construct
PKKKRKV